(19) United States
(12) Patent Application Publication
(10) Pub. No.: US 2025/0303172 A1
(43) Pub. Date: Oct. 2, 2025

(54) VAGUS NERVE STIMULATION SYSTEMS AND METHODS

(71) Applicant: THE ALFRED E. MANN FOUNDATION FOR SCIENTIFIC RESEARCH, VALENCIA, CA (US)

(72) Inventors: Brian M. Shelton, Ventura, CA (US); Hitesh Hotlani, Santa Clarita, CA (US); Jason Goldberg, Oakland, CA (US); Leslie Farkas, Ventura, CA (US); Neil Talbot, La Crescenta, CA (US); Kenneth Shane Guillory, Salt Lake City, UT (US); Brian R. Dearden, Pasadena, CA (US); Susanna M. Lin, Los Angeles, CA (US); Joseph L. Calderon, Santa Clarita, CA (US); Edward Hillery, Valencia, CA (US); Jeffrey Brydle, Pasadena, CA (US); Anthony Ng, Santa Clarita, CA (US)

(21) Appl. No.: 19/090,259

(22) Filed: Mar. 25, 2025

Related U.S. Application Data
(60) Provisional application No. 63/569,878, filed on Mar. 26, 2024.

(51) Int. Cl. A61N 1/36; A61N 1/05; A61N 1/378

(52) U.S. Cl. CPC A61N 1/36139; A61N 1/0556; A61N 1/36053; A61N 1/36064; A61N 1/36096; A61N 1/3787

(57) ABSTRACT

A stimulation system includes an implantable medical device (IMD) configured to provide electrical stimulation to tissue; a controller configured to control the electrical stimulation provided by the IMD; an implantable sensor configured to measure first heart rate data and to transmit the first heart rate data to the controller; an external sensor configured to measure second heart rate data and to transmit the second heart rate data to the controller; and an external electronic device communicatively coupled to the IMD, wherein the controller is configured to selectively operate the IMD in an internal sensor mode, whereby the controller controls the electrical stimulation provided by the IMD based on the first heart rate data, or an external sensor mode, whereby the controller controls the electrical stimulation provided by the IMD based on the second heart rate data.

### CROSS-REFERENCE TO RELATED APPLICATION

This application claims priority to, and the benefit of, U.S. Provisional Patent Application No. 63/569,878, filed on Mar. 26, 2024, the entire content of which is hereby incorporated by reference.

### BACKGROUND

Epilepsy and depression are two common maladies. Epilepsy produces potentially-fatal seizures. Both conditions can be treated under appropriate circumstances with vagus nerve stimulation. Vagus nerve stimulation may entail the surgical implantation of a stimulator device into a patient's chest area under the skin to stimulate the vagus nerve with electrical stimulation pulses. The vagus nerve originates from the brainstem and traverses both sides of the neck down to the chest and abdomen. The stimulator device sends electrical signals via the vagus nerve to the brain. A stimulation lead having a nerve cuff at the proximal end thereof connects the stimulator device to the vagus nerve. The nerve cuff has one or more electrodes within it and, when implanted, at least partly encircles the vagus nerve. Vagus nerve stimulation has been shown to be helpful in many cases for reducing the number and severity of seizures, particularly for patients who are less responsive to more non-invasive methods like oral medication. Vagus nerve stimulation has also been shown to reduce depression in certain treatment-resistant patients.

There is an ongoing need to improve systems for providing vagus nerve stimulation, and it is in view of this technical background that the present disclosure is provided. This background section is provided solely to introduce certain background material relating to the present disclosure and, thus, is not an admission of prior art.

### SUMMARY

This summary section introduces some features of nonlimiting and non-exhaustive examples of the present disclosure, and is not intended to limit the scope of the claims.

According to an aspect, the present disclosure relates to a stimulation system including an implantable medical device (IMD) configured to provide electrical stimulation to tissue; a controller configured to control the electrical stimulation provided by the IMD; an implantable sensor configured to measure first heart rate data and to transmit the first heart rate data to the controller; an external sensor configured to measure second heart rate data and to transmit the second heart rate data to the controller; and an external electronic device communicatively coupled to the IMD, wherein the controller is configured to selectively operate the IMD in an internal sensor mode, whereby the controller controls the electrical stimulation provided by the IMD based on the first heart rate data, or an external sensor mode, whereby the controller controls the electrical stimulation provided by the IMD based on the second heart rate data.

In some examples, the controller is configured to not operate, or to not communicate with, the implantable sensor when operating the IMD in the external sensor mode.

In some examples, the controller is configured to operate the IMD in the internal sensor mode in response to determining that a threshold criterion is satisfied.

In some examples, the threshold criterion includes the controller being communicatively disconnected from the external sensor for a threshold time period and/or a distance between the external sensor and an implantable component of the stimulation system being greater than a threshold distance.

In some examples, the threshold criterion includes at least one of the following: a signal quality of the second heart rate data being below a threshold value; a scheduled day and/or intraday time period; the implantable sensor being ranked higher than the external sensor; a battery level of the external sensor being below a threshold level; or a processing capability value of the external sensor being below a threshold value.

In some examples, the electronic device is configured to receive user input via a user interface, and the threshold criterion includes receiving a user control signal from the electronic device.

In some examples, the controller is configured to operate the IMD in the external sensor mode in response to determining that a threshold criterion is satisfied.

In some examples, the threshold criterion includes the controller being communicatively connected to the external sensor for a threshold time period and/or a distance between the external sensor and an implantable component of the stimulation system being less than a threshold distance.

In some examples, the threshold criterion includes at least one of the following: a signal quality of the second heart rate data being above a threshold value; a scheduled day and/or intraday time period; the external sensor being ranked higher than the implantable sensor; a battery level of the external sensor being above a threshold level; or a processing capability value of the external sensor being above a threshold value.

In some examples, the controller is further configured to selectively operate the IMD in a multi-sensor mode, whereby the controller controls the electrical stimulation provided by the IMD based on both the first heart rate data and the second heart rate data.

In some examples, the controller is part of the IMD.

In some examples, the controller is part of the electronic device.

In some examples, the external sensor is part of the electronic device.

In some examples, the external sensor is separate from the electronic device.

In some examples, the implantable sensor is part of the IMD.

In some examples, the implantable sensor is separate from the IMD.

In some examples, the implantable sensor includes at least one of an inertial measurement unit (IMU) or an accelerometer.

In some examples, the electronic device includes a wearable device selected from among a watch, a ring, a bracelet, a band, a necklace, or an earring.

In some examples, the electronic device includes a stationary device, configured to be operated while positioned on a surface, or a portable device, configured to be operated while being held or carried.

In some examples, the IMD includes a cuff electrode configured to stimulate a vagus nerve, the cuff electrode including a plurality of electrode contacts configured to circumferentially surround the vagus nerve.

In some examples, the controller is configured to independently activate each of the plurality of electrode contacts as a cathode or as an anode.

In some examples, the IMD includes a conductive housing containing at least some components of the IMD and being exposed to an outside of the IMD, and the controller is configured to selectively activate the conductive housing as an anode.

In some examples, the IMD includes a receiver coil, and wherein the stimulation system includes a wireless power transfer device, including a first coil oriented along a first axis; a second coil oriented along a second axis different from the first axis and positioned above the first coil along a direction perpendicular to the first and second axes; and a driver configured to differentially drive the first and second coils to generate a magnetic field and to control a direction of the magnetic field at the receiver coil.

In some examples, the wireless power transfer device includes a transmission component housing the first and second coils; an electronics component housing the driver; and a cable physically and electrically connecting the electronics component to the transmission component.

In some examples, the system includes a support garment for the wireless power transfer device, the support garment including a first chest part configured to cover a first sagittal side of a wearer's chest; a second chest part configured to cover a second sagittal side of the wearer's chest; and a neck part coupled between the first and second chest parts and configured to cover a back of the wearer's neck.

In some examples, the support garment includes at least one of a first fastener on the first chest part and configured to attach to the transmission component; a second fastener on the second chest part and configured to attach to the electronics component; or a cable holder on the neck part and configured to secure the cable along the neck part.

According to an aspect, the present disclosure relates to a stimulation system, including an implantable medical device (IMD) comprising: an implantable pulse generator (IPG) configured to generate a stimulation current, a stimulation lead coupled to the IPG, and a stimulation electrode on the stimulation lead and configured to receive the stimulation current from the IPG through the stimulation lead; a controller; an implantable sensor configured to measure first biometric data and to transmit the first biometric data to the controller; an external sensor configured to measure second biometric data and to transmit the second biometric data to the controller; and an external electronic device communicatively coupled to the implantable stimulator and configured to receive input data via a user interface, wherein the controller is configured to control the stimulation current generated by the IPG based selectively on the first biometric data or the second biometric data.

In some examples, the controller is further configured to selectively control the stimulation current generated by the IPG based on the first biometric data, based on the second biometric data, or based on both the first and second biometric data.

In some examples, the controller is configured to determine a heart rate based on at least one of the first biometric data or the second biometric data.

In some examples, the controller is configured to perform a titration process. Titration may refer to gradually adjusting one or more parameters such as the amplitude, frequency, or duration of electrical stimulation, to determine the optimal settings for achieving the desired therapeutic effect. In some examples, the titration process includes determining a normal heart rate value based on at least one of the first biometric data or the second biometric data; performing an iterative neural fulcrum identification (NFI) operation, including (a) generating the stimulation current having a set amplitude, (b) determining a transient heart rate value based on at least one of the first biometric data or the second biometric data measured while providing the stimulation current of process (a), and (c) determining a heart rate change (HRC) value based on the normal heart rate value and the transient heart rate value of process (b); performing the NFI operation one or more additional times, each time at a higher set amplitude than the previous time; and determining, based on the plurality of HRC values determined during the NFI operations, a neural fulcrum amplitude associated with a neural fulcrum response.

In some examples, the controller is configured to generate the pulse stimulation current with an amplitude based on the neural fulcrum amplitude.

In some examples, the NFI operation includes a process (n), before process (a), of determining a normal heart rate value while the stimulation current is not provided or is provided with an amplitude less than the set amplitude of process (a), and the heart rate change value of process (c) is determined using the normal heart rate value determined during process (n).

In some examples, an inter-NFI time period between processes (a) of two adjacently performed NFI operations is less than 4 hours.

In some examples, the inter-NFI time period is less than 30 minutes.

In some examples, the NFI operation includes a process (d) of detecting for an electromyography (EMG) response while the stimulation current is provided during process (a), the titration process includes determining a lowest EMG amplitude that triggers an EMG response, and the controller is configured to generate the stimulation current with an amplitude based on both the neural fulcrum amplitude and the lowest EMG amplitude.

In some examples, the controller is configured to detect a seizure, or the onset of a seizure, based on a comparison of the determined heart rate and a personalized ictal tachycardia model.

In some examples, the system includes a memory coupled to the controller and storing the personalized ictal tachycardia model.

In some examples, the personalized ictal tachycardia model is based on a plurality of sets of seizure data, each of the sets of seizure data including heart rate data of a single subject while having a seizure.

In some examples, the controller is configured to record, for each of a plurality of seizures, a corresponding set of seizure data including heart rate data determined based on at least one of the first biometric data or the second biometric data; and to generate the personalized ictal tachycardia model based on the plurality of sets of seizure data.

In some examples, the controller is configured to determine a respiration rate based on at least one of the first biometric data or the second biometric data, and to detect the seizure, or the onset of the seizure, based further on the determined respiration rate and a personalized ictal apnea respiration response model.

In some examples, the personalized ictal tachycardia model includes a model parameter, and the detecting the seizure, or the onset of the seizure, includes calculating a heart rate parameter, based on at least one of the first biometric data or the second biometric data, and comparing the heart rate parameter to the model parameter.

In some examples, the ictal tachycardia model includes a discriminative neural network configured to detect the seizure, or the onset of the seizure, based on heart rate data determined based on at least one of the first biometric data or the second biometric data.

In some examples, the implantable sensor or the external sensor includes at least one of an inertial measurement unit (IMU) or an accelerometer configured to measure movement data, the controller is configured to detect a fall event based on the movement data, and to detect ictal tachycardia based on at least one of the first biometric data or the second biometric data.

In some examples, the controller is configured to detect the ictal tachycardia based on the movement data.

In some examples, the controller is configured to cause the IMD to begin generating the stimulation current, or to increase a parameter of the stimulation current, in response to detecting both the fall event and the ictal tachycardia.

In some examples, the controller is configured to not cause the IMD to begin generating the stimulation current, or to not increase the parameter of the stimulation current, in response to detecting the fall event without detecting the ictal tachycardia.

In some examples, the system includes a transmitter, and the controller is configured to transmit, via the transmitter, an alert signal in response to detecting the fall event.

In some examples, the system is configured to recalibrate the implantable sensor based on the second biometric data and/or to recalibrate the external sensor based on the first biometric data.

In some examples, the system is configured to compare the first biometric data to the second biometric data to determine that one of the implantable sensor or the external sensor is uncalibrated.

In some examples, the controller is an implantable controller that is part of the implantable stimulator, the electronic device includes an external controller configured to receive the first and second biometric data, and the stimulation system is configured to selectively operate in a first mode, whereby the implantable controller determines, based on at least one of the first biometric data or the second biometric data, a stimulation parameter for the stimulation current, or in a second mode, whereby the external controller determines, based on at least one of the first biometric data or the second biometric data, the stimulation parameter for the stimulation current.

In some examples, the stimulation system is configured to transition from the first mode to the second mode in response to a communication link between the implantable controller and the external controller being established, and to transition from the second mode to the first mode in response to implantable controller being communicatively disconnected from the external controller for a threshold time period.

According to an aspect, the technology relates to a method for providing electrical stimulation to tissue of a subject via an implantable medical device (IMD) implanted within the subject, the method including controlling, via a controller and during a first time period, a parameter of the electrical stimulation based on first heart rate data measured by an implantable sensor implanted within the subject; and controlling, via the controller and during a second time period, the parameter of the electrical stimulation based on second heart rate data measured by an external sensor external to the subject.

In some examples, during the second time period, the implantable sensor is not operated or is not communicatively coupled to the controller.

In some examples, during the first time period, the external sensor is communicatively disconnected from the controller.

In some examples, the method includes controlling, via the controller and during a third time period, the parameter of the electrical stimulation based on both the first heart rate data and the second heart rate data.

### BRIEF DESCRIPTION OF THE DRAWINGS

The drawings, together with the specification, illustrate nonlimiting and non-exhaustive examples of the present disclosure.

FIG. 1 is a schematic view of an implantable medical device (IMD) system according to some examples.

FIG. 2A is a diagram illustrating a vagus nerve stimulator (VNS) according to some examples and when subcutaneously implanted in the chest of a patient.

FIG. 2B is a schematic diagram of an implantable pulse generator (IPG) of the VNS of FIG. 2A according to some examples.

FIG. 3A depicts a VNS, according to some examples, and schematically depicts certain external electronic devices of a VNS system according to some examples.

FIG. 3B depicts a stimulation lead and nerve cuff of the VNS of FIG. 3A according to some examples.

FIG. 3C is an end view of the nerve cuff of the VNS of FIG. 3B when the nerve cuff is in a furled state, according to some examples.

FIG. 3D is a schematic view of the IPG of the VNS of FIG. 3A according to some examples.

FIG. 4A is a schematic view of some components of an external charger and of an IPG, according to some examples.

FIG. 4B is a perspective view of first and second transmitting coils of the external charger of FIG. 4A according to some examples.

FIG. 4C is a top-down view of the first and second transmitting coils of FIG. 4B.

FIG. 4D is more detailed schematic view of some components of the external charger and IPG of FIG. 4A according to some examples.

FIG. 4E is a front view of the external charger of FIG. 4D according to some examples.

FIG. 4F is a rear view of the external charger of FIG. 4D according to some examples.

FIG. 5A depicts a charger support garment, according to some examples and being worn on a patient, and the external charger attached to the charger support garment.

FIG. 5B is a schematic view of a VNS system according to some examples.

FIG. 6A is a schematic view of a VNS system according to some examples.

FIG. 6B is a schematic view of a first example of the VNS system of FIG. 6A.

FIG. 6C is a schematic view of a second example of the VNS system of FIG. 6A.

FIG. 7 is a flowchart of a method relating to a processing workflow for determining whether an external sensor should be selected to replace an implantable sensor according to some examples.

FIG. 8 is a flowchart diagram for a method of selectively using an external sensor or an implantable sensor for controlling electrical stimulation by an IMD according to some examples.

FIG. 9 is a flowchart of a method for recalibrating a sensor according to some examples.

FIG. 10 is a flowchart of another method for recalibrating a sensor according to some examples.

FIG. 11 is a flowchart diagram of a method for a VNS system selectively operating in an internal controller mode or an external controller mode, according to some examples.

FIG. 12 is a flowchart diagram of a method for providing electrical stimulation according to some examples.

FIG. 13A is a schematic view of a VNS according to some examples.

FIG. 13B is a schematic view of some components of the VNS of FIG. 13A.

FIG. 14A shows a graph of a parametrized model of ictal tachycardia according to some examples.

FIG. 14B shows a graph of an exponential model of respiration rate according to some examples.

FIG. 15 is a schematic view of a system or method for detecting the occurrence or onset of a seizure and for performing closed-loop vagus nerve stimulation according to some examples.

FIG. 16 is a schematic view of a machine learning model according to some examples.

FIG. 17 shows a graph of heart rate change (vertical axis) versus pulse amplitude (horizontal axis) of pulse stimulation according to some examples.

FIG. 18 is a flowchart showing a method for VNS titration according to some examples.

FIG. 19 is a flowchart diagram of a method for providing stimulation treatment according to some examples.

FIG. 20 is a flowchart diagram of a method for detecting a fall event, and for using the detected fall event, in the operations of a VNS system according to some examples.

FIG. 21 is a block diagram of various components of a computer system according to some examples.

### DETAILED DESCRIPTION

The present disclosure relates to systems and methods for providing vagus nerve stimulation that provide various improvements over conventional systems and methods.

Various details of additional components, methods, and configurations that may be used in conjunction with the example vagus nerve stimulator (VNS) systems and methods of providing vagus nerve stimulation described herein are disclosed in U.S. Pat. No. 7,519,419, entitled MATERIAL AND METHOD OF FORMING YTTRIA-STABILIZED ZIRCONIA TO MINIMIZE LOW-TEMPERATURE DEGRADATION and issued Apr. 14, 2009; U.S. Pat. No. 8,140,162, entitled LOW-TEMPERATURE DEGRADATION RESISTANT YTTRIA STABILIZED ZIRCONIA and issued Mar. 20, 2012; U.S. Pat. No. 6,997,071, entitled NON-DESTRUCTIVE METHOD OF PREDICTING PERFORMANCE OF CERAMIC COMPONENTS and issued Feb. 14, 2006; U.S. Pat. No. 11,990,772, entitled SELF ALIGNING MAGNETIC FIELD SYSTEM FOR WIRELESS POWER TRANSFER and issued on May 21, 2024; U.S. Utility Patent Application Publication No. 2023/0248302, entitled SYSTEMS AND METHODS FOR VAGUS NERVE MONITORING AND STIMULATION and published Aug. 10, 2023; U.S. Patent Application Publication No. 2023/0299620, entitled AUTOMATICALLY-ALIGNING MAGNETIC FIELD SYSTEM and published Sep. 21, 2023; U.S. Patent Application Publication No. 2024/0079147, entitled SYSTEMS AND METHODS OF DEDUPLICATING DATA COLLECTED BY AN IMPLANTABLE MEDICAL DEVICE and published Mar. 7, 2024; U.S. Patent Application Publication No. 2024/0189610, entitled SYSTEM AND METHOD FOR DETERMINING AND ALERTING A PATIENT TO A LOW BATTERY CONDITION IN AN IMPLANTABLE PULSE GENERATOR (IPG) and published on Jun. 13, 2024; U.S. Patent Application Publication No. 2024/0113534, entitled WAKE-ABLE ELECTRONIC DEVICE AND METHODS FOR WAKING THEREOF and published on Apr. 4, 2024; U.S. Patent Application Publication No. 2024/0033524, entitled IMPLANTABLE MEDICAL DEVICE HAVING AN INERTIAL SENSING UNIT and published on Feb. 1, 2024; U.S. patent application Ser. No. 18/601,594, entitled IMPLANTABLE MEDICAL DEVICES AND HEADER ASSEMBLIES FOR USE WITH SAME and filed on Mar. 11, 2024; U.S. Patent Application Publication No. 2024/0316349, entitled SYSTEMS AND METHODS FOR MANAGING WIRELESS COMMUNICATION WITH IMPLANTED MEDICAL DEVICES and published on Sep. 26, 2024; US Patent Application Publication No. 2025/0025702, entitled IMPLANTABLE MEDICAL DEVICE CHARGER APPARATUS WITH WEARABLE CHARGER SUPPORTS and published on Jan. 23, 2025; U.S. Provisional Patent Application No. 63/548,673, entitled IMPLANTABLE STIMULATION SYSTEM WITH HEART RATE DETECTION and filed Feb. 1, 2024; and U.S. Provisional Patent Application Ser. No. 63/548,739, entitled SYSTEMS AND METHODS FOR SEIZURE DETECTION IN CLOSED-LOOP VAGUS NERVE STIMULATION and filed Feb. 1, 2024. The entire content of each of these patents, publications, and applications is hereby incorporated by reference.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several examples according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software may depend upon the particular application and design constraints imposed on the overall system.

By way of example, an element, or any portion of an element, or any combination of elements may be implemented as a “processing system” or “controller”. Any of the controllers described herein may include one or more processors. Examples of such processors include general purpose processors, microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, any combination of such components, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in some examples, the functions described herein may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, such computer-readable media (e.g., a memory) can include volatile memory, non-volatile memory, a random-access memory (RAM), a dynamic random access memory (DRAM), a static random access memory (SRAM), read only memory, flash or other solid state memory, a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

It will be understood that, although the terms “first”, “second”, “third”, etc., may be used herein to describe various elements, processes, or other features, these elements, processes, or features should not be limited by these terms. These terms are only used to distinguish one element, process, or feature from another element, process, or feature. Thus, a first element, process, or feature discussed herein could be termed a second element, process, or feature, without departing from the spirit and scope of the present disclosure.

Unless otherwise defined, all terms (including technical and scientific terms) used herein have the same meaning as commonly understood by one of the ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or in operation, in addition to the orientation depicted in the figures.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” specify the presence of stated elements, processes, and/or other features, but do not preclude the presence or addition of one or more other elements, processes, and/or features. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element is referred to as being “on”, “connected to”, “coupled to”, “attached to”, or “adjacent to” another element, it can be directly on, connected to, coupled to, attached to, or adjacent to the other element, or one or more intervening element(s) may be present. In contrast, when an element is referred to as being “directly on,” “directly connected to”, “directly coupled to”, “directly attached to”, or “immediately adjacent to” another element, there are no intervening elements present. Similar terms and phrases should be understood in a similar manner to encompass both direct and indirect affiliations between two or more elements being discussed. In addition, it will also be understood that when an element is referred to as being “between” two elements, it can be the only element between the elements, or one or more intervening elements may also be present.

As used herein, the phrase “at least part” includes part or all of the stated item, the phrase “at least partly” includes the stated item partly or entirely, and similar phrases should be interpreted in a similar manner.

As used herein, the term “substantially” and similar terms are used as terms of approximation and not as terms of degree, and are intended to account for the inherent deviations in measured or calculated values that would be recognized by those of ordinary skill in the art. Also, the term “about” and similar terms, when used herein in connection with a numerical value or a numerical range, are inclusive of the stated value and mean within an acceptable range of deviation for the particular value as determined by one of ordinary skill in the art, considering the measurement in question and the error associated with measurement of the particular quantity (e.g., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Controllers and similar components described herein may include any combination of hardware, firmware, and software, employed to process data or digital signals. Processing unit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs).

The various devices and components of the VNS systems described herein may be implemented utilizing any suitable hardware, firmware (e.g. an application-specific integrated circuit), software, or a combination of software, firmware, and hardware. For example, the various components of the VNS systems may be formed on one integrated circuit (IC) chip or on separate IC chips. Further, the various components of the VNS systems may be implemented on a flexible printed circuit film, a tape carrier package (TCP), a printed circuit board (PCB), or formed on one substrate. Further, the various components of the VNS systems may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a RAM. The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

### I. VNS System Overview

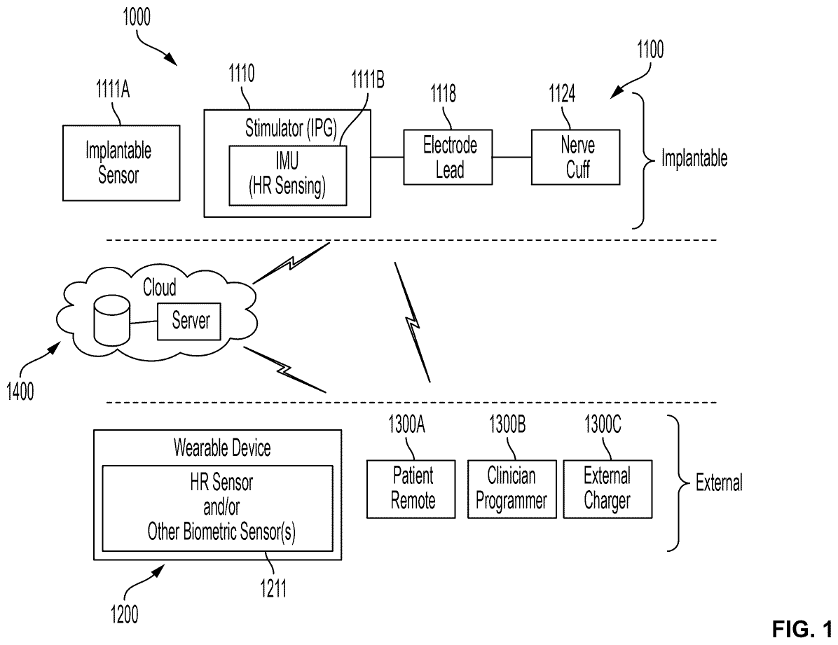

FIG. 1 is a schematic view of a medical system 1000 according to some examples. The medical system 1000 includes an implantable medical device (IMD) 1100, a first implantable sensor 1111A, an external wearable device 1200, various external electronic devices 1300A, 1300B, and 1300C, and a server 1400. Medical systems within the scope of the present disclosure can include any combination of these components and of other components described herein. The IMD 1100 will be primarily described herein as a vagus nerve stimulator (VNS) that is configured to provide electrical stimulation to the vagus nerve, and the medical system 1000 will be primarily described herein as a VNS system to treat epilepsy and seizures associated with epilepsy. However, the IMD 1100 may be any other type of IMD, such as any IMD configured to provide stimulation to biological tissue (e.g., nerve tissue, muscle tissue, organ tissue, etc.).

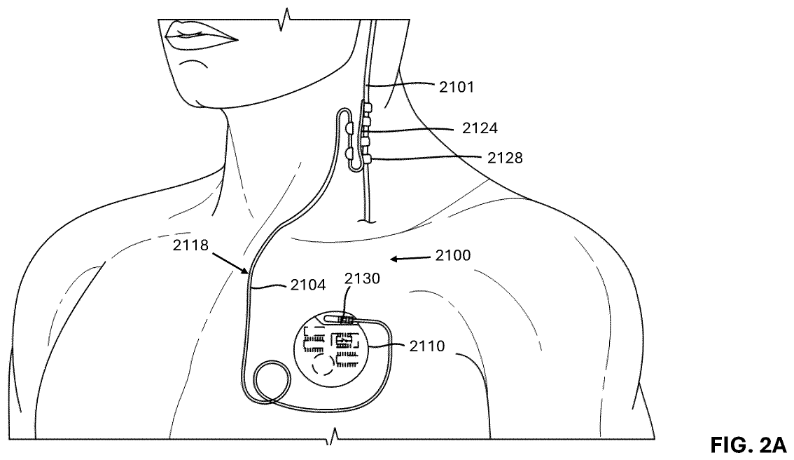

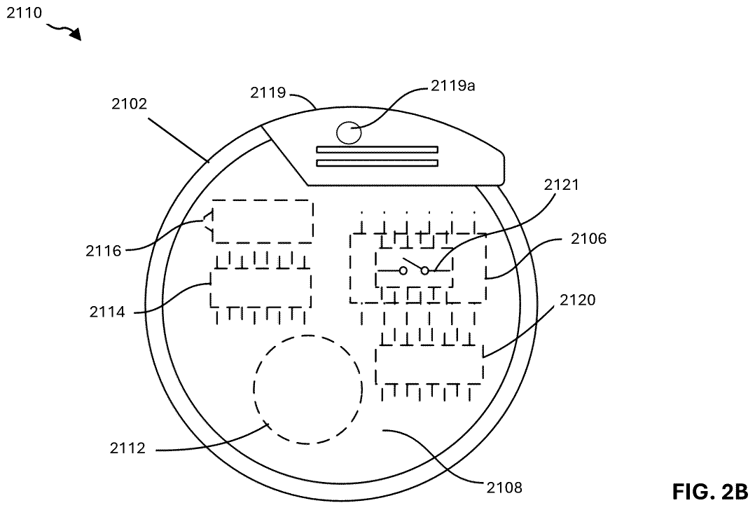

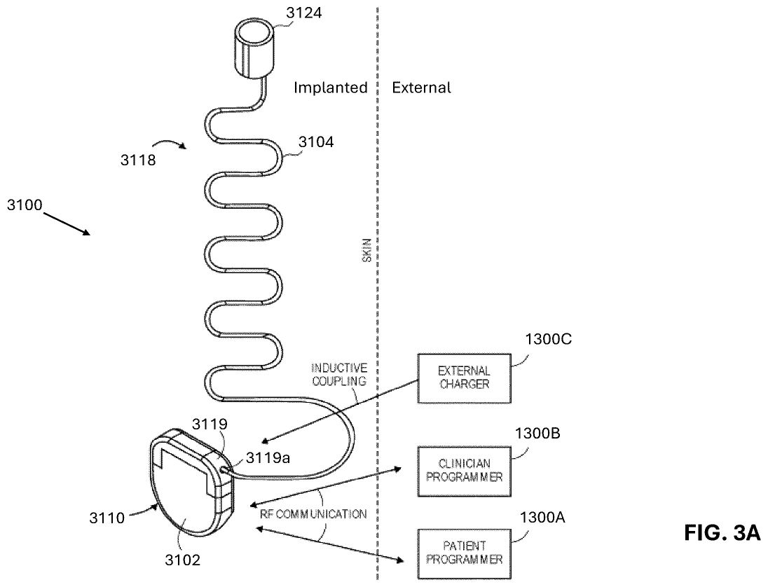

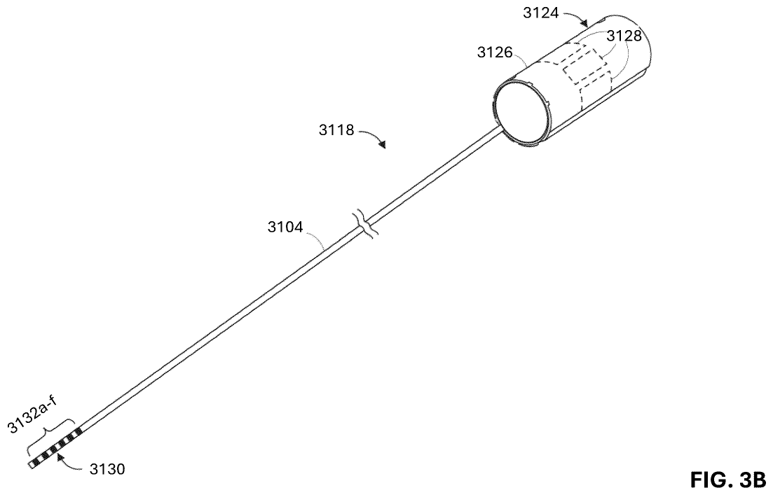

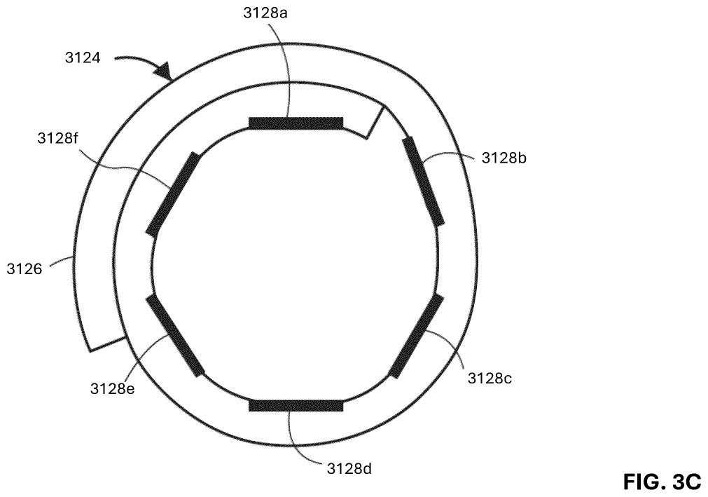

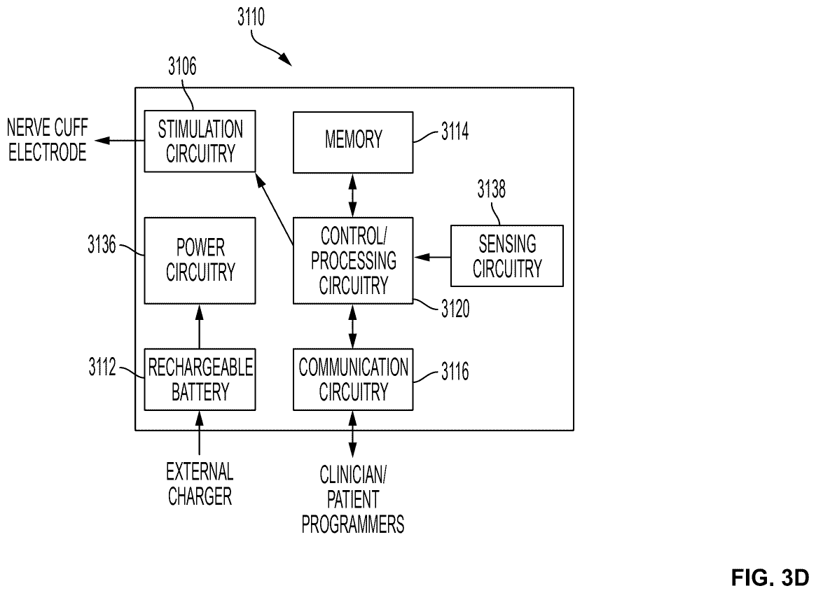

The VNS 1100 may be configured to be implanted in a patient and may include a stimulator 1110 (also referred to herein as an implantable pulse generator (IPG)) electrically coupled, via an electrode lead 1118, to a stimulation electrode 1124 (a nerve cuff in this example). The stimulator 1110 and the stimulation electrode 1124 will be primarily described herein as an implantable pulse generator (IPG) and a nerve cuff, respectively. However, the stimulator 1110 may include any type of device configured to generate electrical stimulation, and the nerve cuff 1124 may include any type of electrode configured to provide the generated electrical stimulation to any particular biological tissue. The VNS 1100 may be configured to wirelessly communicate with (e.g., wirelessly transmit data to) the wearable device 1200, any of the electronic devices 1300A, 1300B, and 1300C, and/or to the server 1400. For example, the VNS 1100 may be configured to communicate directly with the server 1400 (e.g., via a wifi or radio (including Bluetooth) connection) or indirectly with the server 1400 via one or more of the external devices 1200, 1300A, 1300B, and 1300C (e.g., as a conduit). Examples of the VNS will be described in more detail below with reference to FIGS. 2A-3D.

In some examples, the VNS 1100 may include a second implantable sensor 1111B integrally incorporated into the VNS 1100, for example, with the IPG 1110. The first implantable sensor 1111A may be separate from the VNS 1100 and may be configured to wirelessly communicate (e.g., via a wireless transmitter or transceiver, such as a Bluetooth radio or via RF signals) with other components of the VNS system 1000. For example, the first implantable sensor 1111A may be positioned in a patient's body spaced apart from the VNS 1100 and may be configured to wirelessly transmit sensed data to the VNS 1100, to the wearable device 1200, to any one or more of the electronic devices 1300A, 1300B, and 1300C, and/or to the server 1400. Each of the first and second implantable sensors 1111A and 1111B may independently be configured to sense (e.g., measure) biometric data (e.g., physiological parameters and biomarkers), which may include at least one of heart rate, heart rate variability, respiration (e.g., respiration rate), patient movement (e.g., patient activity and/or mobility), patient position, blood pressure, or oxygen saturation (SpO2). In some examples, each of the first and second implantable sensors 1111A and 1111B may include at least one of an inertial measurement unit (IMU), an accelerometer, or a gyroscope.

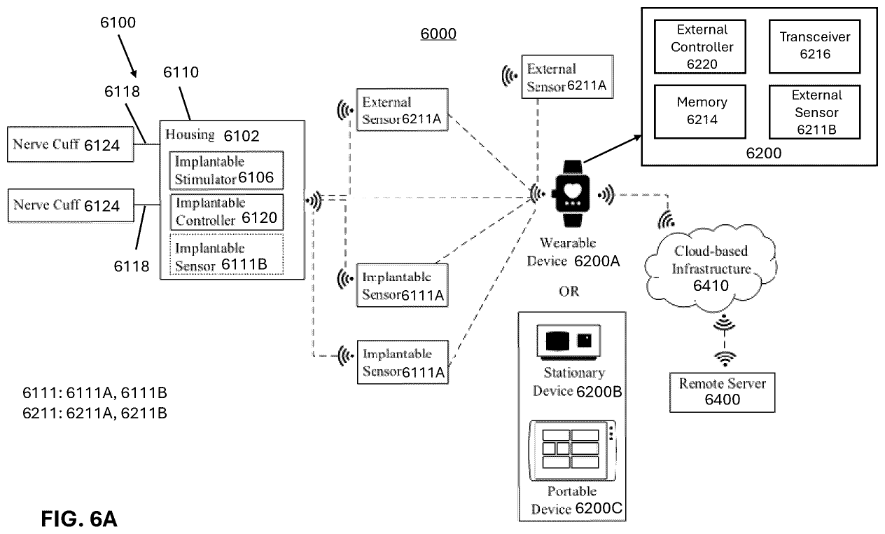

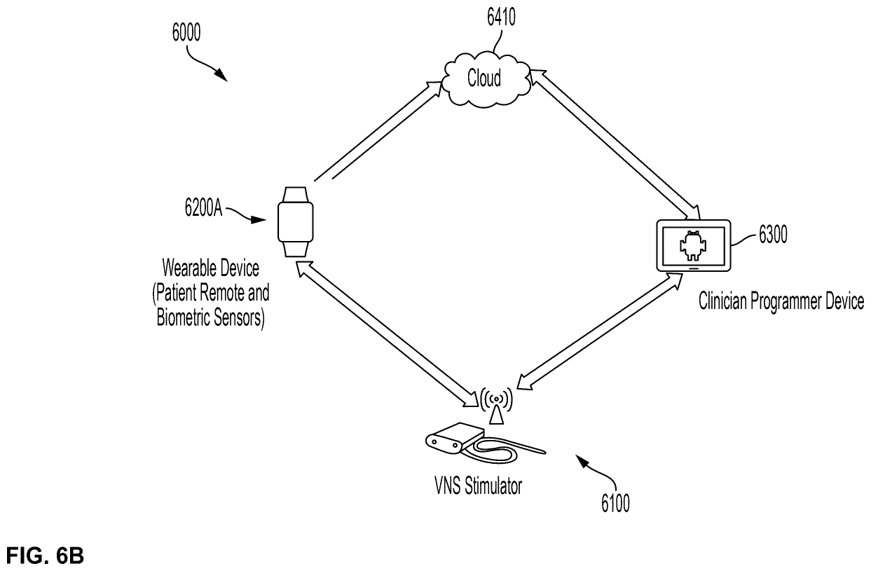

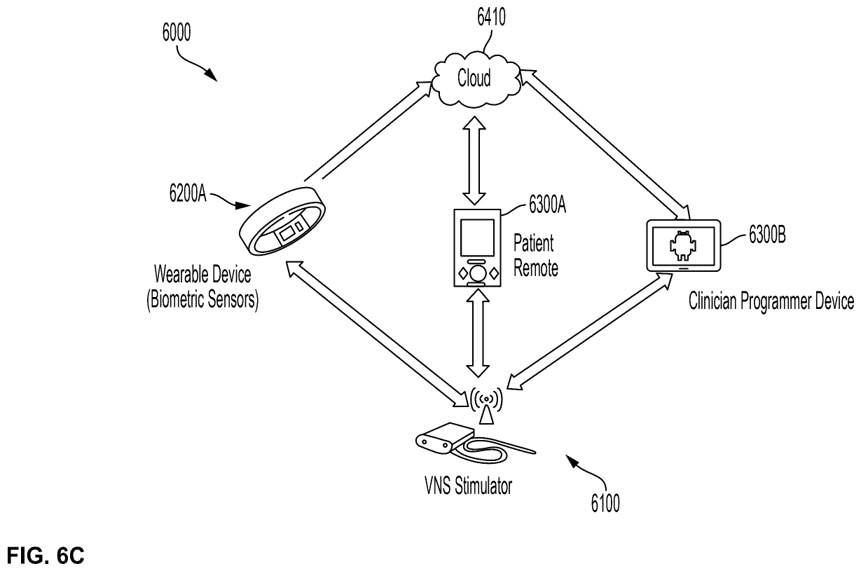

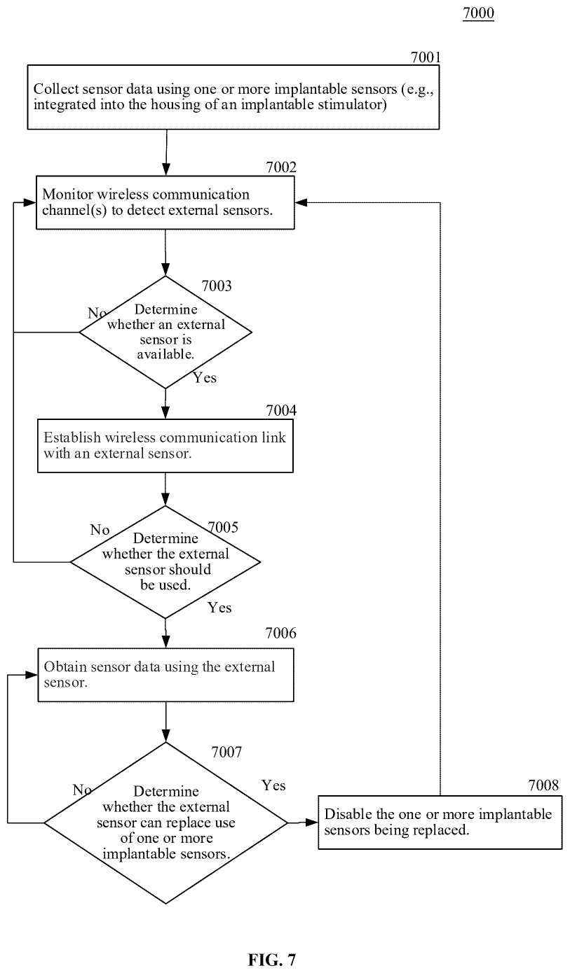

The wearable device 1200 may include any type of wearable device, such as a watch, a ring, a bracelet, a band, a necklace, or an earring. In some examples, the wearable device 1200 includes smart technology (e.g., a smart watch). The wearable device 1200 may include one or more external sensors 1211 configured to sense (e.g., measure) various types of biometric data, including those sensed by the first and second implantable sensors 1111A and 1111B, as well as additional biometric data. In some examples, the external sensor(s) 1211 may include at least one of an IMU, an accelerometer, or a gyroscope. The wearable device 1200 may be configured to wirelessly communicate (e.g., via a wireless transmitter or transceiver, such as a Bluetooth radio or via RF signals) with other components of the VNS system 1000, such as the VNS 1100, the first implantable sensor 1111A, any one or more of the external electronic devices 1300A, 1300B, or 1300C, and/or the server 1400. For example, the wearable device 1200 may be configured to transmit data sensed via the external sensor 1211 to the server 1400 or to the VNS 1100. Additional details, features, and examples of wearable devices, implantable sensors, and external sensors will be discussed herein below with reference to FIGS. 6A-6C.

The external electronic devices may include a patient remote 1300A (also referred to herein as a patient programmer), a clinician programmer 1300B, and an external charger 1300C. The patient remote 1300A may include a user interface (e.g., a speaker, a display such as a touch screen, one or more user input devices, etc.) configured to allow the patient to access, view, and/or control one or more settings or functions of the VNS 1100. The patient remote 1300A may comprise a dedicated hardware device, or software executed on a general-purpose computer or other electronic device (e.g., software executed on a tablet or laptop computer) capable of wirelessly communicating with the VNS 1100. In the depicted example, the wearable device 1200 and the patient remote 1300A are separate devices. However, in some other examples, the wearable device 1200 and the patient remote 1300A are integrated into a single device. For example, the wearable device 1200 may be configured to provide dual functionalities of (a) measuring biometric data (e.g., sensing heart rate, heart rate variability, respiration, SpO2, blood pressure, etc.), and (b) controlling the VNS 1100. This can eliminate the need for a separate patient remote.

The clinician programmer 1300B may have features similar to, or the same as, features of the patient remote 1300A. For example, the clinician programmer 1300B may include a user interface and be configured to wirelessly communicate with other components of the VNS system 1000, such as the VNS 1100, the first implantable sensor 1111A, the wearable device 1200, and/or the server 1400. The clinician programmer 1300B may be configured to receive and display data sensed by one or more sensors of the VNS system 1000 (e.g., the first implantable sensor 1111A, the second implantable sensor 1111B, and/or the external sensor 1211) and/or to control one or more settings or functions of the VNS 1100. Thus, the clinician programmer 1300B can allow a clinician to monitor various biometrics of the patient and/or to adjust the stimulation provided by the VNS 1100.

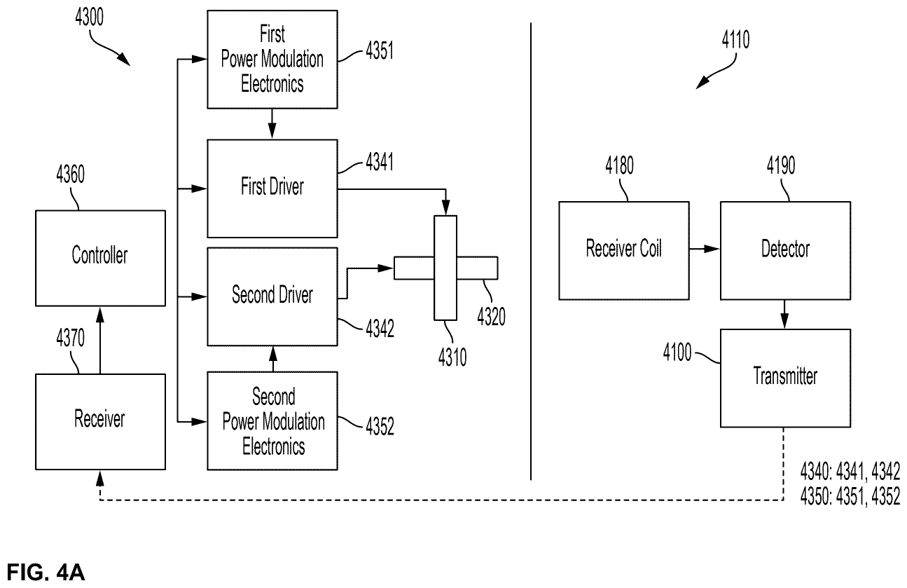

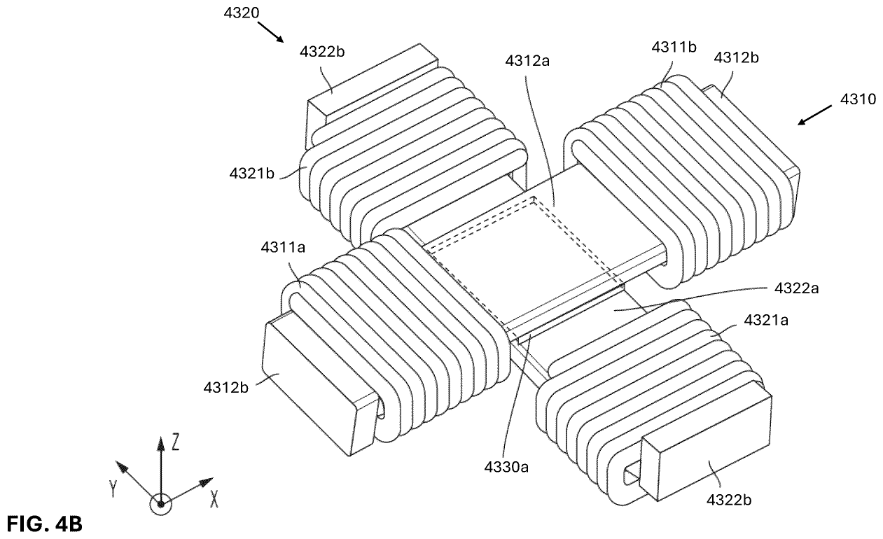

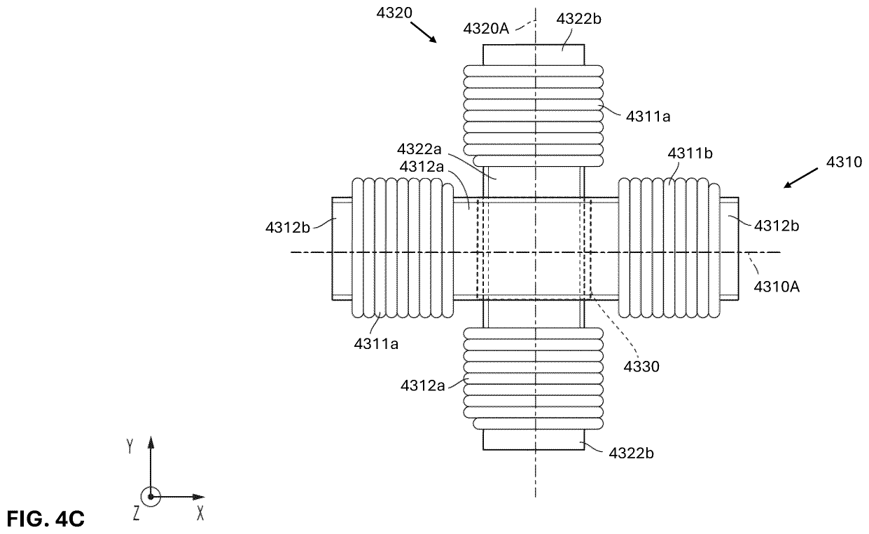

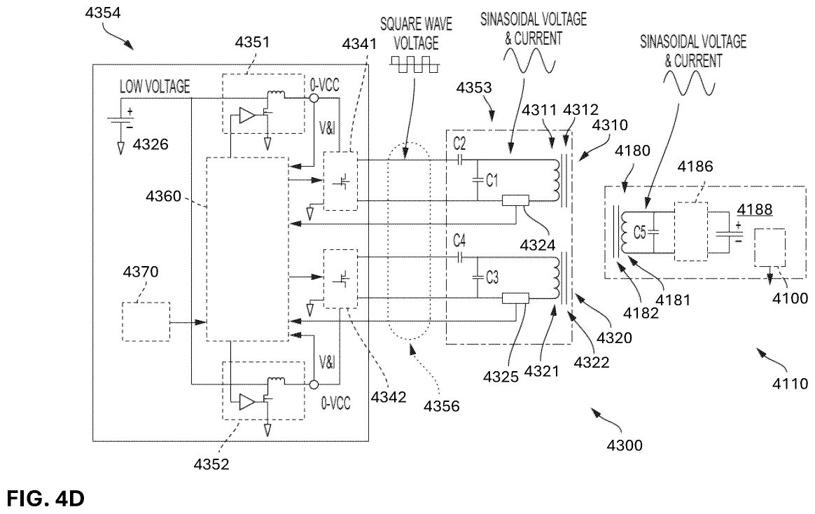

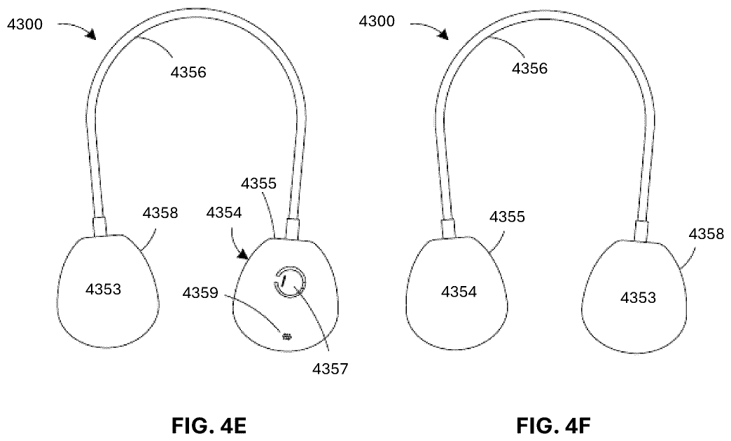

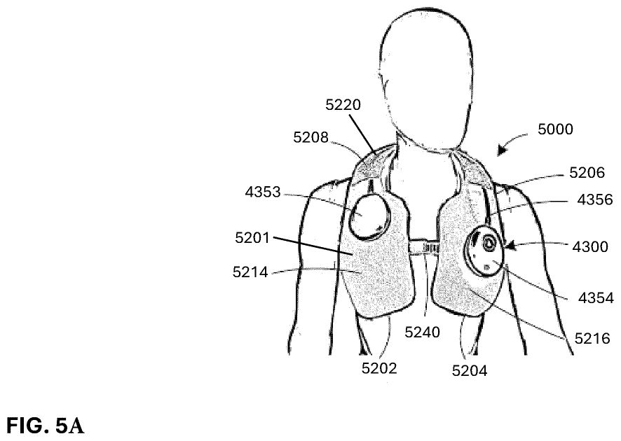

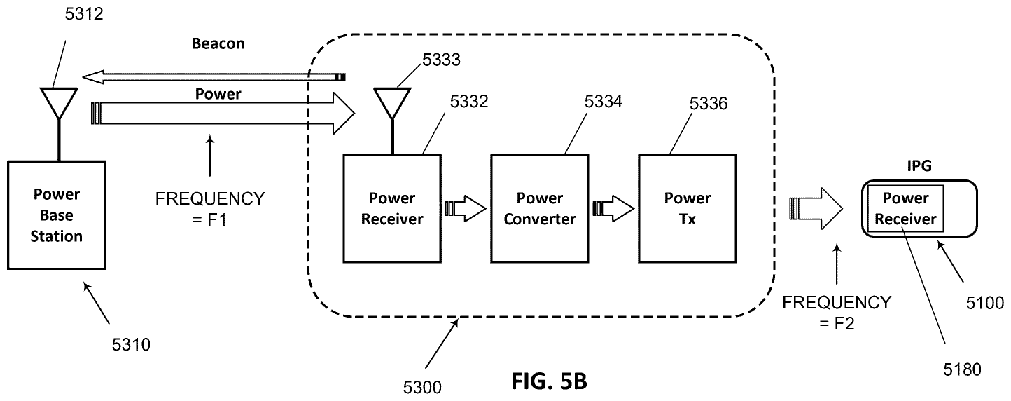

The external charger 1300C may be configured to wirelessly transmit power to the VNS 1100 and/or to other implanted devices of the VNS system 1000 (e.g., to the first implantable sensor 1111A) to charge or power the implanted components. For example, the external charger 1300 may be configured to inductively couple to one or more of the implanted devices to provide power to said device(s). In some examples, the external charger 1300C may be configured to wirelessly communicate with the server 1400, e.g., via a Wi-Fi, radio (including RF or Bluetooth), or other wireless connection. Additional details, features, and examples of external chargers will be described with reference to FIGS. 4A-5.

The server 1400 may provide various functions in the VNS system 1000. In some examples, the server 1400 includes a cloud-based server so that one or more of such functions can be performed in a cloud-based environment. The server 1400 may be configured to wirelessly communicate with (e.g., receive information from and/or transmit information to) other components of the VNS system 1000, such as the VNS 1100, the first implantable sensor 1111A, the wearable device 1200, other external sensors, and/or one or more of the external electronic devices 1300A, 1300B, and 1300C. The server 1400 may be configured to function as a communication intermediary between any two or more components of the VNS system 1000.

In some examples, the server 1400 is configured to store data and/or perform data processing. For example, the server 1400 may be configured to store biometric data sensed by any combination of the sensors of the VNS system 1000. In some examples, the server 1400 is configured to process (e.g., analyze) the sensed biometric data, determine and transmit instructions regarding the stimulation to be provided by the VNS 1100 (e.g., determine one or more stimulation parameters and/or a stimulation mode), and/or transmit other notifications regarding the patient (e.g., to the patient's physician, caretaker, family members, and/or emergency services). For example, the server 1400 may be configured to determine the one or more stimulation parameters and/or stimulation mode via one or more stimulation algorithms and based on the sensed biometric data. In some examples, the server 1400 may be configured to monitor, based on the sensed biometric data, the patient's vitals, emergency events (e.g., a seizure), and/or biological or medical responses of the patient in response to the stimulation provided by the VNS 1100.

Generally, any data within the scope of the present disclosure can be stored on the server 1400, and any processes within the scope of the present disclosure can be performed by the server 1400. Storing and processing data by the server 1400, instead of by the VNS 1100, the wearable device 1200, or other components of the VNS system 1000 capable of processing data, can advantageously reduce the processing load and improve energy efficiency of the other components of the VNS system 1000. For example, various processes and functions will be discussed below regarding selecting one or more sensors for sensing data, analyzing sensed data, recalibrating sensors, charging implantable components, generating and using personalized ictal models to detect seizures, vagus nerve stimulation titration, and detecting and using patient fall events in seizure detection. Any such processes and functions that are carried out by a controller may be performed by a controller of the server 1400 (e.g., after any necessary data, such as sensed data, is transmitted to the server 1400) to reduce energy consumption and processing load on other controllers of the VNS system 1000. Also, any such processes and functions may be carried out by an external controller in some examples, such as a controller in an external electronic device or wearable device, instead of an implantable controller in order to reduce energy consumption and processing load of the implantable controller.

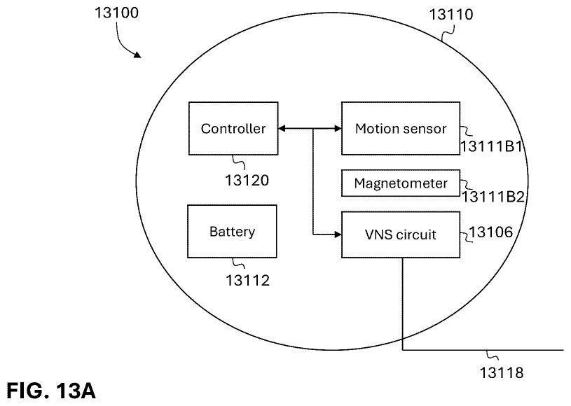

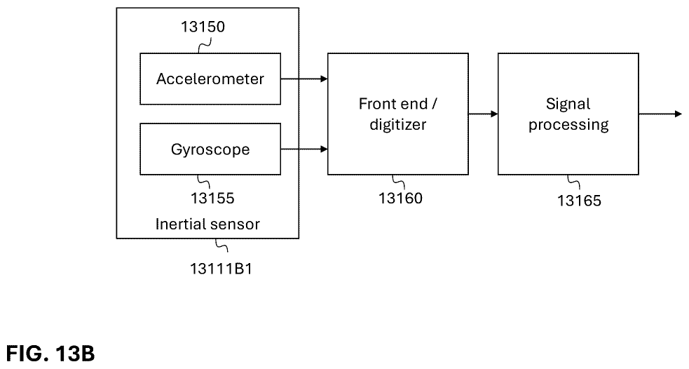

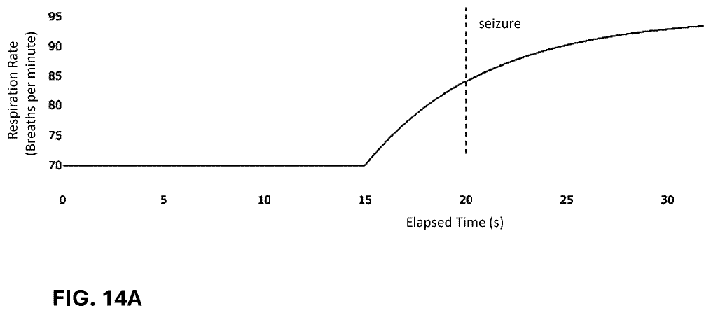

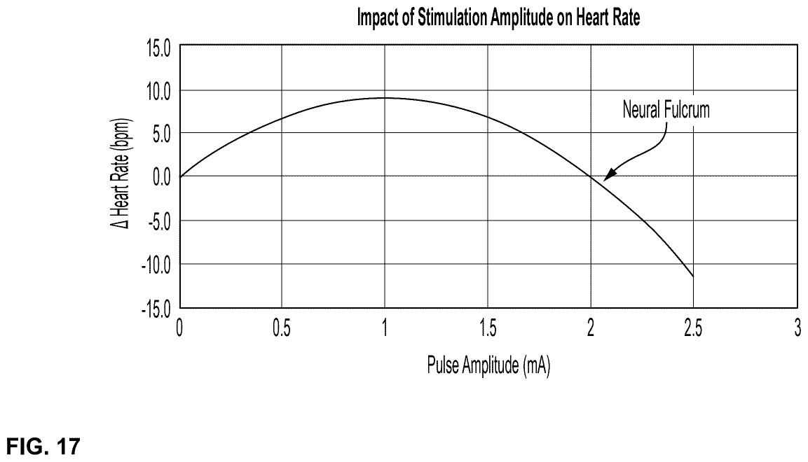

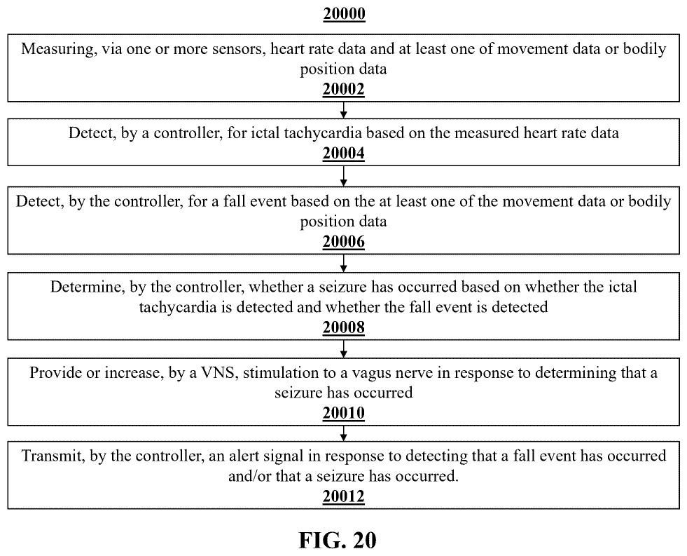

Additional details regarding the VNS system 1000 and components and processes of the VNS system 1000 will now be discussed below with reference to additional drawings. For example, additional details will be provided regarding vagus nerve stimulators with reference to FIGS. 2A-3D; regarding external chargers and charger support garments with reference to FIGS. 4A-5; regarding sensors, sensor selection, sensor recalibration, controller selection, and other components and processes of VNS systems with reference to FIGS. 6A-12; regarding generating and using personalized ictal models for seizure detection with reference to FIGS. 13A-16; regarding titration of vagus nerve stimulation with reference to FIGS. 17-19; and regarding detecting and using patient fall events to detect seizures with reference to FIG. 20. The features, processes, and details discussed herein regarding VNS systems can be combined in any manner.

### II. Vagus Nerve Stimulators

FIG. 2A is a diagram illustrating a VNS 2100 according to some examples and when subcutaneously implanted in the chest of a patient. FIG. 2B is a schematic diagram of the IPG 2110 of the VNS 2100 of FIG. 2A according to some examples.

Referring collectively to FIGS. 2A and 2B, the VNS 2100 may include an IPG 2110, a stimulation lead 2118 coupled via a connector 2130 at a proximal end of the stimulation lead to the IPG 2110, and a nerve cuff 2124 coupled to the stimulation lead 2118 at, for example, a distal end of the stimulation lead. The nerve cuff 2124 may include a plurality of electrodes 2128 wrapped circumferentially around the vagus nerve 2101. The nerve cuff 2124 may include 2, 3, 4, 5, 6, 7 or more electrodes. The nerve cuff 2124 may be configured to provide tripolar, bipolar stimulation, or monopolar (unipolar) stimulation depending on how many electrodes are activated and the polarities applied to the activated electrodes. The electrodes may be arranged to be partly circumferential around or to completely encircle the vagus nerve 2101. For example, a set of four arc-shaped or semi-circular electrodes may be placed within and evenly spaced along the circumference of a circular housing (e.g., to allow each electrode to stimulate a different quadrant of a cuffed nerve). In some examples, a single electrode or multiple electrodes within the cuff are used to deliver stimulation (functions as cathode electrodes) while, concurrently, a different subset of one or more electrodes function as return electrode(s) (e.g., as an indifferent electrode or an anode). In some examples, the IPG 2110 may have a housing 2102 (e.g., a conductive CAN) that is made at least partly of a metal, such as titanium alloy, which is electrically conductive and which can be selected or programmed to function as a return electrode (e.g., anode electrode or indifferent electrode) electrode. In some examples, the housing 2102 is hermetically sealed and may enclose at least some of the electronic components of the IPG 2110. A controller 220 of the IPG 2110 may be programmed to select the housing 2102 as inactive, in which case stimulation at the nerve cuff 2124 can be operated in a bipolar stimulation mode—that is, at least one electrode in the cuff must be active as a cathode delivering electrical stimulation to a target nerve, while concurrently, at least one electrode in the nerve cuff 2124 is selected and operating as an anode. If the metal portion of the housing 2102 is programmed to function as a return, indifferent anode, then at least one of the electrodes in the nerve cuff 2124 can be active and function as a cathode to deliver electrical stimulation to the target nerve. This latter mode of stimulation is called unipolar or monopolar stimulation if the only active electrode(s) in the nerve cuff 2124 are cathode(s). In some examples, the IPG 2110 is configured to activate only some of the electrodes and to not activate the remaining electrodes. For example, the IPG 2110 may be configured to maintain one or more of the electrodes on the nerve cuff 2124 in an inactive state, meaning they do not function as either a cathode or an anode.

The IPG 2110 may also include a rechargeable battery 2112, which may be rechargeable inductively through the housing 2102 and through intact patient skin, i.e., transcutaneously. In some examples, the IPG 2110 may include a primary battery (a single-use, non-rechargeable, battery). The IPG 2110 may include a header 2119 attached to the housing 2102 and including a lead receptacle 2119a configured to receive the connector 2130 of the stimulation lead 2118. The connector 2130 may include a plurality of connector contacts that are respectively electrically connected to the electrodes on the nerve cuff 2124 through the lead body 2104 of the stimulation lead 2118. The header 2119 may include a plurality of electrical connections (e.g., feedthrough pins) that extend from stimulation circuitry of the IPG 2110 (e.g., a pulse generator 2106) to the lead receptacle 2119a and are configured to respectively electrically connect with the plurality of connector contacts of the connector 2130 when the connector 2130 is inserted into the lead receptacle 2119a. In this manner, stimulation (e.g., electrical current) can be provided from the stimulation circuitry of the IPG 2110 to the electrodes of the nerve cuff 2124.

The controller 2120 may include processing circuitry configured to control the transmission and/or stimulation parameters (e.g., the stimulation current amplitude, stimulation pulse width, and/or stimulation frequency) of electrical stimulation provided by the IPG 2110, as described in greater detail herein. For example, with bipolar stimulation, the controller 2120 may be configured to classify (e.g., activate) each of one or more electrodes in the nerve cuff 2124 as a cathode and to classify each of one or more other electrodes in the nerve cuff 2124 as an anode. The controller 2120 can then activate stimulation of the one or more cathodes while the one or more anode electrodes are concurrently used as a return. In some examples, the classification of each electrode may be based on settings stored in a memory 2114 and/or received as instructions from an external component, such as the patient remote 1300A, the clinician programmer 1300B, or the server 1400 (see FIG. 1).

The VNS stimulator 204 may include a rechargeable battery 212 that can be accessible for recharging inductively. In some examples the battery 212 may be a single-use, primary cell battery. The battery 212 of VNS stimulator 204 may be configured to supply power to a pulse generator 206, which may be programmed to generate a periodic electrical pulse having a set frequency and pulse width. The pulse generator can be activated and deactivated (e.g., deactivate, or cease, delivery of the stimulation pulses to the electrode or electrodes to be turned off) via a switch 221. The connections on the integrated circuits may be coupled together selectively via a small printed circuit board 208. In other examples, the VNS stimulator 204 may be implemented as a SoC on a die, or a packaged die.

The IPG 2110 may further include a transceiver 2116 (or a receiver or a transmitter). In some examples, the transceiver 2116 includes a wireless receiver configured to receive wireless signals, e.g., Bluetooth Low Energy, from a source external to the patient. In some examples, the transceiver 2116 may further include a wireless transmitter (e.g., a Bluetooth Low Energy radio) configured to wirelessly transmit signals. The wireless transmitter may allow the IPG 2110 to, for example, provide information or feedback to another component of the VNS system, such as to the wearable device 1200, the patient remove 1300A, the clinician programmer 1300B, and/or the server 1400. The wireless receiver may allow the IPG 2110 to receive instructions for the controller 2120 to modify one or more stimulation parameters and/or titrate a stimulation pulse.

Titrating a stimulation pulse may include adjusting one or more of the stimulation parameters: pulse amplitude, pulse frequency, and/or pulse width to a value or values to yield a desired therapy. Titration as a general term may also include (a) selecting an electrode or electrodes as a cathode, and an electrode as an anode (for bipolar stimulation) or (b) selecting an electrode or electrodes as a cathode, and the housing 2102 as the indifferent, return anode (for unipolar stimulation). In the case of treating epilepsy, successfully titrating the stimuli delivered to the vagus nerve can prevent seizures before their onset or, if a seizure occurs, will reduce the severity and duration of the seizure. Titration of the stimulation pulses can be performed to find the stimulation parameters that stimulate the target nerve fibers that provides efficacious response, for example, with epilepsy to prevent or reduce the severity or number of occurrences of epileptic seizures. In another treatment example for titrating the stimulation pulses to treat chronic depression, the stimulation parameters are selected to eliminate or reduce the severity of depression.

The wireless transceiver 2116 may be configured to receive information including events recorded or detected by an implantable sensor (e.g., the first implantable sensor 1111A) or from one or more external sensors (e.g., the external sensor 1211). When acting as a transmitter, the transceiver 2116 can provide feedback signals to external sources using data generated by controller 2120. In some examples, the information received from the wireless transceiver 2116 may be provided to a memory 214. The controller 2120 may access the memory 2114 to receive and process instructions to deliver electrical pulses to one or more electrode(s) of the nerve cuff 2124, or to temporarily deactivate stimuli delivered by the pulse generator 2106.

In some examples, the IPG 2110 may further include an inertial measurement unit (“IMU”) to measure heart rate and/or heart rate variability (HRV). In some examples, the IMU may be configured to additionally or alternatively measure other physiologic parameters, such as respiration, patient movement, and/or patient position.

FIG. 3A depicts a VNS 3100, according to some examples, and schematically depicts certain external electronic devices. FIG. 3B depicts the stimulation lead 3118 and nerve cuff 3124 of the VNS 3100 of FIG. 3A according to some examples. FIG. 3C is an end view of the nerve cuff 3124 of the VNS 3100 of FIG. 3B when the nerve cuff 3124 is in a furled state, according to some examples. FIG. 3D is a schematic view of the IPG 3110 of the VNS 3100 of FIG. 3A according to some examples. The VNS 3100 may include features similar to, or the same as, features of the VNS 2100 of FIGS. 2A-2B.

Referring collectively to FIGS. 3A-3D, the VNS 3100 may include an IPG 3110, a stimulation lead 3118 coupled via a connector 3130 to the IPG 3110, and a nerve cuff 3124 at a distal end of the stimulation lead 3118. The nerve cuff 3124 includes a nerve cuff body 3126 configured to be flexible and to at least partly encircle the vagus nerve, and a plurality (six in this example) of electrodes 3128a-3128f on (e.g., affixed to) an inside of the nerve cuff body 3126 and arranged such that at least some of the electrodes 3128a-3128f at least partly circumferentially surround, and are in contact with, the vagus nerve. The connector 3130 at the proximal end of the stimulation lead 3118 includes a plurality (six in this example) of connector contacts 3132a-3132f that are respectively electrically coupled to the electrodes 3128a-3128f on the nerve cuff 3124 through a lead body 3104 and the nerve cuff body 3126. In some examples, the respective electrical connections between the connector contacts 3132a-3132f and the electrodes 3128a-3128f are electrically insulated from each other within the lead body 3104 and the nerve cuff body 3126.

The IPG 3110 may include a housing 3102 configured to contain at least some of the electronic components of the IPG 3110 and, in some examples, configured to be activated as an electrode (e.g., as a return anode) during electrical stimulation. The IPG 3110 may include a header 3119 attached to the housing 3102 and having a lead receptacle 3119a configured to receive the connector 3130 of the stimulation lead 3118. The header 3119 may include a plurality of feedthrough pins electrically coupled between stimulation circuitry 3106 of the IPG 3110 and the lead receptacle 3119a. The feedthrough pins may be configured to respectively electrically connect with the connector contacts 3132a-3132f when the connector 3130 is inserted into the lead receptacle 3119a.

The IPG 3110 may include the stimulation circuitry 3106, control circuitry 3120, a memory 3114, power circuitry 3136, a power source 3112 (e.g., a rechargeable battery), communication circuitry 3116, and sensing circuitry 3138, and these components may be mounted on a printed circuit board (PCB).

The control circuitry 3120 may be operatively coupled to the other components of the IPG 3110 and may be configured to perform operations of the IPG 3110 (e.g., to control the other components of the IPG 3110) in response to executing instructions stored in the memory 3114.

The stimulation circuitry 3106 may include a pulse generator and may be configured to controllably provide electrical stimulation (e.g., electrical current pulses) to the electrodes 3128 of the nerve cuff 3124. The stimulation circuitry 3106 may be configured to selectively activate any combination of the electrodes 3128 (and, in some examples, also the housing 3102) for providing stimulation and may selective classify each of the activated electrodes as a cathode or as an anode. For example, the stimulation circuitry 3106 may be configured to apply bipolar stimulation by activating one or more of the electrodes 3128 as cathodes and one or more others of the electrodes 3128 as anodes such that electrical current flows from the cathodic electrode(s) 3128 to the anodic electrode(s) 3128. In some other examples, the stimulation circuitry 3106 may be configured to apply unipolar stimulation by activating one or more of the electrodes 3128 as cathodes and activating the housing 3102 as an anode so that electrical current flows from the cathodic electrodes 3128 to the anodic housing 3102.

The communication circuitry 3116 may be configured for wirelessly communicating transcutaneously (through the patient's skin) with external components, such as the server 1400, the patient remote 1300A, the clinician programmer 1300B, and/or the wearable device 1200 using, for example, radio frequency (RF) signals. The communication circuitry 72 may include one or more AC coils for transmitting and receiving the RF signals to and from the external component(s).

The rechargeable battery 3112 and power circuitry 3136 may be configured for providing operating power to the IPG 3110. The rechargeable battery 3112 may include, for example, a lithium-ion or lithium-ion polymer battery, and may be configured to provide an unregulated voltage to the power circuitry 3136. The power circuitry 3136 may be configured to generate regulated or unregulated voltage to the various circuits located within the IPG 3110. For example, the power circuitry 3136 may be configured to provide the voltage to the stimulation circuitry 3106, which may then generate the stimulation current based on the received voltage. The rechargeable battery 3112 may be configured to be inductively recharged. For example, the IPG 3110 may include a receiver coil, configured to generate an AC current in response to receiving an alternating magnetic field from the external charger 1300C, and a rectifier configured to rectify the AC current into a DC current and to use the DC current to recharge the rechargeable battery 3112 (or, in some examples, to power components of the IPG 3110 directly without first storing the DC current in a battery). As discussed in more detail below, the external charger 1300C may be configured to generate the alternating magnetic field to power or charge the IPG 3110 and, in some examples, may be configured to establish an RF (BLE) link with the IPG 3110. In some examples, the receiver coil may be one of the coils of, or coupled to, the communication circuitry 3116 for receiving and/or transmitting the communication signals.

The sensing circuitry 3138 may have features similar to, or the same as, the second implantable sensor 1111B of the VNS 1100 of FIG. 1. For example, the sensing circuitry 3138 may include one or more sensors (e.g., an IMU and/or an accelerometer) configured to sense (e.g., measure) biometric data (e.g., physiological parameters and biomarkers), which may include at least one of heart rate, heart rate variability, respiration, patient movement (e.g., patient activity and/or mobility), patient position, blood pressure, or oxygen saturation (SpO2). The one or more sensors may be at least partly contained within the housing 3102 and, in some examples, be exposed to the exterior of the housing 3102. The sensing circuitry 3138 may be configured to transmit the sensed data to the control circuitry 3120, which may store the sensed data in the memory 3114, transmit the sensed data via the communication circuitry 3116 to an external component (e.g., the server 1400, the patient remote 1300A, or the clinician programmer 1300B), and/or process (e.g., analyze) the sensed data and adjust the stimulation provided by the stimulation circuitry 3106 based on the sensed data.

### III. External Charger

FIG. 4A is a schematic view of some components of an external charger 4300 and of an IPG 4110 according to some examples. FIG. 4B is a perspective view of first and second transmitting coils 4310 and 4320 of the external charger 4300 of FIG. 4A according to some examples, and FIG. 4C is a top-down view of the first and second transmitting coils 4310 and 4320 of FIG. 4B. FIG. 4D is another schematic view of some components of the external charger 4300 and of the IPG 4110 according to some examples.

Referring concurrently to FIGS. 4A-4D, the external charger 4300 may include the first transmitting coil 4310, the second transmitting coil 4320 positioned on (e.g., above) the first transmitting coil 4310, a driver 4340 configured to drive the first transmitting coil 4310 with a first AC current and the second transmitting coil 4320 with a second AC current, power modulation electronics 4350 configured to modulate the first and second AC currents provided by the driver 4340, a controller 4360 (e.g., a microcontroller) operatively coupled to, and configured to control operations of, the driver 4340 and the power modulation electronics 4350, a receiver 4370 for receiving information (e.g., information transmitted by the IPG 4110), and a memory (not shown) storing computer readable instructions to be executed by the controller 4360 to perform the operations of the controller 4360 and/or configured to store data (e.g., the information received via the receiver 4370).

The IPG 4110 may include a receiver coil 4180, a detector 4190 configured to detect information about power received in the receiver coil 4180, and a transmitter 4100 configured to transmit information (e.g., transmit information to the external charger 4300). In some examples, the transmitter 4100 may be a radio or an RF transmitter.

The external charger 4300 may be configured to generate an oscillating magnetic field by driving the first and second transmitting coils 4310 and 4320 with the first and second AC currents, respectively, and to rotate the direction of the magnetic field by controlling (e.g., setting or adjusting) a first magnitude of the first AC current, a second magnitude of the second AC current, and a phase difference between the first and second AC currents. For example, the external charger 4300 may be configured to differentially drive the first and second transmitting coils 4310 and 4320 (e.g., by differentially setting the amplitudes and/or phases of the first and second AC currents) to, for example, rotate the direction of the generated magnetic field. When the external charger 4300 generates the magnetic field and the IPG 4110 is in the proximity to the external charger 4300, a current may be generated in the receiver coil 4180 by electromagnetic induction (e.g., wireless resonant induction). The detector 4190 may be configured to detect information (e.g., power, amplitude, etc.) about the current generated in the receiver coil 4180, and the transmitter 4100 may transmit (e.g., wirelessly transmit) the detected information to other components of the VNS system, such as the external charger 4300 (e.g., to the receiver 4370), the server 1400, the wearable device 1200, patient remote 1300A, and/or the clinician programmer 1300B. The controller 4360 may control the driver 4340 and the power modulation electronics 4350 based on the information received by the receiver 4370 to control the direction of the magnetic field at the receiver coil 4180. For example, the detected information can indicate how aligned (e.g., a degree of alignment) between the receiver coil 4180 and magnetic field at the receiver coil 4180, and the controller 4360 may be configured to adjust the parameters (e.g., amplitude and/or phase) of the first and second AC currents based on the detected information to change the direction of the magnetic field to increase alignment.

The first transmitting coil 4310 may include a first rod 4312 and a first wire 4311 wound around the first rod 4312, and the second transmitting coil 4320 may include a second rod 4322 and a second wire 4321 wound around the second rod 4322.

The first transmitting coil 4310 may be aligned along a first axis 4310A, and the second transmitting coil 4320 may be aligned along a second axis 4320A different from the first axis 4310A. In some examples, the second axis 4320A is substantially perpendicular (e.g., within 5 degrees, 2 degrees, or 1 degree from being exactly perpendicular) to the first axis 4310A. When the first and second axes 4310A and 4320A are substantially perpendicular, coupling between the first and second transmitting coils 4310 and 4320 may be reduced or substantially prevented. In FIGS. 2-4, the first axis 4310A is shown as being aligned along an X-axis, and the second axis 4320A is shown as being aligned along a Y-axis. The X-, Y-, and Z-axes in FIGS. 4B and 4C form a mutually orthogonal coordinate system.

The second transmitting coil 4320 may be on (e.g., above) the first transmitting coil 4310 and may overlap the first transmitting coil 4310 in a plan view (shown in FIG. 4C) at an area of overlap 4330. In some examples, the area of overlap 4330 corresponds to a center region of the first transmitting coil 4310 and a center region of the second transmitting coil 4320. The second transmitting coil 4320 may be spaced apart from the first transmitting coil 4310 in a thickness direction (e.g., along the Z-axis direction) at the area of overlap 4330.

An intermediate space 4330a between the first and second transmitting coils 4310 and 4320 in the area of overlap 4330 may include (e.g., be filled or at least partially filled with) a nonmagnetic material having a low permeability, for example, air, plastic, foam, one or more non-ferrimagnetic materials, one or more low permeability metals (e.g., aluminum and/or copper), etc. In some examples, when the intermediate space 4330a is filled with air, a frame or housing may be utilized to hold the first and second transmitting coils 4310 and 4320 and/or to maintain the relative positions of the first and second transmitting coils 4310 and 4320 with respect to each other. In some examples, the material in the intermediate space 4330a has a relative permeability equal to or less than about 5, for example, in the range of about 1 to about 1.5. In some examples, the material in the intermediate space 4330a may be diamagnetic (e.g., a material having a relative permeability in the range of about 0 to about 1). Therefore, in some examples, the second transmitting coil 4320 does not contact the first transmitting coil 4310, and the first and second transmitting coils 4310 and 4320 are magnetically independent (e.g., magnetically decoupled and/or magnetically isolated from each other) and/or electrically independent (e.g., electrically decoupled and/or electrically isolated) from each other. Because the first and second transmitting coils 4310 and 4320 are not in contact, coupling between the first and second transmitting coils 4310 and 4320 may be reduced or substantially prevented. That is, the first transmitting coil 4310 may generate a first magnetic field without being significantly influenced by the presence of the second transmitting coil 4320, and the second transmitting coil 4320 may generate a second magnetic field without being significantly influenced by the presence of the first transmitting coil 4310. A magnetic field generated by the external charger 4300 may be a superposition of the first and second magnetic fields generated by the first and second transmitting coils 4310 and 4320, respectively.

The first rod 4312 may include a magnetic material having a high permeability, such as a ferrimagnetic material (e.g., soft ferrite material), such as nickel- or manganese-based ferrites (e.g., MnZn, NiZn, and/or the like). The magnetic material may increase the intensity of a magnetic field generated by the first transmitting coil 4310. In some examples, the material of the first rod 4312 may have a relative permeability equal to or greater than about 5, for example, in the range of about 4300 to about 4300,000. The second rod 4322 may include any material that the first rod 4312 may include, and the second rod 4322 may include a material that is the same as, or different from, a material included in the first rod 4312. In some examples, a ratio of the permeability of a material in the first rod 4312 to the permeability of the material in the intermediate space 4330a may be equal to or greater than approximately (about) 5. When the permeability of the materials of the first and second rods 4312 and 4322 are significantly larger than the permeability of the material in the intermediate space 4330a, coupling between the first and second transmitting coils 4310 and 4320 may be reduced or substantially prevented. For example, a magnetic field flowing through the first rod 4312 may be blocked (by the material in the intermediate space 4330a) from permeating through the intermediate space 4330a and into the magnetic material of the second rod 4322. Thus, the presence of the second transmitting coil 4320 may not substantially affect the first magnetic field generated by the first transmitting coil 4310, and vice versa.

The first rod 4312 may include a first main rod 4312a and first thick portion (e.g., a tab or a flange) 4312b at an end (e.g., both ends) of the first main rod 4312a, and the second rod 4322 may include a second main rod 4322a and a second thick portion (e.g., a tab or a flange) 4322b at an end (e.g., both ends) of the second main rod 4322a. The first main rod 4312a may have any suitable shape. The second main rod 4322a may have any shape that the first main rod 4312a may have, and the shape of the second main rod 4322a may be the same as, or different from, the shape of the first main rod 4312a. In some examples, the first main rod 4312a has a cylindrical shape. In other examples, the first main rod 4312a has a rectangular shape having a length along the X-axis, a width along the Y-axis, and a thickness along the Z-axis. In some examples, the first main rod 4312a has a shape whereby the width of the first main rod 4312a is less than the length of the first main rod 4312a, and/or the thickness (e.g., along the Z-axis) of the first main rod 4312a is less than the width and/or length of the first main rod 4312a.

A thickness of the intermediate space 4330a may be relatively small compared to the dimensions of the first and second transmitting coils 4310 and 4320. For example, the thickness of the intermediate space 4330a may be less than the length, the width, and/or the thickness of the first main rod 4312a. Because the first and second magnetic fields generated by the first and second transmitting coils 4310 and 4320 will each generally decrease in magnitude as respective distances from the first and second transmitting coils 4310 and 4320 increase, it is advantageous for the thickness of the intermediate space 4330a to be small in order to minimize or at least reduce a disparity between a distance between the IPG 4110 and the first transmitting coil 4310 and a distance between the IPG 4110 and the second transmitting coil 4320. When the disparity is large, one of the first and second transmitting coils 4310 and 4320 may have an unintended disproportionate effect on the IPG 4110 compared to the other one of the first and second transmitting coils 4310 and 4320. Accordingly, in one or more examples, the thickness of the intermediate space 4330a may be sufficiently small such that the first and second transmitting coils 4310 and 4320 are substantially coplanar to advantageously minimize or at least reduce the disproportionate effect of one of the first and second transmitting coils 4310 and 4320 on the IPG 4110.

In some examples, a thickness of the first main rod 4312a at the area of overlap 4330 is less than a thickness of the first main rod 4312a at an area outside of the area of overlap 4330. For example, the first main rod 4312a may have an indent or recess (e.g., a step) at the area of overlap 4330 that faces the second main rod 4322a. When one or both of the first and second main rods 4312a and 4322a have such an indent or recess, the distance between the first and second transmitting coils 4310 and 4320 may be reduced. In some examples, the indent or recess in one or both of the first and second main rods 4312a and 4322a may allow the first and second wires 4311 and 4321 to be coplanar (or substantially coplanar).

The first thick portion 4312b may be at an end (or end portion) of the first main rod 4312a, and a thickness (e.g., along the Z-axis direction) of the first thick portion 4312b may be greater than a thickness of the first main rod 4312a. For example, as shown in FIG. 4B, the first thick portion 4312b may protrude toward the second transmitting coil 4320. Similarly, the second thick portion 4322b may be at an end (or end portion) of the second main rod 4322a, and a thickness of the second thick portion 4322b may be greater than a thickness of the second main rod 4322a. For example, the second thick portion 4322b may protrude toward the first transmitting coil 4310. For example, the second thick portion 4322b of the second transmitting coil 4320 may protrude in a direction opposite to a protruding direction of the first thick portion 4312b of the first transmitting coil 4310. Because the first and second thick portions 4312b and 4322b of the first and second transmitting coils 4310 and 4320 may protrude toward the second and first transmitting coils 4320 and 4310, respectively, the distance along the Z-axis direction between the ends of the first rod 4312 and the ends of the second rod 4322 may be reduced or eliminated, and thus, the ends of the first and second rods 4312 and 4322 may be substantially coplanar.

The first wire 4311 may be wound around the first rod 4312 in any suitable configuration. The second wire 4321 may be wound around the second rod 4322 in any configuration that the first wire 4311 may be wound around the first rod 4312. In some examples, the first wire 4311 is wound around the first main rod 4312a and is not wound around the first thick portion 4312b. The first wire 4311 may be wound around substantially the entire length of the first main rod 4312a. For example, the first wire 4311 and the first main rod 4312a may form a solenoid. In some examples, the first wire 4311 is wound around two ends (or two end portions) of the first main rod 4312a to form first and second sub-coils 4311a and 4311b at the two ends (or two end portions) of the first main rod 4312a, and the first wire 4311 exposes, and is not wound around, a portion (e.g., an exposed intermediate or central portion) of the first main rod 4312a between the first and second sub-coils 4311a and 4311b. The exposed portion of the first main rod 4312a may include a portion of the first main rod 4312a corresponding to the area of overlap 4330 between the first and second transmitting coils 4310 and 4320. When the first wire 4311 is not wound around the first main rod 4312a at the area of overlap 4330, the thickness of the first transmitting coil 4310 at the area of overlap 4330 may be reduced. The first sub-coil 4311a may be electrically coupled (e.g., electrically connected) to the second sub-coil 4311b in series or in parallel. In some examples, the first sub-coil 4311a is not electrically coupled (e.g., electrically connected) to the second sub-coil 4311b, and the first and second sub-coils 4311a and 4311b are separately driven (e.g., synchronously driven in phase).

The external charger 4300 may generate a magnetic field by driving the first AC current through the first wire 4311 and/or driving the second AC current through the second wire 4321. The first and second AC currents may be driven in phase (i.e., with substantially 0° phase difference between the first and second AC currents) or substantially 180° out of phase.

In some examples, the external charger 4300 is configured to generate a continuously rotating magnetic field. The external charger 4300 may be configured to cause the generated magnetic field to continuously rotate by respectively providing the first and second transmitting coils 4310 and 4320 with equal first and second currents that are equal in frequency and that have a phase difference substantially equal to 90 degrees (e.g., within 10, 5, 3, 2, 1, or 0.5 degrees of 90 degrees).

Referring to FIG. 4D, the external charger 4300 may include a transmission assembly 4353, an electronics assembly 4354, and a cable 4356 coupled between the transmission assembly 4353 and the electronics assembly 4354.

The transmission assembly 4353 may include the first and second transmitting coils 4310 and 4320, a first capacitor C1 coupled in parallel with the first wire 4311 (e.g., a first winding) of the first transmitting coil 4310, a second capacitor C2 coupled in series with the first capacitor C1 and first wire 4311, a third capacitor C3 coupled in parallel with the second wire 4321 (e.g., a second winding) of the second transmitting coil 4320, a fourth capacitor C4 coupled in series with the third capacitor C3 and second wire 4321, a first current sensor 4324 coupled to the first wire 4311, and a second current sensor 4325 coupled to the second wire 4321.

The external charger 4300 may include a first LC resonant circuit corresponding to the first transmitting coil 4310 and a second LC resonant circuit corresponding to the second transmitting coil 4320. The first LC resonant circuit includes the first wire 4311, the first capacitor C1, and the second capacitor C2, and the second LC resonant circuit includes the second wire 4321, the third capacitor C3, and the fourth capacitor C4. In some examples, the first LC resonant circuit includes the first wire 4311 and the first capacitor C1, and the second capacitor C2 is not included, and/or the second LC resonant circuit includes the second wire 4321 and the third capacitor C3, and the fourth capacitor C4 is not included.

The first and second capacitors C1 and C2 may form an impedance matching network that matches the impedance of the first driver 4341. The resonant frequency of the first LC resonant circuit may be defined by equation 1 below,

$${f}_{res 1} = \frac{1}{2 π \sqrt{{L}_{110} ( C {1}_{cap} + C {2}_{cap} )}} , \qquad [ Equation 1 ]$$

wherein fᵣₑₛ₁ is the resonant frequency of the first LC resonant circuit, L₁₁₀ is the inductance of the first wire 4311, and C1_{cap} and C2_{cap} are the first and second capacitances of the first and second capacitors C1 and C2, respectively. The resonant frequency of the second LC resonant circuit may be defined in a manner similar to how the resonant frequency of the first LC resonant circuit is defined. In some examples, the resonant frequencies of the first and second LC resonant circuits are the same or similar.

A ratio of the second capacitance C2_{cap} to the first capacitance C1_{cap} may be set such that, when the first driver 4341 produces its highest pulse voltage, the IPG 4110 will receive its required power dependent on a coupling coefficient between the IPG 4110 and the external charger 4300.

The first and second current sensors 4324 and 4325 are respectively configured to measure or sense a current flowing through the first and second wires 4311 and 4321 and to provide information (e.g., as an analog signal) about the current to the controller 4360.

The electronics assembly 4354 may include a permanent or rechargeable battery 4326. The controller 4360 may be configured to output analog or digital signals to the first and second power modulation electronics 4351 and 4352 and to output digital square wave signals to the first and second drivers 4341 and 4342.

The controller 4360 may be configured to control the respective frequencies, the respective amplitudes, and the relative phase difference between, the first and second AC currents respectively provided to the first and second wires 4311 and 4321 via the signals that it sends to the first and second power modulation electronics 4351 and 4352 and/or the signals that it sends to the first and second drivers 4341 and 4342. The controller 4360 may receive signals (e.g., voltage feedback) from the outputs of the first and second power modulation electronics 4351 and 4352, the first and second current sensors 4324 and 4325, and the receiver 4370. The controller 4360 may be configured to control the signals that it sends to the first and second power modulation electronics 4351 and 4352 and/or the signals that it sends to the first and second drivers 4341 and 4342 based on these received signals.

The first and second drivers 4341 and 4342 may output square wave voltages to the first and second LC resonant circuits. Sinusoidal voltage and current may be provided to the first and second wires 4311 and 4321 respectively in the first and second LC resonant circuits. Local feedback information may be provided to the controller 4360 from the first and second current sensors 4324 and 4325, and global feedback information may be provided to the controller 4360 from the receiver 4370 that receives the global feedback information from the IPG 4110 (e.g., from the transmitter 4100). The local and/or global feedback information may be utilized in a feedback system.

For example, the information received from the IPG 4110 may be used to increase or decrease the respective amplitudes of the first and second currents provided to the first and second wires (or “coils”) 4311 and 4321 to adjust the power received in the IPG 4110. This may be advantageous, for example, when it is determined based on information obtained from the IPG 4110 during power transfer that a higher or lower power transfer is desired.

The IPG 4110 may include the receiver coil 4180 including a receiver rod 4182 and a winding 4181 wound around the receiver rod 4182. The receiver rod 4182 may include any material that the first rod 4312 may include, and the receiver rod 4182 may include a same or different material as the first rod 4312 includes. The winding 4181 may be wound around the receiver rod 4182, for example, to form a solenoid.

The IPG 4110 may include a fifth capacitor C5 coupled in parallel with the winding 4181 to form at least part of a third LC resonant circuit, a rechargeable battery 4188, a power management system 4186, and the transmitter 4100. In some examples, the third LC resonant circuit may include a sixth capacitor coupled in series with the fifth capacitor C5 and winding 4181. The fifth capacitor C5 and the sixth capacitor may form at least part of a capacitive matching network.

The power management system 4186 may be configured to regulate system voltage and charging of the rechargeable battery 4188, and may be configured to convert an AC voltage in the third LC resonant circuit to a DC voltage. In some examples, the IPG 4110 does not include the rechargeable battery 4188. For example, the power management system 4186 may be configured to provide the DC voltage to (e.g., directly to) electrodes or coils to provide stimulation (e.g., provide stimulation to biological tissue) and/or to perform other operations of the IPG 4110 without storing the energy of the DC voltage in a battery.

The transmitter 4100 may transmit information about a state of charge of the rechargeable battery 4188, a voltage of the rechargeable battery 4188, a rate of recharging of the rechargeable battery 4188, and/or a rectified voltage of the receiver coil 4180 and/or of the third LC resonant circuit. As described above, the controller 4360 may be configured to control the first and second AC currents respectively provided to the first and second coils 4310 and 4320 based on this information received from the IPG 4110. For example, the controller 4360 may initiate or terminate driving of the first and second coils 4310 and 4320 or may increase or decrease respective amplitudes of the first and second AC currents to adjust the power transferred to the IPG 4110.

As explained above, the external charger 4300 may be configured to generate a rotating magnetic field. To generate the rotating magnetic field, the first and second drivers 4341 and 4342 may be configured to provide the first and second equal AC currents to be equal in frequency and to have a phase difference substantially equal to 90 degrees. Under these conditions, the generated magnetic field can rotate through a full cycle at the same frequency of the first and second currents.

Energy stored in the third LC resonant circuit may oscillate (at a resonant frequency of the third LC resonant circuit). If the frequency of the first and second AC currents (and thus the frequency at which the rotating magnetic field rotates through cycles) is set to be equal to the resonant frequency of the third LC resonant circuit, then energy can be added from the generated rotating magnetic field to the energy in the third LC resonant circuit every time the rotating magnetic field rotates through a full cycle. This can occur independently of how the receiver coil 4180 is oriented relative to the first and second transmitting coils 4310 and 4320. Thus, the external charger 4300 can effectively and reliably transfer power to the IPG 4110 without needing to pre-align or actively align the generated magnetic field with the receiver coil 4180.

The external charger 4300 may be configured to determine and control power transmission levels (e.g., via a control algorithm and a feedback process with the IPG 4110) to prevent overheating of the IPG 4110, which may include a temperature sensor. For example, the controller 4360 may be configured to reduce the amplitudes of the first and second currents to reduce power transfer in response to receiving an information signal from the IPG 4110 indicating that a temperature of the IPG 4110 is above a threshold value and/or that the received power is too high. In some examples where the external charger 4300 is configured to generate the rotating magnetic field without feedback from the IPG 4110, the complexity of the structure and/or operation of the wireless power transfer system can be reduced.

In some examples, respective amplitudes of the first and second currents may be set to be substantially equal to each other (e.g., to be within 10%, 5%, 3%, 2%, 1%, or 0.5% of each other). When the phase difference between the first and second currents is substantially 90 degrees and the respective amplitudes of the first and second currents are substantially equal to each other, then the amplitude of the rotating magnetic field can be substantially constant as it rotates through a full cycle. This can cause the amplitude of the rotating magnetic field at the receiver coil 4180 (as the magnetic field rotates through alignment with the receiver coil) to be substantially independent of the orientation of the receiver coil 4180 relative to the first and second transmitting coils 4310 and 4320. Thus, the external charger 4300 can reliably and suitably transfer power to the IPG 4110 regardless of the orientation of the receiver coil 4180 relative to the first and second transmitting coils 4310 and 4320.

FIG. 4E is a front view of the external charger 4300 according to some examples, and FIG. 4F is a rear view of the external charger 4300 according to some examples. FIG. 5A depicts a charger support garment 5000, according to some examples, on a patient and the external charger 4300 attached to the charger support garment 5000.

Referring collectively to FIGS. 4E, 4F, and 5, the external charger 4300 may include a transmission assembly 4353, an electronics assembly 4354, and a cable physically and electrically coupled between the transmission assembly 4353 and the electronics assembly 4354. The external charger 4300 may be configured as a wearable device and, in this example, is configured to be worn around a patient's neck such that the transmission assembly 4353 and the electronics assembly 4354 are respectively positioned on the two sagittal sides of the patient's chest with the cable 4356 extending around the back of the patient's neck.

The transmission assembly 4353 may include a first housing 4358 containing some of the components of the external charger 4300, the electronics assembly 4354 may include a second housing 4355 containing some other components of the external charger 4300, and the cable 4356 may be coupled between the first and second housings 4358 and 4355. In some examples, a length of the cable 4356 between the transmission assembly 4353 and the electronics assembly 4354 is within a range of 10 inches to 30 inches, and is about 21 inches in the depicted example.

The transmission assembly 4353, the electronics assembly 4354, and the cable 4356 are schematically illustrated in FIG. 4D according to some examples. For example, the transmission assembly 4353 may contain the first and second transmitting coils 4310 and 4320, the first and second current sensors 4324 and 4325, and the first to fourth capacitors C1 to C4. The electronics assembly 4354 may contain the first and second drivers 4341 and 4342, the first and second power modulation electronics 4351 and 4352, the controller 4360, the battery 4326, and the receiver 4370. However, other divisions of the components of the external charger 4300 between the transmission assembly 4353 and the electronics assembly 4354 are also possible. For example, the receiver 4370 may be positioned in the transmission assembly 4353 in some examples.

At least some of the electronic components of the external charger 4300 can be positioned in the electronics component to separate them from the first and second transmitting coils 4310 and 4320 to improve the efficiency of the power transfer from the external charger 4300 to the IPG 4110 by reducing or eliminating eddy currents that form in electronic components. The magnetic field generated by the first and second transmitting coils 4310 and 4320 can cause eddy currents to form in electrically conductive materials, such as in various electronic components of the external charger 4300. These eddy currents can remove energy from the generated magnetic field that could have otherwise been transferred to the IPG 4110. The magnitude of such eddy currents depends in part on the magnitude of the generated magnetic field at the electronic components. Because the magnitude of the generated magnetic field drops as a distance away from the first and second transmitting coils 4310 and 4320 increases, positioning some of the electronic components farther away from the first and second transmitting coils 4310 and 4320 (e.g., in the electronics assembly 4354) can reduce the magnitude of the eddy currents and improve efficiency.

The IPG 4110 may be positioned on one side of the patient's chest, such as on the patient's right side. By configuring the external charger 4300 to be worn around the patient's neck, the transmission assembly 4353 can be positioned against the patient's chest at or near the location where the IPG 4110 has been implanted. This can improve power transfer efficiency by positioning the first and second transmitting coils 4310 and 4320 closer to the receiver coil 4380 in the IPG 4110. This can also improve comfort for the patient because the patient does not need to hold the external charger 4300 while power is being transferred.

In some examples, the external charger 4300 (e.g., the electronics assembly 4354) may include a user interface 4357 (e.g., a power button, display screen such as a touch display, a speaker, and/or one or more user input devices) and an alarm 4359. The user interface 4357 may allow a user to turn on the external charger 4300 and/or interact with the external charger 4300 (e.g., to control one or more settings of the external charger 4300). The alarm 4359 may be operatively coupled to the controller 4360 and configured to provide a signal (e.g., an audible, visible and/or tactile signal) indicating that the magnetic field generated by the external charger 4300 is aligned with the receiver coil 4180 of the IPG 4110, as determined by the controller 4360 (e.g., based on the various feedback signals received by the controller as described herein). For example, the alarm may indicate that the transmission assembly 4353 is suitably positioned relative to the patient's body or that the position of the transmission assembly 4353 needs to be adjusted.

The charger support garment 5000 may form part of the VNS system (e.g., the VNS system 1000 of FIG. 1) and may include a first chest part 5202, a second chest part 5204, and a neck part 5206 coupled between the first and second chest parts 5202 and 5204. The first and second chest parts 5202 and 5204 may be configured to be positioned on the two sagittal sides (e.g., right and left sides) of the patient's chest when worn, and the neck part 5206 may be configured to be worn around the back of the patient's neck so that the first and second chest parts 5202 and 5204 are held against the patient's body. Two parts of a connector 5240 (e.g., snap-fit connector) may be respectively positioned on the first and second chest parts 5202 and 5204 and configured to lockingly engage with each other to hold together the first and second chest parts 5202 and 5204.

The charger support garment 5000 may include a lower panel 5201 that forms at least part of the first and second chest parts 5202 and 5204, and that forms a lower part of the neck part 5206. The neck part 5208 may include an upper panel 5220 that is attached to a neck portion of the lower panel 5201 associated with the neck part 5208 in a manner such that the cable 4356 can extend between the lower panel 5201 and the upper panel 5220 along the neck part 5208 between the first and second chest parts 5202 and 5204. For example, the neck portion of the lower panel 5201 may have two opposite edges extending lengthwise between the first and second chest parts 5202 and 5204, and the upper panel 5220 may be permanently attached or attachable to (e.g., attachable to and detachable from) one or both of the two opposite edges of the neck portion of the lower panel 5201. In such a case, the neck portion of the lower panel 5201 and the upper panel 5220 may form a pouch that extends between the first and second chest parts 5202 and 5204 and that is open at the first and second chest parts 5202 and 5204 so that the cable 4356 can extend through the pouch between the first and second chest parts 5202 and 5204. The neck part 5208 may provide a protective barrier between the cable 4356 and the patient's skin, which can reduce irritation from the cable 4356 rubbing directly against the back of the patient's neck, and may also hold the cable 4356 down against the patient's body, which may be useful for when the patient is in motion (e.g., jogging).

The first chest part 5202 may include a first fastener 5214 configured to attach to the transmission assembly 4353, and the second chest part 5204 may include a second fastener 5216 configured to attach to the electronics assembly 4354. In some examples, each of the first and second fasteners 5214 and 5216 may include one part of a hook-and-loop fastener. For example, each of the first and second fasteners 5214 and 5216 may independently include a surface including hooks or a surface including fasteners. Such surfaces may form at least part of an outer surface (e.g., a surface facing away from the patient) of the lower panel 5201. Each of the transmission and electronic assemblies 4353 and 4354 may independently include a surface (e.g., a rear surface configured to face the charger support garment 5000) having the corresponding second part of the hook-and-loop fastener so that the transmission and electronic assemblies 4353 and 4354 can engage with the first and second fasteners 5214 and 5216. For example, the first and second fasteners 5214 and 5216 may each include a surface with hooks, and rear surfaces of the transmission and electronic assemblies 4353 and 4354 may each include fasteners. The first and second fasteners 5214 and 5216 can allow the transmission and electronic assemblies 4353 and 4354 to be secured against the patient's body. This can desirably substantially fix the position of the transmission assembly 4353 relative to the IPG 4110, and can also improve the patient's mobility and comfort by allowing the patient to move about without causing the external charger 4300 to move about.

In some examples, the lower panel 5201 includes multiple layers, including an outer layer, which includes the hook surface or loop surface for the first and second fasteners 5214 and 5216, and an inner layer that includes a different material than the outer layer. The material or the inner layer may be configured to be more comfortable (e.g., softer or smoother) for the patient compared to the material of the outer layer.

Although the external charger 4300 has been described herein as having two transmitting coils, other examples are also contemplated, such as where the external charger 4300 includes only one transmitting coil or three or more transmitting coils.

FIG. 5B is a schematic view of a VNS system according to some examples. The VNS system of FIG. 5B includes a VNS 5100, an external charger 5300, and a power base station 5310. The VNS 5100 includes a power receiver 5180 (e.g., a receiver coil) configured to inductively receive power for storage or use in the VNS 5100. The power base station 5310 is configured to generate (e.g., via an antenna 5312, such as a phased array antenna) and directionally steer a first power beam (e.g., RF power) having a first frequency F1. The charger 5300 may be a wireless power relay device configured to receive power from the power base station 5310 and to retransmit the received power to the VNS 5100 at different frequency than the frequency at which the charger puck 5300 received the power. In some examples, the charger 5300 may be configured to retransmit the received power without storing the received power in a battery. For example, the charger 5300 may not include a battery. The charger 5300 may include a power receiver 5332, a power converter 5334, a power transmitter 5336, and an antenna 5333. In some examples, the antenna 5333 may be integrated with the power receiver 5332.

The charger 5300 may be configured to transmit, via the antenna 533, a beacon signal to indicate to the power base station 5310 the location of the charger 5300, and the power base station 5310 may be configured to control the direction of the first power beam based on the beacon signal so that the first power beam is transmitted to the charger 5300. The charger 5300 may be configured to receive the first power beam having the first frequency F1 via the power receiver 5332 (e.g., via the antenna 5333 or a separate antenna). The power converter 5334 may be configured to convert the received power into a form having a second frequency F2 different than (e.g., greater than or less than) the first frequency F1, and the power transmitter 5336 may transmit the converted power (e.g., as an alternating magnetic field) having the second frequency F2 to the power receiver 5180. In some examples, the first frequency F1 may be in the range of 1 MHz to 300 GHz, and the second frequency F2 may be in the range of 1 kHz to 1 MHz.

As an example, the charger 5300 may include a receiver antenna, an AC to DC converter, a transmitter coil, and a coil driver. The receiver antenna may be configured to receive RF power, having the first frequency F1, from the power base station 5310 and to convert the RF power into a first AC current having the first frequency F1. The AC to DC converter (e.g., a rectifier) may be configured to convert the first AC current into a DC current. The coil driver may be configured to receive the DC current and to generate a second AC current, having the second frequency F2, based on the DC current. The coil driver may be configured to drive the transmitter coil with the second AC current to generate an alternating magnetic field in the vicinity of the power receiver 5180.

As explained above, the charger 5300 may not include a battery. It may be configured to be small, light, and comfortable for a patient to wear on his or her person. In some examples, the charger 5300 may include a biocompatible adhesive configured to attach the charger 5300 onto the skin of the patient. In some other examples, the charger 5300 may be configured to be attached (e.g., via a hook-and-loop fastener, adhesive, etc.) to patient's clothing.

### IV. Biometric Sensing Using Implantable and External Sensors

FIG. 6A schematically illustrates a VNS system 6000 according to some examples. The VNS system 6000 may be an example of the VNS system 1000 of FIG. 1 and, thus, may have features similar to, or the same as, the features of the VNS system 1000 that may not be repeated. The VNS system 6000 includes both implantable and external sensors, which can collectively be used to improve functional sensing and to improve energy efficiency of the VNS system 6000 as explained in more detail below.

The VNS system 6000 includes a VNS 6100, one or more first implantable sensors 6111A, one or more first external sensors 6211A, an electronic device 6200, a remote server 6400, and cloud-based infrastructure 6410. Each of the communication links shown in FIG. 6A between two components may independently be bidirectional or unidirectional in either direction.

The VNS 6100 may include an IPG 6110, two stimulation leads 6118 coupled to the IPG 6110, and two nerve cuffs 6124 respectively on the two stimulation leads 6118. The IPG 6110 may include implantable stimulation circuitry 6106 (e.g., an implantable stimulator, such as a pulse stimulator), an implantable controller 6120, and a second implantable sensor 6111B integrated with the VNS 6100. The one or more first implantable sensors 6111A and the one or more second implantable sensors 6111B may collectively be referred to herein as implantable sensor 6111. Although not shown, the VNS 6100 may also include a first transceiver configured to wirelessly communicate (e.g., wirelessly receive and/or transmit RF or Bluetooth signals) to other components of the VNS system 6000, such as the electronic device 6200, one or more of the first implantable sensors 6111A, and/or one or more of the first external sensors 6211A. The VNS 6100 may also include a memory storing instructions configured to cause the implantable controller 6120 to perform operations as described herein in response to executing the instructions stored in the memory. Wireless communication may be enabled by a Bluetooth, near-field communication (“NFC”), or other wireless communication protocol using a wireless modem integrated into the VNS 6100. In this example, the wireless modem is envisioned as a component of the implantable controller 6120 integrated into the housing 6102 of the VNS 6100.

The electronic device 6200 may be in the form of a wearable device 6200A, a stationary device 6200B, or a portable device 6200C, and may include an external controller 6220, a memory 6214, a transceiver 6216, and a second external sensor 6211B integrated with the electronic device 6200. In some examples, the electronic device 6200 may include a patient remote or a clinician programmer. The wearable device 6200A is depicted as having a smart watch form factor, but the wearable device 6200A may have any other form as described herein. In some examples, the wearable device 6200A may be configured to connect to the cloud 6410 via a Bluetooth low energy (BLE) connection. The one or more first external sensors 6211A and the one or more second external sensors 6211B may collectively be referred to herein as the external sensors 6211. The external sensors 6211 may be configured to connect with one or more of the electronic devices 6200 (e.g., a patient remote and/or external controller) via a BLE connection. The memory 6214 may store instructions configured to cause the external memory 6220 to perform operations as disclosed herein in response to executing the instructions. The transceiver 6216 is configured to allow the electronic device 6200 wirelessly communicate (e.g., receive and/or transmit signals) with other components of the VNS system 6000. In some examples, the transceiver 6216 is configured for both short range and long range communication. For example, the transceiver 6216 may be configured to wirelessly communicate (e.g., receive and/or transmit) RF or Bluetooth signals for communicating with the VNS 6100, the one or more first implantable sensors 6111A, and/or the one or more first external sensors 6211A, which may be within short range of the wearable device 6200. The transceiver 6216 may also be configured for cellular and/or Wi-Fi communication so that the transceiver 6216 can wirelessly communicate (e.g., receive and/or transmit signals) with the cloud-based infrastructure 6410 and/or the server 6400. In some examples, the cloud-based infrastructure 6410 may be configured to function as a communication intermediary between the electronic device 6200 and the server 6400. For example, the cloud-based infrastructure 6410 may provide a communication link to the remote server 6400. In some examples, the electronic device 6200 may be configured to communicate directly with the server 6400 (e.g., without using the cloud-based infrastructure as a communication intermediary). The one or more first external sensors 6211A may be separated from the electronic device 6200 and, in some examples, may be on separate wearable devices.

Each of the one or more first implantable sensors 6111A and one or more first external sensors 6211A may be configured to wirelessly transmit (e.g., via RF and/or Bluetooth signals) the sensed data to the VNS 6100 and/or to the electronic device 6200. Each of the sensors of the VNS system 6000 may independently be configured to sense at least one of heart rate, heart rate variability (HRV), respiration (e.g., respiration rate), patient movement (e.g., patient activity and/or mobility), patient position (e.g., upright or horizontal position), blood pressure, or oxygen saturation (SpO2). In some examples, any of the sensors of the VNS system 6000 may include an inertial measurement unit (IMU), an accelerometer, a gyroscope, an electroencephalogram (EEG) sensor, an electrocardiogram (EKG) sensor, an electromyography (EMG) sensor, a blood pressure sensor, and/or a pulse oximetry sensor. For example, one or more of the first and second implantable sensors 6111A and 6111B may include the IMU and/or the accelerometer, which may be configured to measure at least one of heart rate, HRV, respiration rate, patient movement, and/or patient position.

FIG. 6B is a schematic view of a first example of the VNS system 6000 of FIG. 6A according to some examples. The VNS system 6000 of FIG. 6B includes the VNS 6100 and the electronic device 6200 (in this example, a wearable device 6200A having a smart watch form factor) used for biometric sensing (e.g., having one or more integrated implantable sensors) and for controlling the VNS 6100. The electronic device 6200 may include a Bluetooth radio configured to connect to the VNS 6100 and that is configured to provide a simple user interface for a user to control stimulation parameters of the VNS 6100. The electronic device 6200 may be configured to function as a patient remote and for biometric sensing (e.g., heart rate, HRV, respiration rate, ECG, SpO₂, blood pressure, etc.). The electronic device 6200 may also have cellular and/or wi-fi connectivity to communicate with a server (e.g., via the cloud-based infrastructure 6410) for data sync and/or remote programming in a cloud-based environment. For example, data collected from (e.g., biometric data measured by) the electronic device 6200 and/or the VNS 6100 may be uploaded to a cloud server for remote data processing. Data can be uploaded using cellular or wi-fi connectivity. In certain conditions, specific events (e.g., value(s) of heart rate, HRV, respiration rate, and/or SpO₂) that were calculated by the server based on sensed data received by the server could be transmitted to the VNS 6100 and the VNS 6100 can change the stimulation accordingly.

FIG. 6C is a schematic view of a second example of the VNS system 6000 of FIG. 6A according to some other examples. The VNS system 6000 of FIG. 6C includes a first electronic device 6200 (in this case, a ring or other wearable device 6200A), which is used for biometric sensing (e.g., includes a heart rate sensor, HRV sensor, respiration rate sensor, oxygen sensor, blood pressure sensor, etc.), and a second electronic device 6300A (in this case, a handheld patient remote 6300A), which is used as a primary controller of the stimulation system. It should be understood that any of the systems described herein may incorporate a plurality of electronic devices 6200 (e.g., one or more wearable devices 6200A, one or more stationary devices 6200B, and/or one or more portable devices 6200C). Control and sensing functionality may be divided among the plurality of electronic devices 6200 in any manner (e.g., one electronic device 6200 may serve as a primary controller, with the others as backup or secondary controllers).

Referring collectively to FIGS. 6A-6C, the electronic device 6200 may be configured to connect to a remote server 6400 in order to obtain or transmit information (e.g., biometric parameters of the subject, or stimulation parameters to be used by the stimulation system). For example, the server 6400 may be operated by a medical professional (e.g., a clinician programmer) responsible for medical treatment of the subject. The medical professional may use the remote server 6400 to view and/or adjust one or more stimulation parameters (e.g., they may set or adjust a pulse frequency, pulse width, pulse amplitude, and/or duty cycle of the electrical stimulation) or set conditions for when stimulation should be applied (e.g., one or more biometric parameter thresholds that trigger a therapeutic intervention, such as the activation of stimulation or an increase/decrease in stimulation).

In some examples, the electronic device 6200 may be configured to upload historical data to the remote server 6400, such as biometric data sensed by any one or more of the sensors of the VNS system 6000 (e.g., historical biometric parameter levels of the patient) and/or recorded medical events (e.g., onset and/or duration of tachycardia, bradycardia, or a seizure). Such data may be used by a medical professional to monitor and/or evaluate the effect of stimulation parameters, so that therapy may be personalized for the subject. In some examples, the server 6400 may process (e.g., analyze) the data and/or calculate stimulation parameter(s) based on the data (e.g., the sensed biometric data) and transmit the stimulation parameters to the electronic device 6200, which can in turn transmit the stimulation parameter(s) to the VNS 6100 for implementation.

In some examples, the external controller 6220 is capable of setting or modifying one or more parameters of the VNS 6100. It is envisioned that this functionality may allow the electronic device 6200 to act as a master (primary) controller of the VNS 6100 when available (e.g., when communicatively linked with the VNS 6100), and the implantable controller 6120 can serve as a secondary or backup controller to maintain operation (e.g., when the electronic device 6200 is not in proximity or otherwise unavailable to the subject or inaccessible to the VNS 6100).

The electronic device 6200 (e.g., the external controller 6220) and the implantable controller 6120 may each be in communication with one or more of the implantable sensors 6111 and/or external sensors 6211. In some examples, both devices may be configured to maintain separate communication connections to the one or more sensors. However, energy may be conserved for the VNS system 6000 and for individual components (e.g., for the VNS 6100) by reducing the number of connections. For example, when the electronic device 6200 is available (e.g., detected based on proximity or by the establishment of a communications link with the implantable controller 6120), the implantable controller 6120 may disable communication with one or more (e.g., all) of the implantable sensors 6111 and/or one or more (e.g., all) of the external sensors 6211 in order to conserve the battery of the VNS 6100, and the electronic device 6200 can then take over collection of sensor data. In some such cases, the implantable controller 6120 may cease, and the external controller 6220 (or the server 6400) may take over processing (e.g., analyzing) the sensed data and/or calculating one or more stimulation parameters based on the sensed data. The electronic device 6200 may then establish a connection (or maintain an existing connection) with one or more of the implantable sensors 6111 and/or one or more of the external sensors 6211 that were previously linked with the implantable controller 6120.

In some examples, the electronic device 6200 may use additional or alternative sensors as a replacement for one or more sensors disabled by the implantable controller 6120. For example, the implantable controller 6120 may disable an internal IMU used to detect the subject's heart rate, HRV, movement data, bodily position data, and/or respiration rate, and the electronic device 6200 may obtain functionally equivalent data from an alternative external sensor 6211. The electronic device 6200 may use the sensor data collected from one or more of the implantable sensors 6111 and/or one or more of the external sensors 6211 to determine (e.g., calculate) one or more biometric parameters of the subject, and to determine whether stimulation is needed or if any stimulation parameters should be adjusted (e.g., based on the one or more determined biometric parameters). When it is determined that stimulation is needed (or if stimulation parameters should be adjusted), the electronic device 6200 may communicate any such instructions or commands to the implantable controller 6120 in order to trigger (or adjust) stimulation according to the selected parameters.

The electronic device 6200 may have a graphical user interface (“GUI”) (e.g., a display screen, such as a touch display) allowing a subject to view and/or modify one or more parameters of the VNS 6100, or to access other information about the VNS 6100. For example, the electronic device 6200 may provide a GUI that allows a subject to turn off or turn on stimulation, to set a schedule for the system. The GUI may also allow the user to immediately disable stimulation.

In some examples, the implantable controller 6120 may be configured to switch to a low power mode when the electronic device 6200 is available. In this mode, the implantable controller 6120 may disable one or more of the implantable sensors 6111 and external sensors 6211, and/or may reduce a polling rate or frequency of communication with one or more of the implantable sensors 6111 and external sensors 6211. The system may allow a user to manually activate this low power mode (e.g., using a GUI of the electronic device 6200). In some examples, the implantable controller 6120 may be switched back to a fully active mode (or “high power mode”) based upon a present condition (e.g., the termination of a wireless link with the electronic device 6200, a manual command input by the subject for example input via the GUI, or inability to detect the electronic device 6200 within a given amount of time). In the fully active mode, the implantable controller 6120 may exercise (e.g., resume or retake) control over stimulation (e.g., setting one or more stimulation parameters) and may optionally cease stimulation or disregard any previous stimulation parameters (e.g., as directed from the electronic device 6200).

In some examples, the implantable controller 6120 may be configured to poll all available sensors, or a predetermined set of the sensors, upon entering fully active mode. For example, the implantable controller 6120 may be configured to terminate all previous stimulation parameters upon entry into fully active mode, to poll all available sensors, and to proceed to determine one or more biometric parameters of the subject and whether intervention is needed. As such, the implantable controller 6120 may function as a backup controller capable of taking over the sensor monitoring and stimulation control features provided by the electronic device 6200 under set circumstances. This backup functionality may be useful, e.g., in cases where the electronic device 6200 becomes inoperable or inaccessible to the VNS 6100, for example, due to battery depletion, malfunction, or some other cause.

In some examples, the stimulation system may utilize a portable device 6200c (e.g., a handheld smart phone or dedicated control unit) configured to communicate with and control the VNS 6100. In some examples, the patient's biometric data is collected using sensors integrated into or communicatively linked to the portable device 6200c, or to a different electronic device 6200, such as a wearable device 6200A (e.g., a smart watch) or a stationary device 6200B (e.g., a bedside unit) configured to communicate with the portable device 6200C. As noted above, the control and sensing functions described herein may be performed by any electronic device 6200 described herein, or by a combination of such devices (e.g., where each device performs some or all of the control and/or sensing functionality). In some examples, the VNS system 6000 may include a plurality of electronic devices 6200, each assigned with a priority rank (e.g., a fixed rank), where the control and/or sensing functionality is performed by the highest ranking electronic device 6200 detected in proximity to the subject or the implantable controller 6120 (or based on any other criterion). For example, the VNS system 600 may prioritize use of a wearable device 6200A when a stationary device 6200B is not detected by the VNS 6100 or where a wireless signal emitted by the stationary device 6200B and received by the VNS 6100 is below a set threshold (e.g., a set quality threshold and/or a set strength threshold), and to switch control to the stationary device 6200B when that unit is detected or where the wireless signal emitted by the stationary device 6200B is above the set threshold. The same handoff process may occur with use of a portable device 6200C. In some examples, control may be assumed by the electronic device 6200 that has the highest battery level, greatest processing capabilities, strongest wireless signal, or any other rule. Alternatively, the patient remote or clinician programmer may manually set or select an electronic device 6200 as the primary controller (or set any other prioritization rules, ranks, and/or assignments).

External sensing devices such as smartwatches often detect heart rate using PPG (photoplethysmography). This signal is typically less sensitive to patient motion than is an implanted IMU, because the IMU senses motion by design. Therefore, using an external PPG will provide a less motion-sensitive detection of heart rate and respiration, which can be recovered from the heart rate signal. It is also more convenient to remove a smartwatch and recharge a smartwatch than to use an implant charger to recharge one's implantable device.

External devices also have some drawbacks. They are not implanted and thus are not guaranteed to be worn at all times. It is commonplace to recharge smart watches and other wearable devices at night, especially for some individuals that cannot tolerate wearing jewelry, watches, or other devices at night. Thus, it is convenient to use an IMU to detect the required physiological parameters when the patient sleeps, bathes, or is not wearing the external device for any other reason.

In some examples, whenever a suitable external accessory is available and connected to the VNS 6100, the VNS 6100 may be configured to cease its sensing capabilities once sensing data is available via the external accessory. For example, when heart rate monitoring data is available via a smart watch, then the IPG may stop utilizing its IMU to sense heart rate and/or heart rate variability.

In some examples, the VNS system 6000 may include an implantable alpha sensor (e.g., any one of the implantable sensors 6111) and an external beta sensor (e.g., any one of the external sensors 6211). The implantable alpha sensor 6111 may be configured to sense (e.g., measure) first biometric data (e.g., heart rate, HRV, respiration rate, patient movement, and/or patient position), and the external beta sensor 6211 may be configured to sense (e.g., measure) second biometric data (e.g., heart rate, HRV, respiration rate, patient movement, and/or patient position). In some examples, the first biometric data and the second biometric data may be the same. For example, the first and second biometric data may both include, or be used to obtain, heart rate data.

The implantable alpha sensor 6111 and the external beta sensor 6211 may both be configured to transmit their respective sensed data to a primary controller of the VNS system 6000, such as to the implantable controller 6120 or to the external controller 6220. The primary controller may be configured to control the electrical stimulation provided by the VNS 6100 (or other IMD configured to provide electrical stimulation), for example, by controllably initiating or terminating stimulation, selecting a stimulation mode or stimulation algorithm, and/or setting one or more stimulation parameters (e.g., pulse width, pulse frequency, pulse amplitude, and/or duty cycle of stimulation pulses). In some examples where the primary controller is not the implantable controller 6120 (e.g., the primary controller is the external controller 6220), controlling the electrical stimulation may include transmitting stimulation instructions to the VNS 6100, which may then be implemented by the VNS 6100 (e.g., by the implantable controller 6120) to provide the electrical stimulation based on the stimulation instructions. For example, the stimulation instructions may include instructions to initiate or terminate stimulation, to provide the stimulation according to a selected mode or algorithm, and/or to provide the stimulation with one or more selected stimulation parameters (e.g., pulse amplitude, pulse width, pulse frequency, and/or duty cycle), and the implantable controller 6120 may provide the stimulation according to the stimulation instructions (e.g., without changing the stimulation instructions).

The primary controller may be configured to control the electrical stimulation based on one or both of the first biometric data and/or the second biometric data. For example, the first and second biometric data may each include heart rate, HRV, and/or respiration rate, and the primary controller may be configured to control the electrical stimulation based on heart rate, HRV, and/or respiration rate. This may entail, for example, analyzing sensed heart rate, HRV, and/or respiration rate to detect the occurrence or onset of a seizure and, in response, initiating or increasing stimulation (e.g., increasing pulse amplitude) to counteract the seizure. In other examples, the primary controller may analyze the biometric data (e.g., present and/or historical biometric data) to determine whether to adjust stimulation treatment for epilepsy, and/or depression and, in response, to adjust one or more parameters of the electrical stimulation.

In some examples, the primary controller may be configured to selectively operate in an internal sensor mode or in an external sensor mode. For example, the primary controller may be configured to switch between at least the internal sensor mode and the external sensor mode based on whether certain threshold criterion is satisfied. In the internal sensor mode, the primary controller is configured to control the electrical stimulation based on the first biometric data sensed by the implantable alpha sensor 6111 (e.g., based only on the first biometric data or without the second biometric data). In the external sensor mode, the primary controller is configured to control the electrical stimulation based on the second biometric data sensed by the external beta sensor 6211 (e.g., based only on the second biometric data or without the first biometric data). In some examples, the primary controller is configured to not operate the implantable alpha sensor 6111 and/or to stop communicating with the implantable alpha sensor 6111 (e.g., terminate or suspend a communication link with the implantable alpha sensor 6111) when the primary controller operates in the external sensor mode.

The primary controller may be configured to selectively operate in the internal sensor mode or the external sensor mode based on at least one of a communication connection between the primary controller and the external beta sensor 6211, a distance between the primary controller and the external beta sensor 6211, a signal quality of one or both of the first and second biometric data, a time schedule (e.g., a scheduled day and/or intraday time period), a rank of one or both of the implantable alpha sensor 6111 and the external beta sensor 6211, a battery level of one or both of the implantable alpha sensor 6111 and the external beta sensor 6211, a processing capability value of one or both of the implantable alpha sensor 6111 and the external beta sensor 6211, or a user control signal (e.g., a signal received from the electronic device 6200, the patient remote, or the clinician programmer). The rank of a sensor may include an indication of how the sensor (e.g., use of the sensor) should be prioritized relative to other sensors of the system. For example, the ranks of multiple sensors in the system may be manually selected and/or based on one or more metrics (e.g., signal quality, processing capability, etc.) of the sensors. In some examples, the rank of a sensor may be stored in a memory in the system (e.g., a memory of the sensor device, of the external device 6200, of the VNS 6100, or of the server 6400).

For example, the primary controller may be configured to operate the VNS 6100 in the internal sensor mode in response to determining that a first threshold criterion is satisfied, and to operate the VNS 6100 in the external sensor mode in response to determining that a second threshold criterion is satisfied. In some examples, the first and second threshold criterion are mutually exclusive (i.e., have no overlap).

In some examples, the first threshold criterion includes at least one of the primary controller being communicatively disconnected from the external beta sensor 6211 (or with the electronic device 6200) for a threshold time period, a distance between the external beta sensor 6211 and an implantable component of the VNS system 6000 (e.g., the implantable controller 6120 or the VNS 6100) being greater than a threshold distance, a signal quality of the second biometric data being below a threshold value (e.g., being below a signal quality of the first biometric data), a current day and/or time (e.g., as measured by a clock of the VNS system 6000, such as a clock within the VNS 6100) being within a set schedule (e.g., a schedule stored in a memory coupled to the primary controller) for operating the VNS 6100 in the internal sensor mode, a rank of the implantable alpha sensor 6111 being higher than a rank of the external beta sensor 6211, a battery level of the external beta sensor 6211 (e.g., a battery level of the electronic device that the external sensor is part of) being below a threshold level (e.g., being below a battery level of the implantable alpha sensor 6111, such as of the VNS 6100), a processing capability value of the external beta sensor 6211 being below a threshold value (e.g., being below a processing capability value of the implantable alpha sensor 6111), or receiving a user control signal including instructions to operate the VNS 6100 in the internal sensor mode.

In some examples, the second criterion for operating in the external sensor mode includes at least one of the primary controller being communicatively connected from the external beta sensor 6211 for a threshold time period, a distance between the external beta sensor 6211 and an implantable component (e.g., the implantable controller 6120 or the VNS 6100) being less than a threshold distance, a signal quality of the second biometric data being above a threshold value (e.g., being above a signal quality of the first biometric data), a current day and/or time being within a set schedule (e.g., a schedule stored in a memory coupled to the primary controller) for operating the VNS 6100 in the external sensor mode, a rank of the external beta sensor 6211 being higher than a rank of the internal alpha sensor 6111, a battery level of the external beta sensor 6211 being above a threshold level (e.g., being above a battery level of the implantable alpha sensor 6111), a processing capability value of the external beta sensor 6211 being above a threshold value (e.g., being above a processing capability value of the implantable alpha sensor 6111), or receiving a user control signal including instructions to operate the VNS 6100 in the external sensor mode.

In some examples, the primary controller may also selectively operate in a multi-sensor mode, whereby the primary controller controls the electrical stimulation based on both the first and second biometric data. For example, the primary controller may be configured to selectively operate in the internal sensor mode, the external sensor mode, or the multi-sensor mode. The multi-sensor mode may allow the primary controller to control the electrical stimulation based on more accurate or reliable biometric data by relying on multiple biometric data obtained from multiple sensors. For example, if the first and second biometric data are both heart rate values, the primary controller may be able to rely on a more accurate heart rate value by using (e.g., integrating) both of the first and second heart rate values (e.g., by averaging the two values or applying a different weighting scheme based on, for example, the ranking or reliability of the alpha and beta sensors). In some other examples, the first and second biometric data may be different types of biometric data, such as heart rate and respiration rate, and the combination of biometric data may enable the primary controller to more accurately determine the patient's physiological condition (e.g., whether the patient is experiencing a seizure or the onset of a seizure).

The primary controller may be configured to operate in the multi-sensor mode in response to a third criterion being satisfied. For example, the primary controller may be configured to operate in the multi-sensor mode only if the primary controller has a communication link with the external beta sensor 6211 and/or a distance between an implantable component (e.g., the VNS 6100) and the external beta sensor 6211 is less than a threshold distance. More generally, the third criterion may be similar to, or the same as, the second threshold criterion described herein. In some examples, the primary controller may be configured to selectively operate in the internal sensor mode or in the multi-sensor mode (e.g., the primary controller may not be configured to operate in the external sensor mode).

FIG. 7 is a flowchart of a method 7000 relating to a processing workflow for determining whether an external sensor should be selected to replace an implantable sensor. For example, FIG. 7 may be used by the primary controller discussed above with reference to FIGS. 6A-6C to determine whether to use the external beta sensor 6211 or the implantable alpha sensor 6111 for controlling the electrical stimulation of the VNS 6100. During a first operation 7001, the method may include collecting biometric data using one or more implantable sensors (e.g., the implantable alpha sensor 6111). During a second operation 7002, the primary controller may monitor wireless communication channels to detect external sensors. When one is detected (e.g., when the external beta sensor 6211 is detected) during a third operation 7003, the system may establish a communication link with the external sensor during a fourth operation 7004. If, at the third operation 7003, an external sensor is not available, the primary controller may continue to monitor during the second operation 7002. After the wireless communication link is established during the fourth operation 7004, the primary controller may be configured to determine whether the external sensor should be used during a fifth operation 7005. This determination may be made based on a signal quality parameter and/or a logic regarding the selection of sensors. For example, the primary controller may be configured to select and use sensors having a signal quality above a set threshold, may be configured to select only certain types of sensors, and/or may be configured to select a minimum or maximum number of sensors of a certain type or class. The primary controller may also choose sensors based on manual user input, input from a clinician programmer (e.g., provided by a remote server 6400 operated by a clinician programmer), a schedule (e.g., one or more sensors may be selected for use at different times of day). In some examples, the primary controller may determine whether a sensor should be used by comparing the accuracy of its signal compared to sensor data collected from one or more other sensors (e.g., a comparison may show that sensor data from the new sensor is out of sync with data from a plurality of other sensors associated with the same biometric parameter).

If it is determined at the fifth operation 7005 that the external sensor should be used, the system may then utilize that external sensor to collect sensor data (e.g., biometric data) at a sixth operation 7006. Then, at a seventh stage 7007, the primary controller may be configured to determine whether the new external sensor can function as a replacement for an implantable sensor that is currently in use. For example, the system may have access to multiple sensors capable of obtaining data indicative of the heart rate, HRV, and/or respiratory rate of a subject. In this situation, multiple sensors may be used simultaneously (e.g., to improve accuracy and/or to detect when any individual sensor falls out of sync due to movement or other artifacts), or a single or reduced subset of sensors may be used (e.g., to conserve battery life). The primary controller may follow a set logic when making this determination. For example, the primary controller may be configured to select the external sensor as a replacement sensor for an implantable sensor if the external sensor is configured to provide a signal indicative of a biometric parameter currently being monitored by said implantable sensor and, in some examples, also if the external sensor provides energy efficiency, has a longer battery life, has a higher signal quality parameter, is identified as having higher accuracy than the current sensor, and/or has a higher rank than said implantable sensor. It is envisioned that any parameters disclosed herein may be used to determine whether an external sensor can replace use of an implantable sensor. If it is determined at the seventh operation 7007 that the external sensor can replace the implantable sensor, then the implantable sensor may be disabled at an eighth operation 7008. This may entail the implantable sensor ceasing to operate (e.g., ceasing to collect data) and/or the primary controller terminating a communication link with the implantable sensor.

When external accessories are not available or sensing data is not reliable, the IPG may be configured to turn its internal sensing ON (e.g., by utilizing or activating the implantable controller 6120 in order to collect sensor data from one or more of the implantable sensors 6111). For example, after the eighth operation 7008, if the primary controller loses its communication link with the external sensor, it may enable the implantable sensor that was disabled during the eighth operation 7008. For example, the primary controller may re-establish a communication link with the implantable sensor and/or cause the implantable sensor to continue sensing biometric data.

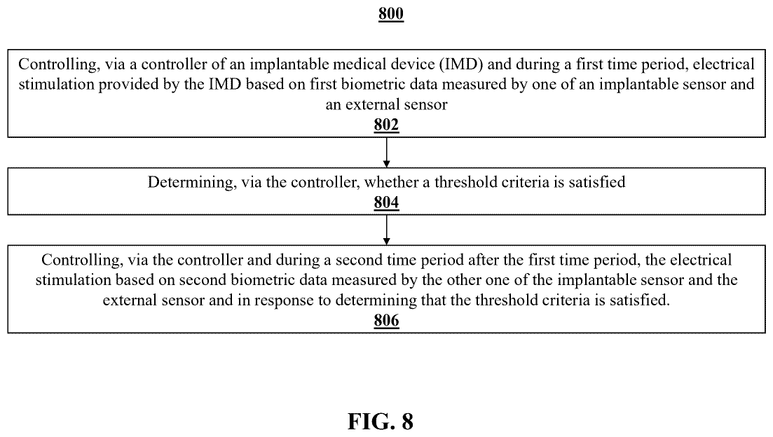

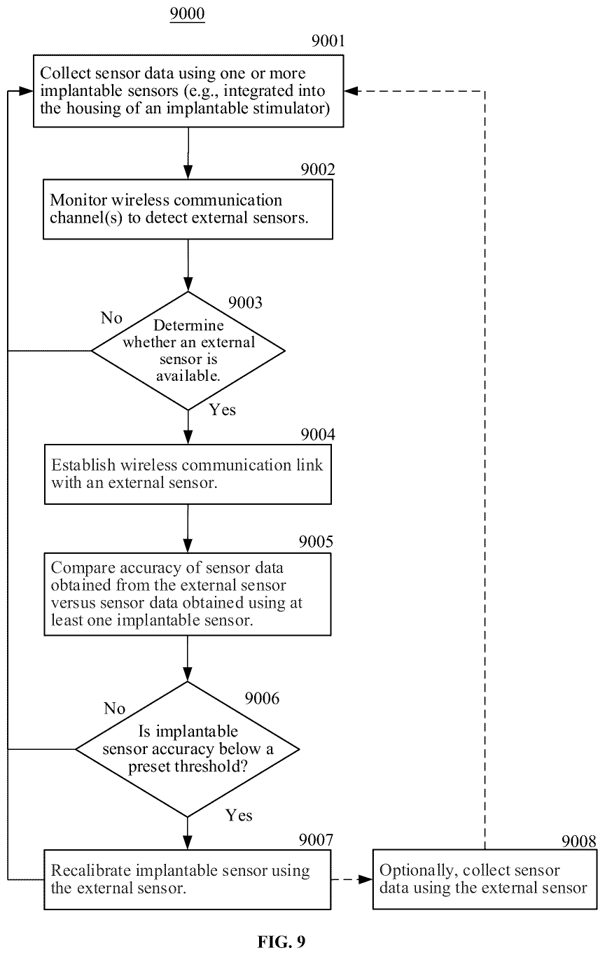

FIG. 8 is another flowchart diagram for another method 8000 of selectively using an external sensor or an implantable sensor for controlling electrical stimulation by an IMD. The method 8000 may be performed by a controller of the VNS systems 6000 of FIGS. 6A-6C, such as the primary controller.

The method 8000 may include a first operation 8002 of controlling, via a controller of a medical system and during a first time period, electrical stimulation provided by the IMD based on first biometric data measured by one selected from among an implantable sensor and an external sensor. For example, the IMD may be operating in the internal sensor mode or in the external sensor mode during the first time period. During a second operation 8004 (e.g., after the first operation 8002), the controller may determine whether a threshold criterion is satisfied. The threshold criterion may be, for example, the first threshold criterion discussed above for operating in the internal sensor mode or the second threshold criterion discussed above for operating in the external sensor mode. During a third operation 8006, based on the threshold criterion being satisfied, the controller may control the electrical stimulation based on the other one selected from among the implantable sensor and the external sensor. For example, in the third operation 8006, the controller may cause the IMD to transition between operating in the internal sensor mode and the external sensor mode.

A stimulator system, such as any of the VNS systems 6000 of FIGS. 6A-6C, may include a plurality of sensors that may be configured to measure the same biometric data (e.g., heart rate, HRV, respiration rate, etc.). Including multiple sensors may have various advantages. For example, as already mentioned above, relying on multiple sensors for the same type of biometric data can yield more accurate or reliable measurements for that type of biometric data. Another advantage is that, if any of these sensors experience measurement drift (i.e., become inaccurate or uncalibrated), the remaining sensors can be used as a reference point to recalibrate the drifting sensor. Thus, increasing the number of sensors in the stimulator system can improve the stimulator system's ability to monitor and recalibrate its own sensors over time.

A controller of the stimulator system, such as the implantable controller 6120, the external controller 6220, or a controller of the server 6400 may be configured to perform at least part of this recalibration process. For example, the controller may be configured to collect biometric data sensed by the plurality of sensors (e.g., one or more of the implantable sensors 6111 and/or one or more of the external sensors 6211) that are configured to measure the same type of biometric data. In some examples, the controller may perform this operation by polling some or all of the plurality of sensors, which may include establishing a communication link with at least some of these sensors that the controller does not have existing communication links with.

The controller may be configured to analyze the various biometric data from the plurality of sensors and determine if any of said biometric data is inaccurate (e.g., relative to the remaining biometric data). If the controller determines that a first sensor of the plurality of sensors is generating inaccurate data, it may determine that the first sensor has become uncalibrated and may recalibrate the first sensor, for example, based on the biometric data sensed by the remaining sensors of the plurality of sensors. The recalibration may be performed by any generally available processes for sensor calibration. In some examples, one of the implantable sensors 6111 may be recalibrated based at least in part on one or more of the external sensors 6211, or one of the external sensors 6211 may be recalibrated based at least in part on one or more of the implantable sensors 6111.

Having two devices (e.g., two sensors) to sense any physiological parameter provides the opportunity to compare the accuracy of the two sensors and notify the patient or clinician of a potential sensing error. If one sensor is susceptible to drift or other errors and needs periodic recalibration, the second sensor can provide a way to recalibrate. For example, implantable devices can rotate or flip. Using an external sensor that senses the beginning and end of respiration can be used to detect whether an implanted device or sensor has flipped, by signal inversion, or rotated, by change of the signal magnitude on each of multiple axes.

FIG. 9 is a flowchart of a method 9000 for recalibrating a sensor. In some examples, the method 9000 is performed by a controller of the VNS system 6000 described in FIGS. 6A-6C, and the sensor being recalibrated may be any one of the implantable sensors 6111 or external sensors 6211. For example, the method 9000 may be performed by the implantable controller 6120, the external controller 6220, or a controller of the server 6400.

The method 9000 may begin at a first operation 9001 with the collection (e.g., via a controller) of sensor data from one or more sensors (e.g., one of the implantable sensors 6111). At a second operation 9002, the system (e.g., the controller) may monitor wireless communication channels to detect whether one or more additional sensors are available (e.g., one or more of the external sensors 6211) at a third operation 9003. If, at the third operation 9003, it is determined that an external sensor is available, the controller may establish a wireless communication link with the external sensor at a fourth operation 9004. However, if at the third operation 9003 no external sensors are detected, the controller may revert to the first operation 9001. After establishing the wireless communication link with the external sensor at the fourth operation 9004, the system may then compare the accuracy of the sensor data obtained from the external sensor versus sensor data obtained using at least one other sensor (e.g., the implantable sensor) at a fifth operation 9005. If the accuracy of the implantable sensor is found to be less than the accuracy of the external sensor at a sixth operation 9006, then the system may proceed, during a seventh operation 9007, to recalibrate the implantable sensor using the external sensor as a baseline. In this case, the sixth operation 9006 includes using a threshold level of inaccuracy as the trigger for recalibration. During (or after) the recalibration of the seventh operation 9007, the system may proceed to utilize the external sensor as a replacement for the implantable sensor.

This figure illustrates one non-limiting example of a method for detecting sensor drift and performing a recalibration. Other methods may proceed using a different workflow and/or parameters. For example, a system may be configured to determine whether a sensor is inaccurate by periodically comparing the sensor data obtained from that sensor against a baseline sensor, or a plurality of sensors, capable of detecting a signal indicative of the same biometric parameter (or a different biometric parameter that can serve as a proxy for the biometric parameter at issue). A sensor may be found to be inaccurate when the determination of a biometric parameter based on that sensor's signal differs from the same determination made using sensor data obtained from the selected baseline sensor, or from an average or median value for a biometric parameter obtained using a plurality of sensors. As with the workflow shown in FIG. 9, a threshold may be used (e.g., a deviation of 1, 2, 3, 4, 5, 6, 7, 8, 9, 10, 15, or 20%, or a deviation within a range defined by any pair of integers between 1 and 100%).

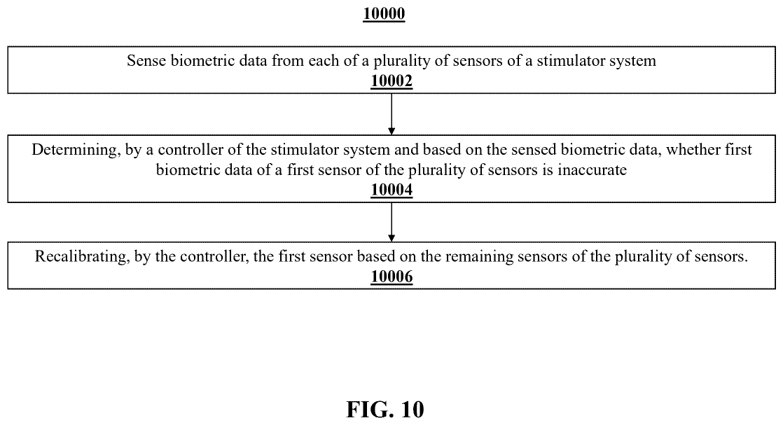

FIG. 10 is a flowchart for another method 10000 for recalibrating a sensor of a stimulator system. The stimulator system may be one of the VNS systems 6000 described with reference to FIGS. 6A-6C, the sensor for recalibration may be any one of the implantable sensors 6111 or of the external sensors 6211, and the method 10000 may be performed by a controller of the system, such as the implantable controller 6120, the external controller 6220, or a controller of the server 6400.

The method 10000 may include a first operation 10002 of sensing biometric data from each of a plurality of sensors of the stimulator system. For example, the plurality of sensors may include one or more of the implantable sensors 6111 and/or one or more of the external sensors 6211, such as a plurality of the implantable sensors 6111, a plurality of the external sensors 6211, or a combination of the implantable sensors 6111 and the external sensors 6211. The biometric data sensed by each of the plurality of sensors may all be of the same type, for example, heart rate, HRV, or respiration rate.

During a second operation 10004, a controller of the stimulator system may determine, based on the sensed biometric data of the plurality of sensors, whether any of the plurality of sensors are uncalibrated. This may be determined for a first sensor of the plurality of sensors by, for example, determining that first biometric data sensed by the first sensor is inaccurate (e.g., compared to the biometric data sensed by the remaining sensors of the plurality of sensors).

If it is determined that the first sensor is uncalibrated, the method 10000 may include a third operation 10006 of recalibrating the first sensor based on the remaining sensors, for example, based on the biometric data sensed by the remaining sensors. The recalibration may be performed by any generally known process for sensor recalibration.

In some examples, the VNS system 6000 of FIGS. 6A-6C, may be configured to selectively operate in an internal controller mode, whereby the implantable controller 6120 performs a set of one or more operations described herein, or in an external controller mode, whereby the external controller 6220 performs the set of operations. For example, the VNS system 6000 may be configured to controllably switch between operating in the internal controller mode and the external controller mode in response to determining, by one of the implantable controller 6120 or the external controller 6220, that threshold criterion has been satisfied.

The set of operations may be any combination of one or more operations from among all of the operations described herein that a controller may be configured to do. For example, the set of operations may include processing (e.g., analyzing) the biometric data sensed by the sensors of the VNS system 6000, controlling the electrical stimulation of the VNS 6100 (e.g., initiating or terminating stimulation, setting a stimulation mode or algorithm, and/or setting one or more stimulation parameters), recalibrating a sensor, generating and applying a personalized itcal model, titrating the electrical stimulation, and/or determining whether the patient has experienced a fall. Falls are often linked to the occurrence of a seizure, as they can result from a sudden loss of muscle control or consciousness during or after a seizure event. Therefore, identifying a fall can help in detecting seizure activity or monitoring seizure-related risks. The set of operations may include any combination of operations described with reference to any combination of the method figures and in any order. Shifting performance of some operations from the implantable controller 6120 to the external controller 6220 can improve energy efficiency of, and reduce the processing load on, the VNS 6100 by reducing the number of operations performed by the implantable controller 6120. This can improve functionality of the VNS 6100 and extend the battery life of the VNS 6100.

The VNS system 6000 may transition from operating in the internal controller mode to operating in the external controller mode in response to determining (e.g., by the implantable controller 6120 or the external controller 6220) that there is a communication link between the implantable controller 6120 and the external controller 6220. The VNS system 6000 may transition from operating in the external controller mode to the internal controller mode in response to determining (e.g., by the implantable controller 6120) that there has been no communication (e.g., no communication link) between the implantable controller 6120 and the external controller 6220 for a threshold time period.

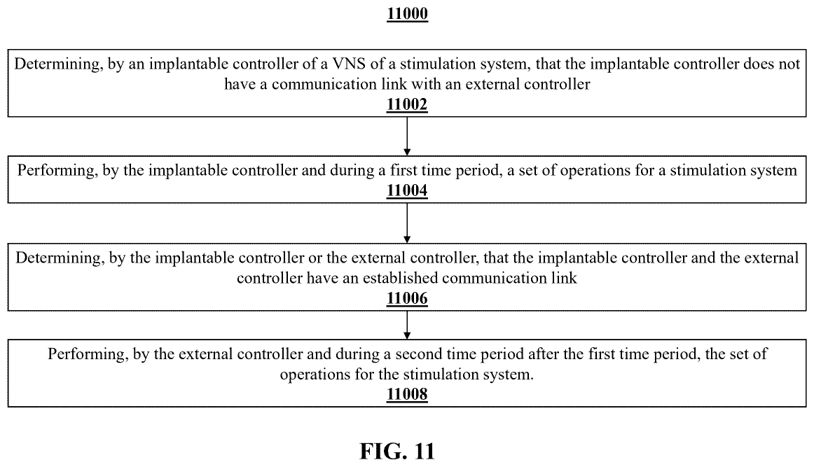

FIG. 11 is a flowchart diagram of a method 11000 for transitioning between the internal and external controller modes according to some examples. In some examples, the method 11000 may be performed by the VNS system 6000 of FIGS. 6A-6C.

The method 11000 includes a first step 1102 of determining, by an implantable controller of a VNS of a stimulation system, that the implantable controller does not have a communication link established with an external controller of the stimulation system. During a second operation 11004, in response to determining that there is no communication link, the implantable controller may perform a set of operations for the stimulation system during a first time period. The set of operations may be any combination of operations described herein. For example, the set of operations may include setting one or more stimulation parameters (e.g., pulse amplitude, pulse width, pulse frequency, and/or duty cycle) of the electrical stimulation provided by the VNS. In some examples, during the first time period, the external controller does not perform any of the set of operations.

The method 11000 may include a third operation 11006 of determining, by the implantable controller or the external controller, that the implantable controller and the external controller have an established communication link. This may occur in response to the communication link becoming established. During a fourth operation 11008, in response to determining that the communication link is established, the external controller may perform the set of operations during a second time period after the first time period. In some examples, during the second time period, the implantable controller does not perform any of the set of operations.

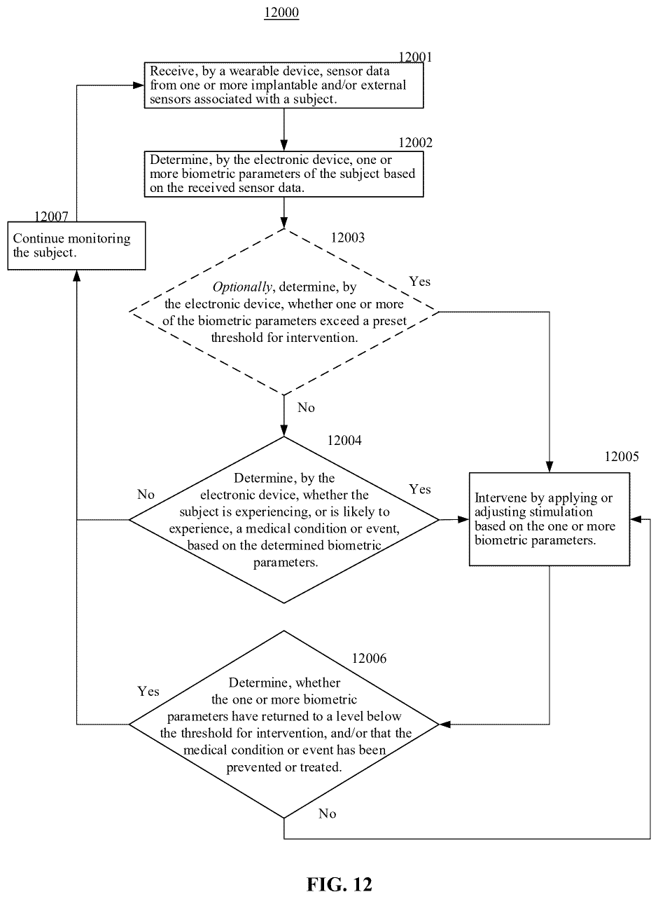

FIG. 12 is a flowchart diagram of a method 12000 for providing stimulation according to some examples. The method 12000 may be performed by a controller of one of the VNS systems 6000 of FIGS. 6A-6C, such as the implantable controller 6120, the external controller 6220, or a controller of the server 6400. In some examples, the method 12000 relates to a processing workflow for determining whether stimulation should be applied (or adjusted) as an intervention based on one or more biometric parameters of a subject (e.g., determined using data collected from one or more of the implantable sensors 6111 and external sensors 6211). The method 12000 may include a first operation 12001 of receiving, by the controller, sensor data from one or more of the implantable sensors 6111 and external sensors 6211. During a second operation 12002, the controller may process (e.g., analyze) this sensor data to determine one or more biometric parameters of the subject (e.g., heart rate, HRV, respiration rate, respiration cycle, the subject's temperature at one or more physiological locations, and/or the subject's position). The method 12000 may include an optional third operation 12003 of determining whether one or more of the determined biometric parameters exceeds a set threshold (e.g., falls below or rises above a normal or safe limit). If so, the processor may trigger intervention during a fifth operation 12005 by triggering stimulation (or adjusting one or more stimulation parameters, such as by increasing the pulse amplitude of pulse stimulation). If not, then the method 12000 may proceed to a fourth operation 12004. In some examples, the third operation 12003 is omitted, and the second operation 12002 instead proceeds directly to the fourth operation 12004.

The fourth operation 12004 may include determining, by the controller that a subject is experiencing, or is likely to experience, a medical condition or event, e.g., onset of seizure or precursor symptoms for seizure based on the determined biometric parameters. This determination may take into account one or more biometric parameters and optionally thresholds regarding the same, in addition to other data. For example, the determination may not be limited to biometric parameters at a given timepoint and may instead take into account historical data. For example, a determination that the subject is or experiencing a precursor physiological event before or at the onset of seizure may be based on the subject's respiratory rate and/or respiratory cycle, and also take into account the time of day, historical data obtained from the subject recorded during an seizure event, or other information. Upon determining that an intervention is appropriate, the system may then proceed to intervene at the fifth operation 12005 by triggering stimulation or adjustment of stimulation parameters. As illustrated by this figure, stimulation and monitoring may continue at a sixth operation 12006 until the subject's biometric parameters have returned to a normal level (e.g., determined using one or more thresholds), at which time the system may continue to monitor the patient at a seventh operation 12007 and return to the initial stage of the processing workflow.

### V. Seizure Detection in Closed-Loop Vagus Nerve Stimulation

Epilepsy is a group of neurological disorders characterized by recurrent seizures. During a seizure, or when a seizure is imminent, the patient may exhibit an increase in heart rate, referred to as ictal tachycardia, a decrease in respiration rate, which may be referred to as ictal apnea, and/or abrupt muscle contractions or spasms.

Seizures (e.g., clonic seizures) may be mitigated using vagus nerve stimulation, for example, as provided by any of the VNS systems described herein. Vagus nerve stimulation may involve applying an electrical stimulus to the vagus nerve. This stimulation may propagate to the brain and lessen the severity of a seizure, prevent a seizure, or stop a seizure that is in progress. For example, in an intervention referred to as open loop vagus nerve stimulation, the vagus nerve is periodically stimulated (e.g., for 5 seconds every 30 seconds) in an ongoing manner. In an intervention referred to as on-demand vagus nerve stimulation, the vagus nerve is stimulated in response to user input, for example, a button press or a screen click performed by the subject or by an assistant or a clinician. In an intervention referred to as closed-loop vagus nerve stimulation, the vagus nerve is stimulated automatically (e.g., without user intervention) in response to the detection, by a controller, that a seizure has started or is imminent.

Closed-loop vagus nerve stimulation may be performed by any VNS system described herein. FIG. 13A is a schematic illustration of a VNS 13100, which may be an example of, or have features similar to or the same as, the VNS 1100 of FIG. 1 or the VNS 6100 of FIGS. 6A-6C. Accordingly, redundant descriptions may not be repeated. The VNS 13100 may include an IPG 13110 having a housing 13100, a stimulation lead 13118 electrically coupled to the IPG 13110, and a nerve cuff (not shown in FIG. 13A) attached to a distal end of the stimulation lead 13118. The IPG 13110 may include (e.g., within the housing 13100) a battery 13112 (e.g., a rechargeable battery), an implantable controller 13120, stimulation circuitry 13106 (e.g., a VNS circuit) configured to provide electrical stimulation to the stimulation lead 13118 (e.g., under the control of the controller 13120, a first implantable sensor 13111B1, and a second implantable sensor 13111B2. In this example, the first implantable sensor 13111B1 includes a motion sensor, and the second implantable sensor 13111B2 includes a magnetometer.

In some embodiments, the first implantable sensor 13111B1 may be employed for the detection of seizures. The first implantable sensor 13111B1 may include, for example, at least one of an inertial measurement unit (IMU), accelerometer (e.g., a three-axis accelerometer, including three accelerometers, measuring acceleration along three different axes, such as along three orthogonal axes), or a gyroscope (e.g., a three-axis gyroscope, including three gyroscopes, measuring rotation rate along three different axes, such as along three orthogonal axes). As used herein, a “motion sensor” is a device that senses motion. As such, a single accelerometer is a motion sensor, and a set of three orthogonal accelerometers is also a type of motion sensor (and, as illustrated by this example, a motion sensor may include one or more motion sensors). An IMU can include an accelerometer, but it may additionally or alternatively incorporate other types of sensors. Some IMUs combine an accelerometer with a gyroscope to measure both linear acceleration and rotational motion.

The motion sensor may produce signals from which various biomarkers may be calculated. For example, the motion sensor may be used to generate so-called seismocardiograms or gyrocardiograms from which instantaneous heart rate (HR) may be calculated. The same sensor signals may be used to calculate the instantaneous respiration rate (RR) as well. A method for extracting the respiration rate may be to low-pass filter the inertial sensor signals, since motion due to breathing happens on a longer time scale than mechanical vibrations due to the heart. A neck mounted or implanted motion sensor may be used to measure pulsing of blood through the carotid artery. As used herein, a “biomarker” may refer to an indicator of one or more examples of the biological state or condition of the subject. As such, a biomarker may be the subject's heart rate and/or respiration rate.

Each of the accelerometers and gyroscopes may be a microelectromechanical systems (MEMS) sensor, e.g., fabricated on a semiconductor chip using photolithographic or analogous methods, and including, e.g., a cantilevered beam that may deflect when the first implantable sensor 13111B1 is subject to acceleration, or a resonant structure within which energy may couple between different resonant modes when the first implantable sensor 13111B1 is rotated.

The controller 13120 may include a processing circuit (discussed in further detail below) for receiving signals from or controlling the other circuits of the implantable device 13100. The second implantable sensor 13111B2 may be used, for example, to detect strong magnetic fields, the presence of which may indicate the likelihood that a seizure will occur. The blocks illustrated in FIG. 13A may be implemented in hardware or in some combination of hardware, software, and firmware. For example, each of the first implantable sensor 13111B1 and the second implantable sensor 13111B2 may include analog sensors and analog-to-digital converters.

FIG. 13B is a schematic view of some components of the IPG 13110 of FIG. 13A. FIG. 13B shows the first implantable sensor 13111B1 including an accelerometer 13150 (e.g., a three-axis accelerometer) and a gyroscope 13155 (e.g., a three-axis gyroscope), connected to a front end and digitizer circuit 13160 (which may include one or more analog preamplifiers and one or more analog to digital converters) and a signal processing circuit 13165 (which may be a processing circuit (discussed in further detail below)). A system like that shown in FIG. 1B, or a portion of such a system (e.g., the first implantable sensor 13111B1) may be used to obtain seismocardiograms or gyrocardiograms.

Although the first implantable sensor 13111B1 is primarily described herein as being part of the VNS 13100, in some other examples the motion sensor may be a separate implantable sensor (e.g., one of the first implantable sensors 6111A) or an external sensor (e.g., any one of the external sensors 6211), and may be configured to wirelessly communicate with the implantable controller 13120. Additionally, although the implantable controller 13120 is primarily described herein as being configured to perform various operations, such operations may alternatively or additionally be performed by one or more other controllers of the VNS system (e.g., the external controller 6220 and/or a controller of the server 6400). For example, the operations described herein may be performed by, or divided in any manner between any two or all three of, the implantable controller 13120, an external controller (e.g., the external controller 6220), and a controller of a remote server (e.g., the server 6400).

Automatic detection of seizures based on instantaneous heart rate calculations may be used to initiate stimulation in epileptic patients with implanted closed-loop, vagus nerve stimulation devices. In particular, hypothesis testing may be applied to sequential heart rate calculations to detect the presence of ictal tachycardia. Such detection strategies may involve computation of a test statistic and the comparison of the test statistic to some threshold.

Changes in instantaneous heart rate due to ictal tachycardia may be subject-specific, e.g., may vary in form from subject to subject and may be indicative of the onset of seizure or the occurrence of seizure. For example, the total increase in heart rate and/or the instantaneous rate of increase may be subject-specific. As such, some embodiments employ personalized, per-patient seizure detection, which may test for particular forms of heart rate increase characteristically associated with an individual patient. Testing of this type may lead to improved receiver operating characteristic (ROC) or lower latency. For example, the characteristics of multiple ictal-tachycardia-related instantaneous heart rate increases may be observed for each subject, and the observations may be used to create a subject-specific system or method for detecting that a seizure is imminent or occurring.

In some embodiments, the average heart rate history during ictal tachycardia is calculated (e.g., sensed by the first implantable sensor 13111B1 and/or by one or more other sensors of the VNS system configured to measure heart rate) for a patient, for example, by averaging together multiple heart rate histories (e.g., calculated heart rate histories) corresponding to multiple respective occurrences of ictal tachycardia. The current heart rate history (e.g., calculated heart rate history) of the subject may be compared, continuously or periodically, (i) to this average heart rate history during ictal tachycardia, and (ii) to a set of heart rate histories when ictal tachycardia is not occurring (or a statistical characterization of such a set) to determine whether a seizure is imminent or occurring. Such a comparison may include calculating a measure of similarity between the current heart rate history and the average heart rate history during ictal tachycardia. The calculation may be performed for example, by fitting the average heart rate history during ictal tachycardia with a parametric model to obtain a set of reference parameter values, fitting the current heart rate history of the subject with the parametric model to obtain a set of current parameter values, and comparing the current parameter values to the reference parameter values. As used herein, a heart rate history (or calculated heart rate history) is a sequence of calculations of the heart rate, extending over some interval of time (e.g., in the case of the current heart rate history, a time interval extending backward, from the present time, to a point in the past). The interval may have a length of between 5 seconds and 6000 seconds.

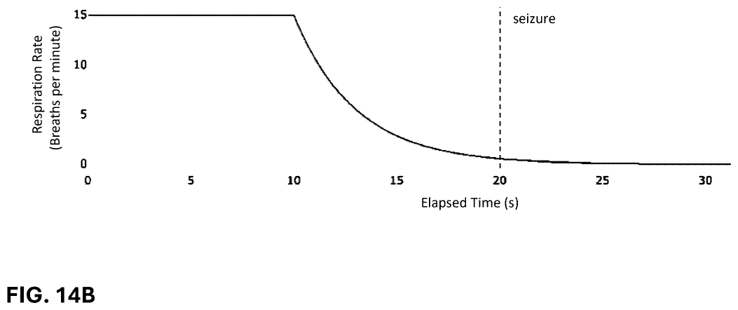

FIG. 14A shows a graph of one example of a parametrized model of ictal tachycardia; in this model the heart rate is modeled as following an exponential function, which is parametrized by a start time, an amplitude (of 25 beats per minute, in the example of FIG. 14A) (e.g., a change or increase in amplitude), and a time constant (of 6 seconds, in the example of FIG. 14A). For such a model, a modest increase in calculated heart rate (e.g., due to a slight increase in exertion in the subject, or due to noise in the first implantable sensor 13111B1) may result in the current heart rate history being best fit by a set of parameters in which the amplitude is small, or the time constant is large. As such, the system may continuously or periodically compare the amplitude and time constant corresponding to the current heart rate history to respective thresholds, and determine that a seizure is imminent or occurring if both (i) the amplitude exceeds the threshold amplitude and (ii) the time constant is less than the threshold time constant. In some embodiments, the ratio of the amplitude to the time constant (a ratio that may be proportional to the maximum rate of change, in the model) is compared to a threshold and the system determines that a seizure is imminent or occurring if the ratio exceeds a threshold.

FIG. 14B shows a graph of an exponential model of respiration rate that may be employed in an analogous manner to determine whether a measured change in the respiration rate is ictal apnea, for example, whether a seizure is imminent or occurring. In the model shown in FIG. 14B, the respiration rate is modeled as following an exponential function, which is parametrized by a start time, an amplitude (of 15 breaths per minute, in the example of FIG. 14B) (e.g., a change or decrease in amplitude), and a time constant (of 3 seconds, in the example of FIG. 14B). As in the example of FIG. 14A, the system may determine that a seizure is imminent or occurring if both (i) the amplitude, in the model, exceeds a threshold amplitude and (ii) the time constant, in the model, is less than the threshold time constant, or if the ratio of the amplitude to the time constant exceeds a threshold. In some embodiments, changes in heart rate and changes in respiration rate are jointly taken into account when a determination is made as to whether a seizure is imminent or occurring. In each of FIG. 14A and FIG. 14B, a dashed line shows a point in time at which a seizure may have begun.

In some embodiments, a different parametrized model of the heart rate is used. Such a model may be any function or representation that, when fit to a heart rate history, results in parameter values that may be used to determine whether a seizure is imminent or occurring (e.g., parameter values that depend on whether a seizure is imminent or occurring). For example, a parametrized model may be a polynomial model, a frequency-domain model, a wavelet domain model, or a matrix-based model such as empirical mode decomposition. In some embodiments, the system uses a model based on an average waveform that is an average of heart rate histories previously observed for the subject during a seizure. In such an embodiment, the parameters may be (or include) an amplitude (an overall scale factor by which the average waveform is multiplied) and a time offset.

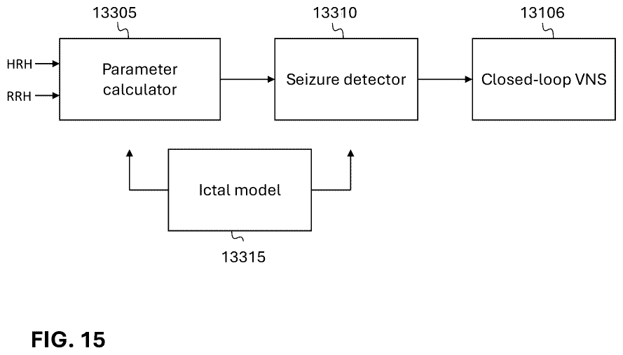

FIG. 15 is a block diagram of a system or method for determining whether a seizure is imminent or occurring and for performing closed-loop vagus nerve stimulation. A parameter calculator 13305 (which may also be referred to as a feature generator) is configured to receive a calculated heart rate history (HRH) and/or a calculated respiration rate history (RRH) and to fit a parametrized model to at least one of the one or more histories it receives, to generate a set of one or more parameter values. This may be repeated periodically, for example, once per second. The parameter values are fed to a seizure detector 13310, which may be configured to make a determination, for example, for each set of parameter values, of whether a seizure is imminent or occurring. Each of the parameter calculator 13305 and the seizure detector 13310 may be implemented in software (e.g., firmware running on the controller 13120) and/or in hardware (e.g., in a dedicated circuit, or using a general-purpose processing circuit separate from the controller 13120).

The seizure detector 13310 may make this determination based on any combination of the parameter values it receives. For example, the seizure detector 13310 may make the determination based on parameter values of the heart rate model, or based on parameter values of the respiration rate model (or on both). The heart rate model and/or the respiration rate model are schematically depicted in FIG. 15 as the ictal model 13315 block. In an embodiment in which each of the heart rate model and the respiration rate model is an exponential function (as discussed in the context of FIG. 14A and FIG. 14B), the seizure detector 13310 may for example, determine that a seizure is imminent or occurring (i) if the amplitude of the heart rate model exceeds a first threshold and the time constant of the heart rate model is less than a second threshold and/or (ii) if the amplitude of the respiration rate model exceeds a third threshold and the time constant of the respiration rate model is less than a fourth threshold. The thresholds may be determined based on (i) training (as discussed in further detail below), and otherwise fixed, or on (ii) other parameter values. For example the third and fourth thresholds may be determined based on the parameter values of the heart rate model (with, e.g., the third and fourth thresholds being lower and higher, respectively, if the ratio of the heart rate model amplitude to the heart rate model time constant is greater). When the seizure detector 13310 determines that a seizure is imminent or occurring, it may send a signal (e.g., a command) to the vagus nerve stimulation circuit 13106 to cause the vagus nerve stimulation circuit 13106 to perform vagus nerve stimulation.

The multiple sensors of the VNS 13100 of FIG. 13A may be used to identify scenarios associated with higher risk of seizure. For example, if it is found, for a particular subject, that seizures are more likely at a certain time of day (e.g., in the morning) or in the presence of a strong magnetic field, such information may be reported to the subject. Moreover, when such conditions are encountered, an elevated risk warning may be communicated to the subject, or a detection threshold may be modified.

In some embodiments, the seizure detector 13310 is or includes a machine-learning model, for example, a machine-learning classifier, such as an adaptive boosting classifier (AdaBoost), an artificial neural network (ANN) learning algorithm, a Bayesian belief network, a Bayesian classifier, a Bayesian neural network, a boosted tree, a case-based reasoning classifier, a classification tree, a convolutional neural network, a decisions tree, a deep learning classifier, an elastic net, a fully convolutional network (FCN), a genetic algorithm, a gradient boosting tree, a k-nearest neighbor classifier, a least absolute shrinkage and selection operator (LASSO) classifier, a linear classifier, a naive Bayes classifier, a neural network (e.g., a discriminative neural network), logistic regression (e.g., penalized logistic regression), a random forest, ridge regression, a support vector machine, or a combination thereof. Such a machine-learning classifier may be trained (e.g., using supervised training) as discussed in further detail below.

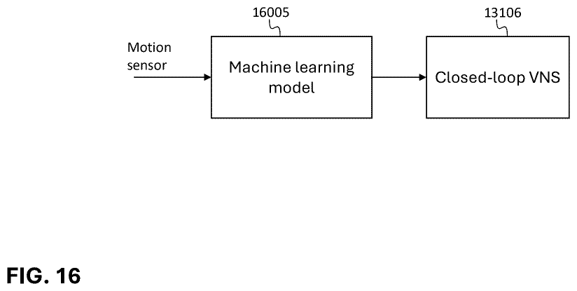

In some embodiments, the parameter calculator 13305 and the seizure detector 13310 are both machine-learning models. In such an embodiment the combination of the parameter calculator 13305 and the seizure detector 13310 may be considered to be a single (composite) machine-learning model, with an internal feature map that includes (e.g., consists of) the calculated heart rate of the subject. In some embodiments, a single machine-learning model performs the functions of the combination of the parameter calculator 13305 and the seizure detector 13310. An example of a machine learning model 16005 is schematically illustrated in FIG. 16. Such a machine-learning model need not have an internal boundary between a first portion that performs parameter calculation and a second portion that performs seizure detection, at which parameter values of a parametrized model for the calculated heart rate is transmitted from the first portion to the second portion; instead (i) latent variables that form the output of the first portion may be, for example, linear combinations of (or nonlinear functions of) the calculated heart rate and the calculated respiration rate, or (ii) (as illustrated in FIG. 16) the machine-learning model may map detected motion (e.g., the raw signals from motion sensors) directly to a determination of whether a seizure is imminent or occurring without using any internal (latent) variables that correspond directly to heart rate or respiration rate. In the latter embodiment (in which the machine-learning model may map detected motion directly to a determination of whether a seizure is imminent or occurring), or in other embodiments disclosed herein, machine learning algorithms that may offer attractive performance when trained on imbalanced data sets (e.g., data sets including far more data without seizure than with) and accept time series data (e.g., raw multi-dimensional time series data) include convolutional neural networks (e.g., fully convolutional networks) and recurrent neural networks (e.g., long short-term memory (LSTM) approaches). In any such embodiment the determination of whether a seizure is imminent or occurring may be used to perform closed-loop vagus nerve stimulation (as illustrated, for example, in FIGS. 15 and 16), with, for example, closed-loop vagus nerve stimulation being performed in response to a determination that a seizure is imminent or occurring.

Some methods described herein may be performed by the implantable device 13100 (e.g., by the controller 13120 of the implantable device 13100). In some embodiments, some methods (e.g., the training of one or more machine-learning models) may be performed by other computing systems (e.g., to reduce the computational burden on the controller 13120) as described herein. For example, the implantable device 13100 may be connected by a wireless connection to the electronic device 6200 of FIGS. 6A-6C (e.g., a mobile device, such as a mobile telephone, a laptop computer, or a tablet computer), which may perform some of the methods (e.g., the training of a machine-learning model) described herein, or which may relay data (e.g., training data and machine-learning model parameters, such as weights of a neural network) between the implantable device 13100 and another computing system, such as the server 6400, which may perform some of the methods (e.g., the training of a machine-learning model) described herein.

Training of the machine-learning models described herein may include supervised training, and may result in a subject-specific machine-learning model (e.g., a machine-learning subject-specific classifier). For example, during a training interval (which may be an interval with a length of between 5 days and 10 years, such as between 3 weeks and 50 weeks) the subject may report occurrences of seizures (e.g., when the subject senses that a seizure is imminent, or when the subject becomes aware that a seizure has occurred), or the system may detect seizures by the occurrence (as measured by the first implantable sensor 13111B1) of motions (e.g., shaking of the subject's torso in a way consistent with clonic seizure convulsions), that would result from muscle movements characteristic of a seizure. When a seizure is reported or detected, a record of motion sensor data (or a record of calculated heart rates and/or a record of calculated respiration rates) may be saved, and labeled as positive, e.g., as data corresponding to the occurrence of a seizure.

In some embodiments, such data records, each of which may be referred to as a “positive data record”, may be shifted in time (e.g., based on the time at which the seizure was reported and based on whether a recent seizure or an imminent seizure was reported) such that the start times of the seizures are approximately aligned in all of the positive data records. The positive data records may then be truncated at the beginning or at the end, such that all of the positive data records have approximately the same (e.g., within 50%, 20%, 10%, 5%, or 1% of being the same) length and all of the positive data records include data corresponding to a time interval during which a seizure began at approximately the same time within each interval. In other embodiments, each positive data record corresponds to a time interval during which a seizure occurred, and each positive data record may be labeled with the calculated time at which the seizure occurred in addition to being labeled as positive. In such an embodiment, the classifier may be trained to produce both (i) a determination of whether a seizure is imminent or occurring and (ii) a calculation of when the seizure began or will begin.

Training data corresponding to time intervals during which no seizure occurred, or “negative data records”, may be obtained by saving data for time intervals during which no seizures were reported by the subject, or during which motions that would result from muscle movements characteristic of a seizure are absent from the motion sensor data. Supervised training may be performed with the labeled training data (e.g., with the positive data records and with the negative data records), e.g., using back propagation, to produce learned parameters, or “weights” that may be stored for use by the machine-learning model. The training may be performed by the controller 13120, or a more capable computing system (e.g., a computing system connected directly to the implantable device 13100, or a server connected to the internet, or a plurality of servers connected to the internet) may be used to perform the training, e.g., to reduce the time required to complete the training. In some embodiments, the training is performed after a set of training data has been collected. In some embodiments, training is instead or is also performed during operation. For example, the system may begin operation with an initial set of machine-learning model weights; this initial set of weights may be ones obtained by training the machine-learning model with positive data records and negative data records obtained from a plurality of other subjects. Subject-specific training data may then be collected from the subject, while the system is operating (e.g., performing seizure detection and closed-loop vagus nerve stimulation), and the weights may be modified by, or replaced with new weights obtained from, further training using the subject-specific training data.

Each of the elements or methods for performing a data processing function (e.g., for performing heart rate calculation, for performing respiration rate calculation, for performing parameter calculation, or for performing seizure detection) described herein (e.g., shown in FIG. 15) may be implemented in hardware (e.g., as one or more circuits, e.g., processing circuits) or in software, or in a combination of hardware and software.

As used herein, a “subject-specific” system or method is a system or method the behavior of which is tailored to a subject, e.g., a system or method the behavior of which is based on one or more characteristics of the subject, the characteristics being ones that are different in another subject. As such, a subject-specific classifier may be a classifier that uses parameters (e.g., thresholds) based on characteristics that vary from subject to subject (e.g., a machine-learning model trained with subject-specific training data). For example, a subject-specific classifier may be trained with data obtained during seizures experienced by the subject. To the extent that such data includes unique characteristics not found in data from any other subject, the subject-specific classifier may be unique to a particular subject. In other embodiments, a subject-specific classifier may instead be tailored to a group of subjects, e.g., a group based on the age, ethnicity, height or weight of the subject. For example, if the heart rate or respiration rate profiles, during seizures, are typically different for subjects in different age ranges, then a subject-specific classifier may be one that is trained or otherwise configured to detect a heart rate or respiration rate profile expected during seizure for the age range within which the subject is found.

Although some examples discussed herein discuss the use of an accelerometer to generate a seismocardiogram, from which a heart rate may be calculated and used for seizure detection, the present disclosure is not limited to such embodiments. For example, a gyroscope may be used in an analogous manner to generate a gyrocardiogram from which a heart rate may be calculated, or a combination of a gyroscope and an accelerometer may be used to generate a signal that responds to cardiac motion, and that may be used, for example, to calculate the heart rate. Similarly, a respiration rate may be calculated from motion sensor signals (e.g., signals from one or more accelerometers or one or more gyroscopes) and used (by itself, or with the heart rate) for seizure detection.

The embodiments described herein may result in improved performance (e.g., improved receiver operating characteristics (ROC)) in detecting seizures and therefore improved performance in mitigating seizures using closed-loop vagus nerve stimulation. As such, these embodiments improve the technology of seizure detection and the technology of closed-loop vagus nerve stimulation.

### VI. Titration of Vagus Nerve Stimulation

Vagus nerve stimulation (VNS) may be used to treat one or more medical conditions, such as epilepsy and depression. However, VNS can cause undesirable side effects in addition to the anticipated therapeutic effect. These side effects may include laryngeal effects such as voice changes, sore throat, bradycardia and tachycardia. Bradycardia and tachycardia are of particular concern as they can lead to serious complications. Bradycardia refers to a condition in which the heart beats abnormally slow and can occur with higher levels of VNS stimulation. Tachycardia refers to a condition in which the heart beats abnormally high and can occur with lower levels of VNS stimulation. FIG. 17 depicts a graph of heart rate change (vertical axis) versus pulse amplitude (horizontal axis) of pulse stimulation that illustrates these affects. It can be seen that as the pulse amplitude of the stimulation increases from zero, the stimulation causes the patient's heart rate to increase above a normal level (tachycardia) to a peak (or hump) and then to decrease back to a neural fulcrum point where stimulation does not cause any change in heart rate. As the pulse amplitude continues to increase beyond the amplitude associated with the neural fulcrum, the heart rate continues to decrease below the normal level (bradycardia). To avoid the undesirable affects associated with tachycardia and bradycardia, it can be advantageous to provide stimulation at or around the neural fulcrum.

In current VNS systems, the stimulation amplitude is titrated slowly up from a minimum pulse amplitude with increases happening on the order of every week or every other week. A reason for this slow increase is to provide enough time to determine whether the current stimulation amplitude is having any beneficial effects and/or deleterious effects on the patient. However, the result of this overly conservative titration is that it can take 6 months or more to see a clinical effect.

As stimulation amplitude is increased, type A fibers in the vagus nerve will be recruited first. These include the motor neurons for the larynx. As stimulation amplitude increases further, type B fibers will be recruited. These include the afferent fibers that cause the intended therapeutic effect for certain maladies, such as epilepsy and depression, and efferent fibers that can cause undesirable side effects, such as tachycardia and bradycardia. Thus, limiting the stimulation below the level of any heart-rate effects may limit the overall effectiveness and/or delay the onset of benefit of the therapy.

The devices, systems, and methods for titration of VNS stimulation described herein address various shortcomings in the art, such as by utilizing algorithms and/or stimulation parameters based on sensor data collected from the subject in order to mitigate the issue of tachycardia and bradycardia associated with previous VNS-based therapeutic methods. In some examples, titration of VNS is performed at levels higher than typical for prior methods by increasing the stimulation pulse amplitude either to or near the neural fulcrum. As explained herein (and illustrated by FIG. 17), as VNS is increased, no heart rate change may be observed up to a certain amplitude. Above this amplitude, the heart rate will increase to a point and then begin to drop. The neural fulcrum is defined as that stimulation amplitude where the heart rate has dropped back to its original (normal) rate. In some examples, the best electrode or electrodes for administering stimulation may be selected based on laryngeal muscle activation and/or heart rate changes (e.g., as sensed by one or more sensors of the VNS system). For example, one or more electrodes of the VNS may be selected to reduce or minimize laryngeal muscle activation and/or cardiac/heart rate changes. Titration may be initiated, for example, at a pulse amplitude below the amplitude that causes laryngeal muscle contraction. As described in further detail herein, the high level of stimulation of the vagus nerve provided by the present systems and methods allows for effective treatment of multiple conditions while reducing, if not eliminating, the problems of tachycardia and bradycardia.

The present disclosure is based in part on findings that have revealed new insights into the heart rate side effect that patients undergoing VNS can suffer. As the level of VNS is increased above a certain threshold (typically different for each patient), the patient may experience an increase in heart rate (tachycardia). However, as the level of stimulation increases further, the heart rate typically levels out (e.g., peaks) and then starts to decline. The decline in heart rate will drop to the neural fulcrum (see FIG. 17) where there is no heart rate response to stimulation. Bradycardia will typically accelerate and be exacerbated as stimulation continues to increase, potentially becoming unhealthy, or even life threatening. Both tachycardia and bradycardia are undesirable conditions (e.g., can have deleterious effects on the patient's health) if they are more than a few beats per minute. However, it is desirable to maximize the stimulation of the vagus nerve in order to maximize the beneficial effects such as reduction in depression, epileptic seizure frequency, inflammation related maladies such as rheumatoid arthritis, sympathetic tone related issues such as excessive nervousness, auto-immune conditions, and others.

The present disclosure provides systems and methods of titrating VNS intensity either to or beyond the neural fulcrum “hump,” where the heart rate is increased above normal, to the point where the rate is on a downward trend and equal or close to the patient's normal heart rate. As described herein, a level of stimulation that elicits a normal heart rate response may be achievable using a closed-loop control system with heart rate measurement as an input. The heart rate may be measured by one or more of several methods such as electrocardiogram (ECG) measurement, inertial measurements, photoplethysmography, and others, during fitting sessions. The subject's heart rate may also be measured using one or more implanted and/or external sensors (e.g., a sensor integrated into the implanted stimulator).

In some examples, the systems described herein may further be configured to monitor and/or account for other factors, such as the subject's activity level. Activity level may be determined, e.g., using one or more sensors integrated into or communicatively linked with an implanted stimulator. External sensors may also be used, such as an inertial measurement unit (IMU), in order to detect and/or measure chest or diaphragm expansion or motion (by the subject generally, or of one or more anatomical locations). The closed-loop control algorithms described herein may also include safety thresholds for a minimum and maximum heart rate where the device may be programmed to cease stimulation once those limits are crossed.

In some examples, any of the VNS systems described herein, such as any of the VNS systems 1000 and 6000 of FIGS. 1 and 6A-6C, may be configured to perform the VNS titration processes described herein. For example, one or more controllers (e.g., the implantable controller 6120, the external controller 6220, and/or a controller of the server 6400) of the VNS system may be configured to perform the processes of the VNS titration described herein. The sensed data that the VNS titration may be based on data collected from one or more of the sensors of the VNS systems described herein (e.g., one or more of the implantable sensors 6111 and/or one or more of the external sensors 6211).

The VNS system 6000 may be configured to provide stimulation (e.g., pulse stimulation), to gradually (e.g., in a step-wise manner) increase the amplitude (e.g., pulse amplitude) of the stimulation, and to sense (e.g., measure) the patient's heart rate and/or detect for electromyography (EMG) (e.g., via one or more of the implantable sensors 6111 and/or one or more the external sensors 6211) as the amplitude is increased. For example, the VNS system 6000 may perform an iterative process of measuring the patient's heart rate before applying stimulation (referred to herein as a before heart rate HRb), then applying the stimulation with a set amplitude and measuring the patient's heart rate while applying the stimulation with the set amplitude (referred to herein as a during heart rate HRd), then stopping the stimulation and measuring the patient's heart rate after the stimulation has been stopped (referred to herein as an after heart rate HRa), and then increasing the set amplitude for the next iteration. In some examples, the before and after heart rates may be measured while providing stimulation with an amplitude that is below the set amplitude (e.g., an amplitude that is not high enough to substantially change the patient's heart rate).

Based on the heart rates HRb, HRd, and HRa, the VNS system 6000 (e.g., a controller of the VNS) may be configured to determine if the stimulation evokes a heart rate change in the patient, for example, above a threshold value and/or if the patient experiences an EMG event (e.g., the EMG sensor records EMG data above a threshold value). This may entail calculating a level of heart rate change based on the heart rate HRd measured during the stimulation and one or both of the heart rates HRb and HRa measured before and after the stimulation is provided, and determining whether the calculated level of heart rate change is greater than a threshold value or falls outside of a set range of heart rate values. In some examples, these determinations may be based only on the heart rates HRd and HRb or only on the heart rates HRd and HRa. Through this iterative process, the stimulation amplitude can be increased until at least one of a lowest amplitude that triggers an EMG event (referred to herein as an EMG amplitude) is determined or an amplitude associated with the neural fulcrum (referred to herein as a NF amplitude) is determined. The NF amplitude will typically be higher than the EMG amplitude. The time period of each iteration (e.g., the time between beginning stimulation of two iterations) of the iterative process may be much shorter than conventional titration processes. For example, the time period may be equal to or less than 24 hours, 12 hours, 6 hours, 4 hours, 2 hours, 1 hour, 30 minutes, 15 minutes, or 5 minutes. Accordingly, the EMG and NF amplitudes can be quickly determined and used to determine an appropriate stimulation amplitude to safely and effectively treat seizures, depression, and/or one or more other medical conditions.

This iterative process may be performed for each electrode on a nerve cuff of the VNS to determine an EMG amplitude and/or a NF amplitude for each of multiple electrodes on the nerve cuff. During the iterative process for a set electrode, the electrode may be activated as a cathode and, in some examples, no other electrodes of the VNS are activated as cathodes. The housing of the VNS may be used as the return anode, or one of the other electrodes on the nerve cuff may be activated as the return anode. Because different electrodes of the nerve cuff are generally differently positioned within the patient and have different impacts on the vagus nerve upon providing stimulation, the EMG and NF amplitudes may each differ from electrode to electrode.

Based on the EMG and/or NF amplitudes, the VNS system may select one or more electrodes to provide stimulation with (e.g., to be activated as cathodes) and/or an amplitude of the stimulation to be provided for each of the selected electrode(s). Selecting the amplitude may entail determining multiple acceptable values (e.g., an acceptable range of amplitudes) and selecting an amplitude from among the multiple acceptable values. For example, the VNS system may select an amplitude for each electrode between the EMG and NF amplitudes determined for said electrode, or within some other range based on the EMG and NF amplitudes for said electrode. The VNS system may continue to monitor (e.g., periodically measure) various biometrics, such as heart rate and EMG, of the patient after selecting the electrodes and associated amplitudes, and may adjust stimulation (e.g., adjust the selected electrodes and/or associated amplitudes) based on the monitored biometrics (e.g., in conjunction with the data obtained during the iterative titration process).

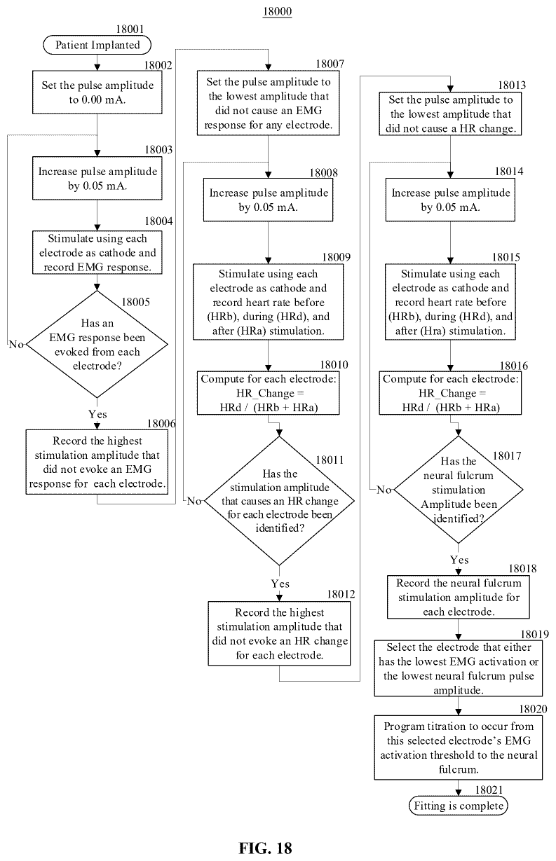

FIG. 18 is flowchart showing a method 18000 for VNS titration according to some examples. The method 18000 may be used with (e.g., performed by) any VNS system described herein, such as the VNS systems 1000 and 6000 of FIGS. 1 and 6A-6C. For example, the operations described with reference to the method 18000 may be performed at least in part by one or more controllers or the system, such as by the implantable controller 6120, the external controller 6220, and/or a controller of the server 6400.

The method 18000 may begin with the VNS being implanted in a patient at a first operation 18001. During a second operation 18002, an initial pulse amplitude of the stimulation provided by the VNS may be set (e.g., via a clinician programmer) at a low level, such as 0 mA. During a third operation 18003, the pulse amplitude may then be increased by a set amount (e.g., by 0.05 mA). At a fourth operation 18004, stimulation may then be provided using each electrode of the VNS as a cathode and with the pulse amplitude set in the third operation 18003, and an electromyogram (EMG) response is evaluated during a fifth operation 18005 to determine if the stimulation provided during the fourth operation 18004 evoked an EMG response from the patient. During the fourth operation 18004, each electrode of the VNS (e.g., of the nerve cuffs 6124) may be activated as a cathode without any of the other electrodes being activated as cathodes. This can allow an evaluation (during the fifth operation 18005) of whether each of the electrodes evokes the EMG response with the stimulation amplitude set at the third operation 18003. In some examples, the housing 1602 may be used as a return (or anode) electrode during the stimulations of the fourth operation 18004. In some other examples, one or more other electrodes of the VNS 6100, besides the activated cathode, may be used as the return (or anode) electrode.

If, during the fifth operation 18005, no EMG response is detected (e.g., by at least one of the cathodes tested during the fourth operation 18004), the method 18000 would return to the third operation 18003 and the pulse amplitude would be increased further. However, if during the fifth operation 18005 a response is detected (e.g., by every cathode tested during the fourth operation 18004), the method 18000 may proceed to a sixth operation 18006. Thus, the third, fourth, and fifth operations 18003, 18004, and 18005 may establish at least part of a logic loop that gradually increases the stimulation amplitude until a stimulation amplitude for each of the tested cathodes has been determined to evoke an EMG response from the patient. These stimulation amplitudes that respectively evoke an EMG response may be referred to as EMG amplitudes. During the sixth operation 18006, for each of the tested electrodes, a stimulation amplitude that did not evoke an EMG response (e.g., a stimulation amplitude just below the EMG amplitude) by the electrode may be recorded.

During a seventh operation 18007, the pulse amplitude may be set to a lowest amplitude that did not cause an EMG response for any of the tested electrodes. For example, this may correspond to a lowest of the amplitudes recorded during the sixth operation 18006 (e.g., an amplitude just below the lowest of the plurality of EMG amplitudes. Next, the pulse amplitude is increased (e.g., by 0.05 mA) during an eighth operation 18008, and stimulation is provided during a ninth operation 18009 with each of the electrodes as a cathode and using the amplitude set during the seventh operation 18007. During the ninth operation 18009, for each of the electrodes, the subject's heart rate is measured before (“HRb”), during (“HRd”), and after (“HRa”) the stimulation with the electrode. A change in heart rate (“HR_Change”) is computed during a tenth operation 18210 for each electrode and using the heart rates HRb, HRd, and HRa measured during the ninth operation 18009 for said electrode. In this example, the change in heart rate HR_Change is calculated as the ratio of the heart rate HRd to the sum of the heart rates HRb and HRa. The heart rate change HR_Change may be calculated in other ways, such as by a ratio of HRd to the average of HRb and HRa, a ratio of HRd to HRb, a ratio of HRd to HRa, a difference between HRd and HRb, or a difference between HRd and HRa. During an eleventh operation 18011, it may be determined for each of the electrodes, and based on the heart rate change HR_Change determined for the electrode during the tenth operation 18010, whether the stimulation provided during the ninth operation 18009 for the electrode caused a change in the patient's heart rate. In some examples, this determination may be made based on whether the heart rate change HR_Change falls outside of a set range, is above an upper threshold value, or falls below a lower threshold value. For example, the determination may be made based on whether the heart rate change HR_Change deviates by more than a set amount (e.g., more than 2%, 3%, 5%, or 10%) from a value of the heart rate change HR_Change when all three of HRb, HRd, and HRa are the same. As a demonstrative example, when HR_Change is calculated as the ratio of HRd to the sum of HRb and HRa, it has a value of ½ when HRd, HRb, and HRa are all the same. It may be determined that the stimulation causes a change in heart rate if the heart rate change HR_Change deviates by more than 5% of this ½ value (i.e., falls outside of the range of 0.475 to 0.525).

After the heart rate changes HR_Change for each of the tested electrodes is determined in the tenth operation 18010, it may be determined during an eleventh operation 18011 whether a stimulation amplitude has been identified that causes a noticeable heart rate change (e.g., that causes HR_Change to satisfy criterion as described herein) for all of the tested electrodes. If so, then the method 18000 may proceed to the twelfth operation 18012. However, if any of the tested electrodes are not determined to cause a noticeable heart rate change, then the method may return to the eighth operation 18008 and the amplitude may be increased again. Thus, the eighth to eleventh operations 18008 to 18011 may form a second logic loop that gradually increases the stimulation amplitude from the level set during the seventh operation 18007 until a stimulation amplitude is determined for each of the tested electrodes that results in a noticeable heart rate change. These stimulation amplitudes that cause the noticeable heart rate change may be referred to as HR amplitudes, may differ from electrode to electrode, and may be recorded for each electrode in response to being determined.

During the twelfth operation 18012, a highest stimulation amplitude that did not evoke a noticeable HR change for each electrode may be recorded. In some examples, this may correspond to an amplitude just below the HR amplitude determined for each electrode during the eleventh operation 18011. Next, during a thirteenth operation 18013, the pulse amplitude is set (e.g., via the clinician programmer) to the lowest amplitude that did not cause a heart rate change. The stimulation amplitude may be increased (e.g., by 0.05 mA) during a fourteenth operation 18014, and stimulation may be provided during a fifteenth operation 18015 for each electrode as a cathode and using the amplitude set during the fourteenth operation 18014. During the fifteenth operation 18015, the heart rates HRb, HRd, and HRa may be determined before, during, and after the stimulation is provided for each electrode for which stimulation is provided. The heart rate change HR_Change may then be determined during a sixteenth operation 18016 for each electrode and based on the HRb, HRd, and HRa values measured while the stimulation was being provided by said electrode during the fifteenth operation 18015. It may then be determined during a seventeenth operation 18017, for each electrode whether the neural fulcrum has been reached for said electrode. If, the stimulation amplitude corresponding to the neural fulcrum has been identified for each of the tested electrodes, then the method 18000 may proceed to the eighteenth operation 18018. These stimulation amplitudes associated with the neural fulcrum may be referred to as NF amplitudes and may be recorded as they are determined. If the NF amplitude has not been identified for all of the tested electrodes, then the method 18000 may return to the fourteenth operation 18014 and the amplitude may be increased again. Thus, the fourteenth to seventeenth operations 18014 to 18017 may form at least part of another logic loop that gradually increases the amplitude until the NF amplitude has been determined for each electrode of the VNS. The NF amplitudes may be recorded during the eighteenth operation 18018.

Stimulation may then be provided based on the data (e.g., the EMG amplitudes and/or the NF amplitudes) collected up through the eighteenth operation 18018. For example, the electrode that has the lowest EMG amplitude and/or that has the lowest NF amplitude may be selected for stimulation during a nineteenth operation 18019, and the VNS may be configured to apply stimulation during a twentieth operation 18020 using the selected electrode and with a pulse amplitude based on the selected electrode's EMG amplitude and NF amplitudes. For example, the VNS may be configured to set the pulse amplitude within a range between the selected electrode's EMG amplitude and NF amplitude. In some examples, the VNS may be configured to titrate a pulse amplitude of the selected electrode between these two thresholds. The method ends at a twenty-first operation 18021.

In some examples, the VNS system may be configured to increase or titrate the pulse amplitude of stimulation, between the selected electrode's EMG amplitude and the NF amplitude, based on an evaluation of the subject's heart rate and/or a change in the subject's heart rate. For example, the present systems may be configured to monitor the subject's heart rate using any of the sensors described herein, and to titrate the pulse amplitude upwards when a positive change in heart rate is detected, with the pulse amplitude associated with neural fulcrum (e.g., the NF amplitude) operating as an upper endpoint. In some examples, the VNS system may be configured to decrease the pulse amplitude when a negative change in heart rate is detected at a pulse amplitude level below the pulse amplitude associated with the neural fulcrum. In some examples, the VNS system is configured to apply a pulse amplitude configured to maintain a subject's heart rate within a predetermined range. For example, the VNS system may be configured to pause stimulation, or decrease the pulse amplitude of stimulation, when the subject's heart rate has increased by 1, 2, 3, 4, 5, 6, 7, 8, 9, or 10 bpm compared to a set (e.g., predetermined) value or a baseline value established prior to the initiation of stimulation. In other examples, stimulation may be paused, or the pulse amplitude of stimulation may be decreased, when the subject's heart rate increases by 1, 2, 3, 4, 5, 6, 7, 8, 9 or 10% compared to a set value or a baseline value established prior to the initiation of stimulation. In some examples, stimulation may be paused, or the pulse amplitude of stimulation may be decreased, when the subject's heart rate increases by an amount or percentage within a range defined by any pair of endpoints selected from either of the foregoing lists).

In some examples, the pulse amplitude of stimulation is adjusted (e.g., at operations 18003, 18008, and/or 18014) by different amounts (e.g., by 0.01, 0.02, 0.03, 0.04, 0.05, 0.06, 0.07, 0.08, 0.09, 0.10, 0.11, 0.12, 0.13, 0.14, 0.15, 0.016, 0.17, 0.18, 0.19, 0.20, 0.21, 0.22, 0.23, 0.24, 0.25, 0.30, 0.35, 0.40, 0.45, or 0.50 mA, or by an amount within a range defined by a pair of endpoints selected from any of the foregoing amounts). The pulse amplitude increase applied at each of the aforementioned steps may be the same or independently selected. In some examples, the increase in pulse amplitude may be constant, whereas in others it may increase at a variable rate (e.g., the rate of increase in pulse amplitude may be progressively lowered, allowing for more precise identification as to the threshold that evokes an EMG response). Similarly, the initial pulse amplitude may be variable in some examples. Here, a starting value of 0.00 mA was selected, but in other cases a non-zero starting level may be applied.

In some examples, stimulus-evoked EMG activity may be evaluated within a window (e.g., 1.0, 1.5, 2.0, 2.5, 3.0, 3.5, 4.0, 4.5, 5.0, 5.5, 60.0, 6.5, 7.0, 7.5, 8.0, 8.5, 9.0, 9.5 10.0, 20.0, 30.0, 40.0, 50.0, 60.0, 70.0, 80.0, 90.0, 100.0, 120.0, 140.0, 160.0, 180.0, 200.0, 220.0, 240.0, 260.0, 280.0, or 300.0 ms, or a window comprising an amount of time within a range defined by any pair of the foregoing lengths of time). In some examples, an EMG response is determined to occur when stimulation of an electrode results in an EMG voltage above a set threshold (e.g., measured in mV). In some examples, an EMG response is determined to occur when stimulation of an electrode results in an EMG voltage exceeding a pre-activation baseline level by a predetermined percentage (e.g., by at least 10, 20, 30, 40, or 50%).

In some examples, the subject's heart rate may be detected using an inertial, electrical, electromagnetic, ultrasound, or optical sensor, which may, for example, be any of the implantable sensors 6111 and/or any of the external sensors 6211. For example, a photoplethysmography (PPG) sensor may be located on a wearable device so that the PPG sensor is in contact with a subject's skin. The PPG sensor may detect blood flow beneath the subject's skin, and this information may be used to determine the subject's heart rate. In another example, the heart rate sensor may be an electromagnetic sensor (e.g., located on a chest strap). In some examples, an IMU, a phonocardiography (PCG) sensor, or any other sensor capable of detecting a signal that is directly or indirectly indicative of a subject's heart rate. The subject's heart rate may be determined using a single obtained using a single sensor or a plurality of sensors.

In the example shown in FIG. 18, the system may be configured to apply stimulation at a pulse amplitude between the selected electrode's EMG amplitude and the NF amplitude. In some other examples, the electrode's EMG amplitude and/or NF amplitude for one or more electrodes may be used to set or control treatment parameters without functioning as endpoints. For example, an alternative system may be configured to apply stimulation with an electrode using a pulse amplitude above that of the electrode's NF amplitude. In that case, sensor data may be collected to monitor the subject's heart rate (e.g., to ensure that stimulation does not cause the subject's heart rate to decrease below a predetermined safety threshold). In some examples, the system may be configured to decrease the pulse amplitude when a negative change in heart rate is detected at a pulse amplitude level above the NF amplitude. In some examples, the system is configured to pause stimulation (or to decrease the pulse amplitude of stimulation) when the subject's heart rate has decreased by 1, 2, 3, 4, 5, 6, 7, 8, 9, or 10 bpm compared to a set value or a baseline value established prior to the initiation of stimulation. In other examples, stimulation may be paused, or the pulse amplitude of stimulation may be decreased, when the subject's heart rate decreases by 1, 2, 3, 4, 5, 6, 7, 8, 9 or 10% compared to a set value or a baseline value established prior to the initiation of stimulation. In some examples, stimulation may be paused, or the pulse amplitude of stimulation may be decreased, when the subject's heart rate decreases by an amount or percentage within a range defined by any pair of endpoints selected from either of the foregoing lists).

In some examples, the system may be configured to apply stimulation with an electrode at a pulse amplitude within 0.1, 0.2, 0.3, 0.4, 0.5, 0.6, 0.7, 0.8, 0.9, or 1.0 mA of the NF amplitude of the electrode. In some examples, the system may alternatively be configured to apply stimulation at a pulse amplitude within a range that deviates from the electrode's NF amplitude by up to 10, 20, 30, 40, or 50%. For example, in a situation where the neural fulcrum is associated with a pulse amplitude of 2 mA; a 50% deviation in this hypothetical case would encompass a range of 0.5 to 1.5 mA.

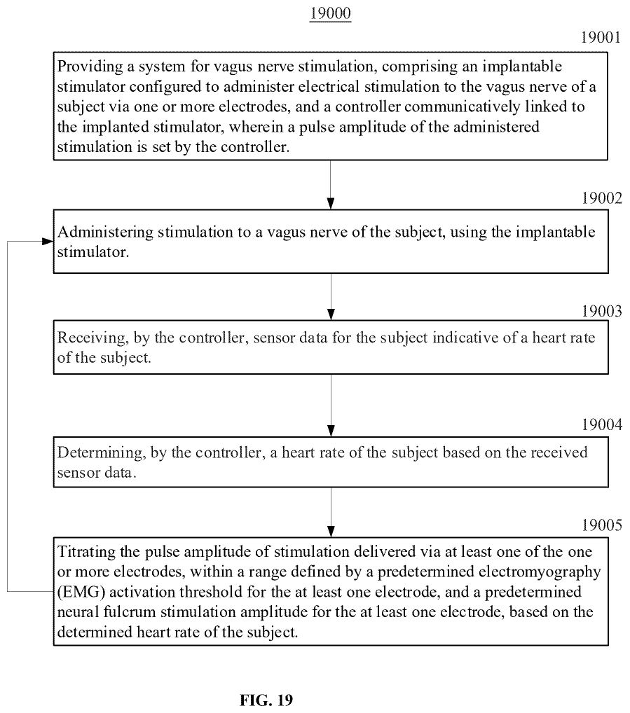

FIG. 19 is a flowchart of a method according to some examples for treating a subject using any of the VNS systems described herein. In this non-limiting example, treatment begins at a first operation 19001 with the subject being provided with a system for VNS, comprising a VNS (e.g., an implantable stimulator) configured to administer electrical stimulation to the vagus nerve of the subject via one or more electrodes, and a controller communicatively linked to the VNS, wherein a pulse amplitude of the administered stimulation is set by the controller. Next, the vagus nerve of the subject is stimulated using the VNS during a second operation 19002. The controller may receive sensor data indicative of a heart rate of the subject during a third operation 19003 and use this sensor data to determine the subject's heart rate during a fourth operation 19004. During a fifth operation 19005, the pulse amplitude of stimulation delivered via at least one of the one or more electrodes may then be titrated, by the controller, within a range defined by a predetermined EMG activation threshold for the at least one electrode, and a predetermined neural fulcrum stimulation amplitude for the at least one electrode, based on the determined heart rate of the subject. In other examples, additional sensor data (e.g., movement data collected using an IMU) may also be collected and used by the controller as a parameter for determining the level of stimulation to apply during this titration process.

In some examples, the VNS systems and methods described herein may be used to treat epilepsy (e.g., epileptic seizure frequency) as an exemplary treatment, but may be employed to treat other medical conditions such as depression, inflammation (e.g., related to maladies such as rheumatoid arthritis), brain diseases (e.g., Alzheimer's disease, dementia, traumatic brain injury, Parkinson's disease, and ischemic stroke), heart conditions (e.g., heart failure, cardiovascular disease), and for improving heart function, pain-related conditions (e.g., Migraines, cluster headaches, and pain-related disorders), inflammatory conditions (e.g., inflammatory bowel disease, rheumatoid arthritis, and autoimmune diseases, psychiatric conditions (e.g., anxiety disorders, and PTSD), and other conditions (e.g., diabetes, obesity, and sleep disorders). An example method of treating any such diseases or conditions may comprise fitting a subject with a system according to the present disclosure and/or using any of the present systems to reduce one or more symptoms of the foregoing diseases and conditions.

### VII. Patient Fall Detection

As discussed herein, VNS systems can be used to treat epilepsy by delivering stimulation pulses to a vagus nerve of a patient to prevent or minimize the duration and severity of an epileptic seizure. During a seizure, a patient's heart rate can abruptly rise (ictal tachycardia) in a relatively predictable manner, and the seizure can be detected with a degree of accuracy by monitoring the patient's heart rate. However, the patient's heart rate can also rise due to other medical conditions and/or the patient's physical activities (e.g., sudden increases in exertion), and detecting a seizure based solely on changes in heart rate can sometimes lead to incorrect determinations of whether the patient is or is not experiencing a seizure or the onset of a seizure. Accordingly, there is a need to improve systems and processes for detecting seizures to improve epilepsy treatment.

Any of the VNS systems described herein, such as the VNS systems 1000 and 6000 of FIGS. 1 and 6A-6C, may be configured to detect whether a patient has fallen and to determine whether the patient is experiencing a seizure or the onset of a seizure based on both the fall detection and the patient's heart rate. A patient experiencing a seizure will usually also fall to the ground due to a loss of control caused by the seizure. Thus, the accuracy of detecting a seizure can be improved if it is based on both detection of a rise in heart rate and detection of a fall. On the other hand, if a fall is detected but not a rise in heart rate, it may be determined that the patient fell for a reason other than because the patient experienced a seizure, and VNS stimulation may not be provided. Also, if a rise in heart rate is detected but not a fall, it may be determined that the patient's heart rate rose for a reason other than because the patient experienced a seizure. In this case, the VNS system may or may not provide VNS stimulation. In some cases, VNS stimulation may still be provided, for example, to account for the possibility that the patient is experiencing a seizure while maintaining an upright position or if it is determined that the patient was in a horizontal position prior to experiencing the rise in heart rate (in which case the seizure would not trigger a fall).

One or more of the sensors of the VNS system (e.g., one or more of the implantable sensors 6111 and/or one or more of the external sensors 6211) may be configured to detect a fall and/or the patient's bodily position (e.g., whether the patient is in an upright position or a horizontal position). For example, one or more of the implantable sensors 6111 and/or one or more of the external sensors 6211 may include an inertial measurement unit (IMU), an accelerometer (XL), and/or a gyroscope configured to detect at least one of a fall, the patient's bodily position, and/or the patient's heart rate. For example, all three functions may be achieved via a single sensor (e.g., an IMU), which may be implantable in the patient (e.g., may be the second implantable sensor 6111B forming part of the VNS 6100) or an external sensor. In some other examples, an IMU sensor (e.g., an implantable IMU sensor) may be used to detect a fall and/or bodily position, and a separate sensor (e.g., an external sensor, such as a sensor on a wearable device) may be used to measure the patient's heart rate.

The VNS system (e.g., a controller of the VNS system, such as the implantable controller 6120, the external controller 6220, and/or a controller of the server 6400) may be configured to collect the sensed data (e.g., fall detection data, bodily position data, and/or heart rate data) from the one or more sensors and to detect a seizure, or the onset of a seizure, based on the sensed data as described above. In response to detecting the seizure, the VNS system may provide (turn on or activate stimulation), or adjust (e.g., increase the intensity or duration of stimulation by increasing stimulus pulse amplitude, stimulus pulse width, stimulus pulse frequency or duty cycle), VNS stimulation in any manner described herein.

Based on the sensed data, the VNS system may also be configured to transmit an alert signal to an external device, such as the patient remote 1300A, the clinician programmer 1300B, the electronic device 6200, or to another device that may, for example, be in the possession of a caregiver, family member, or emergency service. The alert signal may signal that the patient has fallen and/or is experiencing a seizure. In some examples, the VNS system may be configured to transmit the alert even if it detects a fall without a seizure, and the alert signal in some such examples may indicate that the patient has fallen and needs assistance. In some cases, the alert signal is transmitted (e.g., via a cellular or Wi Fi connection) to a third party device, such as a device at a medical facility (e.g., a hospital) or other third party entity that may respond to the alert signal that the patient needs assistance (e.g., with a fall or with a seizure). The alert signal may be sent via any communication form, such as a cellular, Wi Fi, RF, or Bluetooth connection, or via a text message, telephone call, or other communications method. The communication form chosen may depend on which device the alert signal is being sent to. For example, a Bluetooth connection may be chosen for transmitting the alert signal to nearby external devices, such as to the patient remote 1300A or electronic device 6200.

In some examples, the VNS 6100 (or one of the first implantable sensors 6111A) may initialize an IMU and/or XL when it is powering up for the first time or from a low-power mode. The IMU and/or XL may be set up to detect free fall during this initialization process. The initializing process may include commanding free fall detection through built-in commands, registers and dedicated hardware, or it could include programming the IMU and/or XL to detect Og acceleration on 3 axes (e.g., 3 mutually orthogonal axes), which is an indicator of true free fall, and to issue an interrupt if this condition is true for a set period of time. This would then issue an interrupt to the controller when a free fall occurs, and the controller could then determine that the fall event has occurred.

The signals from the accelerometer(s) on the IMU and/or the XL may be processed to determine the heart rate of the patient in some examples (e.g., in conjunction with being processed to detect the fall event). The heart rate may then be monitored to detect ictal tachycardia. In some examples, it may be beneficial to obtain a heart rate from an external device such as a smartwatch, and this information may then be transmitted to a controller, such as to the controller of the Patient Remote and/or of the VNS. The Patient Remote or IPG could then monitor the heart rate for ictal tachycardia.

If the patient-fallen event took place without ictal tachycardia being detected, stimulation to the vagus nerve may or may not be desired. A clinician may program the IPG whether to deliver vagus nerve stimulation in this case. If vagus nerve stimulation is not desired in this case, the IPG may still command the Patient Remote via an RF communications channel to notify caregivers and/or emergency services of the patient's fall via a text message, telephone call, or another communications method.

FIG. 20 is a flowchart diagram of a method 20000 for detecting a fall event, and using the detected fall event, in the operations of a VNS system according to some examples. The method may be performed by any of the VNS systems described herein, such as any one of the VNS systems 1000 and 6000 described with reference to FIGS. 1 and 6A-6C.

The method 20000 may include a first operation 20002 of measuring, via one or more sensors, heart rate data and at least one of movement data or bodily position data. The one or more sensors may be any combination of the sensors of the VNS system, such as any one or more of the implantable sensors 6111 and/or any one or more of the external sensors 6211. In some examples, a single sensor (e.g., an implantable IMU, implantable accelerometer, or an implantable gyroscope) may be used to collect all three of the heart rate data, the movement data, and the bodily position data. In some other examples, two or more sensors may be used. For example, a first sensor (e.g., an implantable IMU, implantable accelerometer, or an implantable gyroscope) may be used to collect one or both of the movement data and the bodily position data, and a second sensor (e.g., an external sensor) may be used to collect the heart rate data. The sensors may transmit their sensed data to one or more controllers of the VNS system (e.g., the implantable controller 6120, the external controller 6220, and/or a controller of the server 6400) for processing.

During a second operation 20004, a controller may detect for ictal tachycardia based on the measured heart rate data. During a third operation 20006, the controller (or, in some other examples, a different controller) may detect for a fall event (i.e., whether the patient has fallen) based on at least one of the movement data or the bodily position data.

The method 20000 may include a fourth operation 20008 of determining, by the controller (or, in some other examples, a different controller) whether a seizure has occurred based on whether the ictal tachycardia is detected and whether the fall event is detected. In some other examples, the controller may make this determination directly based on the heart rate data and one or both of the movement data or the bodily position data (e.g., without first detecting for ictal tachycardia and a fall event). Determining that a seizure has occurred based on this data may be performed in any manner described herein.

In response to determining that the seizure has occurred, a VNS (e.g., the VNS 6100) may begin or increase stimulation (e.g., increase pulse amplitude of pulse stimulation) during a fifth operation 2010. During a sixth operation 20012, the controller may transmit an alert signal (e.g., to an external device) in response to detecting that a fall event and/or a seizure has occurred. The alert signal may indicate that the patient needs assistance due to a fall and/or seizure, as described herein.

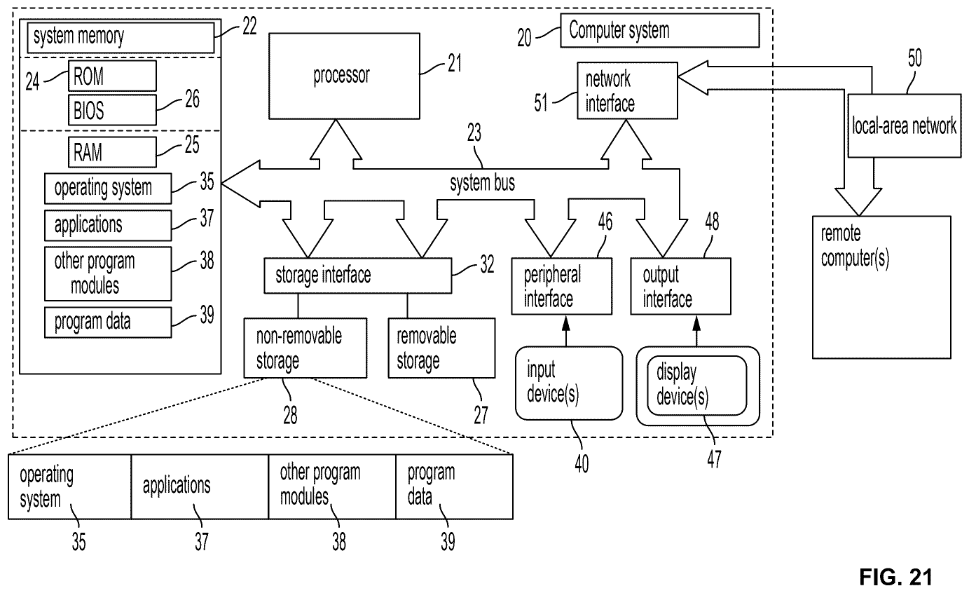

FIG. 21 is a block diagram of various components of a computer system 20 according to some examples that may be used as one or more of the computing devices of the VNS systems described herein. For example, the computer system 20 includes features that may be used in (e.g., used as) the patient remote 1300A, the clinician programmer 1300B, the electronic device 6200, the remote server 6400, or the cloud-based infrastructure 6410.

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. The computer system 20 may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a wearable electronic device, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may include a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable codes implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may include a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can include a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

In closing, it is to be understood that although examples of the present specification have been described herein, one skilled in the art will readily appreciate that these disclosed examples are only illustrative of the principles of the subject matter disclosed herein. Therefore, it should be understood that the disclosed subject matter is in no way limited to the specific examples disclosed herein, unless expressly stated as such. In addition, those of ordinary skill in the art will recognize that certain changes, modifications, permutations, alterations, additions, subtractions, and sub-combinations of the disclosed examples can be made in accordance with the teachings herein without departing from the spirit of the present disclosure. It is therefore intended that the following appended claims and claims hereafter introduced are interpreted to include all such changes, modifications, permutations, alterations, additions, subtractions, and sub-combinations as are within their true spirit and scope.

Certain examples of the present disclosure are described herein, including the best mode known to the inventors for carrying out the invention. Of course, variations on these described examples will become apparent to those of ordinary skill in the art upon reading the foregoing description. The inventors expect skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described examples in all possible variations thereof is encompassed by the invention unless otherwise indicated herein or otherwise clearly contradicted by context.

Groupings of different examples, elements, or processes of the present disclosure are not to be construed as limitations. Each group member may be referred to and claimed individually or in any combination with other group members disclosed herein. Itis anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

What is claimed is:

1. A stimulation system, comprising:
an implantable medical device (IMD) configured to provide electrical stimulation to tissue;
a controller configured to control the electrical stimulation provided by the IMD;
an implantable sensor configured to measure first heart rate data and to transmit the first heart rate data to the controller;
an external sensor configured to measure second heart rate data and to transmit the second heart rate data to the controller; and
an external electronic device communicatively coupled to the IMD,
wherein the controller is configured to selectively operate the IMD in an internal sensor mode, whereby the controller controls the electrical stimulation provided by the IMD based on the first heart rate data, or an external sensor mode, whereby the controller controls the electrical stimulation provided by the IMD based on the second heart rate data.

2. The stimulation system of claim 1, wherein the controller is configured to not operate, or to not communicate with, the implantable sensor when operating the IMD in the external sensor mode.

3. The stimulation system of claim 1, wherein the controller is configured to operate the IMD in the internal sensor mode in response to determining that a threshold criterion is satisfied.

4. The stimulation system of claim 3, wherein the threshold criterion comprises the controller being communicatively disconnected from the external sensor for a threshold time period and/or a distance between the external sensor and an implantable component of the stimulation system being greater than a threshold distance.

5. The stimulation system of claim 3, wherein the threshold criterion comprises at least one of the following:
a signal quality of the second heart rate data being below a threshold value;
a scheduled day and/or intraday time period;
the implantable sensor being ranked higher than the external sensor;
a battery level of the external sensor being below a threshold level; or
a processing capability value of the external sensor being below a threshold value.

6. The stimulation system of claim 3, wherein the electronic device is configured to receive user input via a user interface, and the threshold criterion comprises receiving a user control signal from the electronic device.

7. The stimulation system of claim 1, wherein the controller is configured to operate the IMD in the external sensor mode in response to determining that a threshold criterion is satisfied.

8. The stimulation system of claim 7, wherein the threshold criterion comprises the controller being communicatively connected to the external sensor for a threshold time period and/or a distance between the external sensor and an implantable component of the stimulation system being less than a threshold distance.

9. The stimulation system of claim 7, wherein the threshold criterion comprises at least one of the following:
a signal quality of the second heart rate data being above a threshold value;
a scheduled day and/or intraday time period;
the external sensor being ranked higher than the implantable sensor;
a battery level of the external sensor being above a threshold level; or
a processing capability value of the external sensor being above a threshold value.

10. The stimulation system of claim 1, wherein the controller is further configured to selectively operate the IMD in a multi-sensor mode, whereby the controller controls the electrical stimulation provided by the IMD based on both the first heart rate data and the second heart rate data.

11. The stimulation system of claim 1, wherein the controller is part of the IMD.

12. The stimulation system of claim 1, wherein the controller is part of the electronic device.

13. The stimulation system of claim 1, wherein the external sensor is part of the electronic device.

14. The stimulation system of claim 1, wherein the external sensor is separate from the electronic device.

15. The stimulation system of claim 1, wherein the implantable sensor is part of the IMD.

16. The stimulation system of claim 1, wherein the implantable sensor is separate from the IMD.

17. The stimulation system of claim 1, wherein the implantable sensor comprises at least one of an inertial measurement unit (IMU) or an accelerometer.

18. The stimulation system of claim 1, wherein the electronic device comprises a wearable device selected from among a watch, a ring, a bracelet, a band, a necklace, or an earring.

19. The stimulation system of claim 1, wherein the electronic device comprises a stationary device, configured to be operated while positioned on a surface, or a portable device, configured to be operated while being held or carried.

20. The stimulation system of claim 1, wherein the IMD comprises a cuff electrode configured to stimulate a vagus nerve, the cuff electrode comprising a plurality of electrode contacts configured to circumferentially surround the vagus nerve.

21. The stimulation system of claim 20, wherein the controller is configured to independently activate each of the plurality of electrode contacts as a cathode or as an anode.

22. The stimulation system of claim 20, wherein the IMD comprises a conductive housing containing at least some components of the IMD and being exposed to an outside of the IMD, and
wherein the controller is configured to selectively activate the conductive housing as an anode.

23. The stimulation system of claim 1, wherein the IMD comprises a receiver coil, and
wherein the stimulation system comprises a wireless power transfer device, comprising:
a first coil oriented along a first axis;
a second coil oriented along a second axis different from the first axis and positioned above the first coil along a direction perpendicular to the first and second axes; and
a driver configured to differentially drive the first and second coils to generate a magnetic field and to control a direction of the magnetic field at the receiver coil.

24. The stimulation system of claim 23, wherein the wireless power transfer device comprises:
a transmission component housing the first and second coils;
an electronics component housing the driver; and
a cable physically and electrically connecting the electronics component to the transmission component.

25. The stimulation system of claim 24, comprising a support garment for the wireless power transfer device, the support garment comprising:
a first chest part configured to cover a first sagittal side of a wearer's chest;
a second chest part configured to cover a second sagittal side of the wearer's chest; and
a neck part coupled between the first and second chest parts and configured to cover a back of the wearer's neck.

26. The stimulation system of claim 25, wherein the support garment comprises at least one of:
a first fastener on the first chest part and configured to attach to the transmission component;
a second fastener on the second chest part and configured to attach to the electronics component; or
a cable holder on the neck part and configured to secure the cable along the neck part.

27. A stimulation system, comprising:
an implantable medical device (IMD) comprising an implantable pulse generator (IPG) configured to generate a stimulation current, a stimulation lead coupled to the IPG, and a stimulation electrode on the stimulation lead and configured to receive the stimulation current from the IPG through the stimulation lead;
a controller;
an implantable sensor configured to measure first biometric data and to transmit the first biometric data to the controller;
an external sensor configured to measure second biometric data and to transmit the second biometric data to the controller; and
an external electronic device communicatively coupled to the implantable stimulator and configured to receive input data via a user interface,
wherein the controller is configured to control the stimulation current generated by the IPG based selectively on the first biometric data or the second biometric data.

28. The stimulation system of claim 27, wherein the controller is further configured to selectively control the stimulation current generated by the IPG based on the first biometric data, based on the second biometric data, or based on both the first and second biometric data.

29. The stimulation system of claim 27, wherein the controller is configured to determine a heart rate based on at least one of the first biometric data or the second biometric data.

30. The stimulation system of claim 29, wherein the controller is configured to perform a titration process comprising:
determining a normal heart rate value based on at least one of the first biometric data or the second biometric data;
performing an iterative neural fulcrum identification (NFI) operation, comprising:
(a) generating the stimulation current having a set amplitude,
(b) determining a transient heart rate value based on at least one of the first biometric data or the second biometric data measured while providing the stimulation current of process (a), and
(c) determining a heart rate change (HRC) value based on the normal heart rate value and the transient heart rate value of process (b);
performing the NFI operation one or more additional times, each time at a higher set amplitude than the previous time; and
determining, based on the plurality of HRC values determined during the NFI operations, a neural fulcrum amplitude associated with a neural fulcrum response.

31. The stimulation system of claim 30, wherein the controller is configured to generate the stimulation current with an amplitude based on the neural fulcrum amplitude.

32. The stimulation system of claim 30, wherein the NFI operation comprises a process (n), before process (a), of determining a normal heart rate value while the stimulation current is not provided or is provided with an amplitude less than the set amplitude of process (a), and the heart rate change value of process (c) is determined using the normal heart rate value determined during process (n).

33. The stimulation system of claim 30, wherein an inter-NFI time period between processes (a) of two adjacently performed NFI operations is less than 4 hours.

34. The stimulation system of claim 33, wherein the inter-NFI time period is less than 30 minutes.

35. The stimulation system of claim 30, wherein the NFI operation comprises a process (d) of detecting for an electromyography (EMG) response while the stimulation current is provided during process (a),
wherein the titration process comprises determining a lowest EMG amplitude that triggers an EMG response, and
wherein the controller is configured to generate the stimulation current with an amplitude based on both the neural fulcrum amplitude and the lowest EMG amplitude.

36. The stimulation system of claim 29, wherein the controller is configured to detect a seizure, or the onset of a seizure, based on a comparison of the determined heart rate and a personalized ictal tachycardia model.

37. The stimulation system of claim 36, further comprising a memory coupled to the controller and storing the personalized ictal tachycardia model.

38. The stimulation system of claim 36, wherein the personalized ictal tachycardia model is based on a plurality of sets of seizure data, each of the sets of seizure data comprising heart rate data of a single subject while having a seizure.

39. The stimulation system of claim 36, wherein the controller is configured:
to record, for each of a plurality of seizures, a corresponding set of seizure data comprising heart rate data determined based on at least one of the first biometric data or the second biometric data; and
to generate the personalized ictal tachycardia model based on the plurality of sets of seizure data.

40. The stimulation system of claim 36, wherein the controller is configured:
to determine a respiration rate based on at least one of the first biometric data or the second biometric data, and
to detect the seizure, or the onset of the seizure, based further on the determined respiration rate and a personalized ictal apnea respiration response model.

41. The stimulation system of claim 36, wherein the personalized ictal tachycardia model comprises a model parameter, and
wherein the detecting the seizure, or the onset of the seizure, comprises calculating a heart rate parameter, based on at least one of the first biometric data or the second biometric data, and comparing the heart rate parameter to the model parameter.

42. The stimulation system of claim 36, wherein the ictal tachycardia model comprises a discriminative neural network configured to detect the seizure, or the onset of the seizure, based on heart rate data determined based on at least one of the first biometric data or the second biometric data.

43. The stimulation system of claim 27, wherein the implantable sensor or the external sensor comprises at least one of an inertial measurement unit (IMU) or an accelerometer configured to measure movement data,
wherein the controller is configured to detect a fall event based on the movement data, and to detect ictal tachycardia based on at least one of the first biometric data or the second biometric data.

44. The stimulation system of claim 43, wherein the controller is configured to detect the ictal tachycardia based on the movement data.

45. The stimulation system of claim 43, wherein the controller is configured to cause the IMD to begin generating the stimulation current, or to increase a parameter of the stimulation current, in response to detecting both the fall event and the ictal tachycardia.

46. The stimulation system of claim 45, wherein the controller is configured to not cause the IMD to begin generating the stimulation current, or to not increase the parameter of the stimulation current, in response to detecting the fall event without detecting the ictal tachycardia.

47. The stimulation system of claim 42, comprising a transmitter,
wherein the controller is configured to transmit, via the transmitter, an alert signal in response to detecting the fall event.

48. The stimulation system of claim 27, wherein the system is configured to recalibrate the implantable sensor based on the second biometric data and/or to recalibrate the external sensor based on the first biometric data.

49. The stimulation system of claim 48, wherein the system is configured to compare the first biometric data to the second biometric data to determine that one of the implantable sensor or the external sensor is uncalibrated.

50. The stimulation system of claim 27, wherein the controller is an implantable controller that is part of the implantable stimulator,
wherein the electronic device comprises an external controller configured to receive the first and second biometric data, and
wherein the stimulation system is configured to selectively operate:
in a first mode, whereby the implantable controller determines, based on at least one of the first biometric data or the second biometric data, a stimulation parameter for the stimulation current, or
in a second mode, whereby the external controller determines, based on at least one of the first biometric data or the second biometric data, the stimulation parameter for the stimulation current.

51. The stimulation system of claim 50, wherein the stimulation system is configured to transition from the first mode to the second mode in response to a communication link between the implantable controller and the external controller being established, and to transition from the second mode to the first mode in response to implantable controller being communicatively disconnected from the external controller for a threshold time period.

52. A method for providing electrical stimulation to tissue of a subject via an implantable medical device (IMD) implanted within the subject, the method comprising:
controlling, via a controller and during a first time period, a parameter of the electrical stimulation based on first heart rate data measured by an implantable sensor implanted within the subject; and
controlling, via the controller and during a second time period, the parameter of the electrical stimulation based on second heart rate data measured by an external sensor external to the subject.

53. The method of claim 52, wherein, during the second time period, the implantable sensor is not operated or is not communicatively coupled to the controller.

54. The method of claim 52, wherein, during the first time period, the external sensor is communicatively disconnected from the controller.

55. The method of claim 52, comprising, controlling, via the controller and during a third time period, the parameter of the electrical stimulation based on both the first heart rate data and the second heart rate data.
